(19) United States
(12) Patent Application Publication
(10) Pub. No.: US 2021/0115406 A1
(43) Pub. Date: Apr. 22, 2021

(54) HOST-BIOME INTERACTIONS

(71) Applicant: EMULATE, INC., Boston, MA (US)

(72) Inventors: S. Jordan Kerns, Reading, MA (US); Catherine Karalis, Brookline, MA (US); Janna Nawroth, Boston, MA (US); Remi Villenave, Boston, MA (US); Jenifer Obrigewitch, Cambridge, MA (US); Doris Roth, Boston, MA (US); Michael Salmon, Boston, MA (US); Athanasia Apostolou, Brookline, MA (US); David Conegliano, Boston, MA (US)

(21) Appl. No.: 17/088,828

(22) Filed: Nov. 4, 2020

Related U.S. Application Data
(63) Continuation of application No. PCT/US2019/031596, filed on May 9, 2019.
(60) Provisional application No. 62/680,274, filed on Jun. 4, 2018.
(60) Provisional application No. 62/669,156, filed on May 9, 2018.

(51) Int. Cl. C12N 5/071; C12M 3/06; C12M 1/42; C12N 1/20; C12Q 1/04

(52) U.S. Cl. CPC C12N 5/0679; C12M 23/16; C12M 35/08; C12M 35/04; C12N 1/20; C12N 2502/70; C12N 5/0688; C12N 2500/02; C12N 2537/00; C12N 2506/23; C12Q 1/04

(57) ABSTRACT

The present invention relates to a combination of microbes, cell culture systems and microfluidic fluidic systems for use in providing a human Intestine On-Chip with optimal intestinal motility. More specifically, in some embodiments, a microfluidic chip containing intestinal epithelial cells co-cultured with intestinal endothelial cells in the presence of bacteria, such as probiotic bacteria, may find use in providing an Intestine-On-Chip for testing intestinal motility function. In some embodiments, an Intestine On-Chip may be used for identifying (testing) therapeutic compounds continuing probiotic microbes or compounds for inducing intestinal motility for use in treating gastrointestinal disorders or diseases related to intestinal function.

### CROSS-REFERENCE TO RELATED APPLICATIONS

This application is a Continuation of, and claims priority to, co-pending PCT Patent Application Serial No. PCT/US2019/031596, filed May 9, 2019, which claims priority to Provisional Application Ser. Nos. 62/680,274 filed on Jun. 4, 2018 and 62/669,156 filed on May 9, 2018, both now expired, the contents of which are incorporated herein in their entirety.

### FIELD OF THE DISCLOSURE

The present invention relates to a combination of microbes, cell culture systems and microfluidic fluidic systems for use in providing a human Intestine On-Chip with optimal intestinal motility. More specifically, in some embodiments, a microfluidic chip containing intestinal epithelial cells co-cultured with intestinal endothelial cells in the presence of bacteria, such as probiotic bacteria, may find use in providing an Intestine-On-Chip for testing intestinal motility function. In some embodiments, an Intestine On-Chip may be used for identifying (testing) therapeutic compounds continuing probiotic microbes or compounds for inducing intestinal motility for use in treating gastrointestinal disorders or diseases related to intestinal function.

### BACKGROUND

Healthy intestinal function is often disrupted by human conditions including disorders and diseases involving types of bacteria found in the intestines.

There is a need for a better platform to test therapeutic compounds related to bacteria associated with intestinal function.

### SUMMARY OF THE INVENTION

The present invention relates to a combination of microbes, cell culture systems and microfluidic fluidic systems for use in providing a human Intestine On-Chip with optimal intestinal motility. More specifically, in some embodiments, a microfluidic chip containing intestinal epithelial cells co-cultured with intestinal endothelial cells in the presence of bacteria, such as probiotic bacteria, may find use in providing an Intestine-On-Chip for testing intestinal motility function. In some embodiments, an Intestine On-Chip may be used for identifying (testing) therapeutic compounds continuing probiotic microbes or compounds for inducing intestinal motility for use in treating gastrointestinal disorders or diseases related to intestinal function.

In one embodiment, the present invention contemplates a method for modulating a response to an agent, comprising: a) providing a fluidic device comprising first living cells; b) inoculating said device with one or more microorganisms; and c) stimulating said first living cells with a first agent. In one embodiment, said living cells are epithelial cells. In one embodiment, said living cells are intestinal cells. In one embodiment, said living cells are lung cells. In one embodiment, said one or more microorganisms comprise a bacterium, virus, fungus and/or parasite. In one embodiment, said inoculation in step (b) involves using a specific titer or MOI of bacteria. In one embodiment, said first agent is a pro-inflammatory agent. In one embodiment, said pro-inflammatory agent comprises LPS. In one embodiment, said pro-inflammatory agent comprises TNF-α. In one embodiment, said agent comprises a cytokine, immune-modulator. In one embodiment, said agent comprises a second microorganism. In one embodiment, said agent comprises a drug. In one embodiment, the method further comprises measuring drug transport (e.g. across a membrane). In one embodiment, the method further comprises measuring drug efficacy. In one embodiment, the method further comprises measuring drug safety. In one embodiment, the method further comprises measuring cytokine expression. In one embodiment, the method further comprises measuring viability or proliferation of at least some of said microorganisms. In one embodiment, the method further comprising measuring the viability and/or function of said living cells.

In one embodiment, the present invention contemplates a method for measuring the efficacy of an anti-microorganism agent, comprising: a) providing a fluidic device comprising first living cells; b) inoculating said device with a first microorganism; c) culturing said device in the absence of said anti-microorganism agent; d) culturing said device in the presence of said anti-microorganism agent; and e) measuring the viability or function of said first microorganism. In one embodiment, the living cells comprise intestinal cells. In one embodiment, the living cells comprise lung cells. In one embodiment, the first microorganism is a bacterium, virus, fungus, or parasite. In one embodiment, the anti-microorganism agent is an antibiotic, antiviral, antifungal, anti-parasitic agent. In one embodiment, the culturing in the absence of said anti-microorganism agent in step (c) is at least 12 hours, more preferably 24 hours (and most preferably not longer than one week). In one embodiment, the measuring in step (e) comprises quantification methods (described herein).

In one embodiment, the present invention provides a method of culturing a microorganism, comprising: a) providing a fluidic device comprising first living cells; b) inoculating said device with one or more microorganisms capable of developing biofilms; and c) culturing said first living cells and said microorganisms under conditions such that a biofilm is created by said microorganisms. In one embodiment, said first living cells comprise intestinal cells. In one embodiment, said microorganisms comprise bacterial cells. In one embodiment, said device is a microfluidic device. In one embodiment, said first living cells are in a cell layer. In one embodiment, bacterial cells adhere to said first living cells. In one embodiment, microfluidic device comprises first and second microchannels separated by a membrane, said membrane comprising first and second sides, wherein said first side serves as a surface for said first microchannel and said second side serves as a surface for said second microchannel. In one embodiment, culturing comprises introducing fluid at a flow rate. In one embodiment, bacterial cells are selected from the group consisting of nonpathogenic bacterial strains and pathogenic bacterial strains. In one embodiment, the method further comprises d) measuring viability or proliferation of at least some of said microorganisms. In one embodiment, the method further comprises d) measuring the viability and/or function of said living cells. In one embodiment, the method further comprises introducing a drug into said device such that it contacts said biofilm.

In one embodiment, the present invention provides a method of identifying a probiotic bacteria strain, comprising: a) providing: i) a microfluidic device; ii) a plurality of intestinal cell; and iii) a plurality of bacterial cells from a candidate bacteria strain; b) seeding said intestinal cells in said microfluidic device so as to provide an intestinal cell layer having a barrier function; c) contacting said intestinal cells in said intestinal cell layer with said plurality of bacterial cells so as to allow at least some of said bacterial cells to adhere to said intestinal cells; and d) detecting a change in said contacted intestinal cells, thereby identifying said bacterial cells as probiotic. In one embodiment, said microfluidic device comprises first and second microchannels separated by a membrane, said membrane comprising first and second sides, wherein said first side serves as a surface for said first microchannel and said second side serves as a surface for said second microchannel. In one embodiment, the method further comprises introducing fluid at a flow rate prior to, during or after step c). In one embodiment, said change detected in step d) comprises an increase in said barrier function of said contacted intestinal cells. In one embodiment, said increase in barrier function is measured by an increase in the expression of one or more tight junction proteins. In one embodiment, said intestinal cells are primary epithelial cells. In one embodiment, said intestinal cells are patient derived cells. In one embodiment, the method further comprises the step e) adding said probiotic bacteria to a food product.

In one embodiment, the present invention provides a method of identifying a pathogenic bacteria strain, comprising: a) providing: i) a microfluidic device; ii) a plurality of intestinal cell; and iii) a plurality of bacterial cells from a pathogenic bacteria strain; b) seeding said intestinal cells in said microfluidic device so as to provide an intestinal cell layer having a barrier function; c) contacting said intestinal cells in said intestinal cell layer with said plurality of bacterial cells so as to allow at least some of said bacterial cells to adhere to said intestinal cells; and d) detecting a change in said contacted intestinal cells, thereby identifying said bacterial cells as pathogenic. In one embodiment, said microfluidic device comprises first and second microchannels separated by a membrane, said membrane comprising first and second sides, wherein said first side serves as a surface for said first microchannel and said second side serves as a surface for said second microchannel. In one embodiment, said the method further comprises introducing fluid at a flow rate prior to, during or after step c). In one embodiment, said change detected in step d) comprises a decrease in said barrier function of said contacted intestinal cells. In one embodiment, said decrease in barrier function is measured by a decrease in the expression of one or more tight junction proteins. In one embodiment, said intestinal cells are primary epithelial cells. In one embodiment, said intestinal cells are patient derived cells. In one embodiment, said bacteria form a biofilm after step c).

In one embodiment, the present invention provides a method of culturing anaerobic bacteria, comprising: a) providing: i) a microfluidic device; ii) a plurality of intestinal cell; and iii) a plurality of anaerobic bacterial cells; b) seeding said intestinal cells in said microfluidic device so as to provide an intestinal cell layer having a barrier function; c) contacting said intestinal cells in said intestinal cell layer with said plurality of bacterial cells so as to allow at least some of said bacterial cells to adhere to said intestinal cells; and d) culturing said anaerobic bacteria without a anaerobic chamber. In one embodiment, said culturing of step d) is in the presence of some oxygen.

In one embodiment, the present invention contemplates a method of treating cells, comprising: a) providing, i) a microfluidic device; ii) a plurality of cells obtained from at least one parenchymal cell population; iii) a plurality of bacterium from at least one bacteria strain; and b) seeding said parenchymal cells in said microfluidic device so as to provide a parenchymal cell layer having an apical side and a basal side; and c) contacting said parenchymal cells with said plurality of bacterium so as to allow adherence of at least some of said bacteria to said parenchymal cells. In one embodiment, said at least some of said adhered bacterium provide bacteria colonies growing on said apical side of said parenchymal cell layer. In one embodiment, said at least some of said adhered bacterium create a biofilm on said apical side of said parenchymal cell layer. In one embodiment, said at least some of said contacted bacterium are extracellular to said parenchymal cells. In one embodiment, said extracellular bacterium are located in between said parenchymal cells. In one embodiment, said in between bacterium are located below the apical barrier of said parenchymal cells. In one embodiment, said at least some of said contacted bacterium become intracellular in said parenchymal cells. In one embodiment, said parenchymal cells are selected from the group consisting of healthy parenchymal cells and diseased parenchymal cells.

In one embodiment, the present invention contemplates a method of treating cells, comprising: a) providing, i) at least 2 microfluidic devices; ii) a plurality of cells from at least two parenchymal cell populations; iii) a plurality of bacterium from at least one strain of bacteria; and b) seeding said parenchymal cell population 1 in said first microfluidic device and seeding said parenchymal cell population 2 in said first microfluidic devices so as to provide a parenchymal cell layer having an apical side and a basal side; and c) contacting said parenchymal cells with said plurality of bacteria so as to provide bacteria colonies growing on said apical side of said parenchymal cell layer.

In one embodiment, the present invention contemplates a microfluidic device comprising parenchymal cells in contact with a plurality of at least one strain of bacteria. In one embodiment, said parenchymal cells are selected from the group consisting of healthy parenchymal cells and diseased parenchymal cells. In one embodiment, said parenchymal cells are epithelial cells. In one embodiment, said parenchymal cells are selected from the group consisting of cell lines, noncancerous cells, cancerous cells, primary cells, patient derived cells, cells having known genetic mutations, cells having unknown genetic mutations, and test cells. In one embodiment, said bacteria strain is selected from the group consisting of nonpathogenic bacteria and pathogenic bacteria. In one embodiment, said bacteria strain is selected from the group consisting of probiotic bacteria, nonprobiotic bacteria, disease associated bacteria, disease specific bacteria and test bacteria. In one embodiment, said at least some of said contacted bacteria are adhered to said parenchymal cells. In one embodiment, said bacteria form a biofilm. In one embodiment, said device further comprises at least two microchannels separated by a membrane having two sides, wherein membrane side 1 faced one microchannel and membrane side 2 faces the other microchannel. In one embodiment, said parenchymal cells at attached to said side 1 of said membrane. In one embodiment, said device further comprises endothelial cells attached to said side 2 of said membrane. In one embodiment, said endothelial cells are selected from the group consisting of cell lines, noncancerous cells, cancerous cells, primary cells, patient derived cells, cells having known genetic mutations, cells having unknown genetic mutations, and test cells.

In one embodiment, the present invention contemplates a microfluidic device comprising parenchymal cells in contact with a plurality of at least one strain of bacteria. In one embodiment, said parenchymal cells are selected from the group consisting of healthy parenchymal cells and diseased parenchymal cells. In one embodiment, said parenchymal cells are epithelial cells. In one embodiment, said parenchymal cells are selected from the group consisting of cell lines, noncancerous cells, cancerous cells, primary cells, patient derived cells, cells having known genetic mutations, cells having unknown genetic mutations, and test cells. In one embodiment, said bacteria strain is selected from the group consisting of nonpathogenic bacteria and pathogenic bacteria. In one embodiment, said bacteria strain is selected from the group consisting of probiotic bacteria, nonprobiotic bacteria, disease associated bacteria, disease specific bacteria and test bacteria. In one embodiment, said at least some of said contacted bacteria are colonies on top of said parenchymal cells. In one embodiment, said at least some of said contacted bacteria are adhered to said parenchymal cells. In one embodiment, said bacteria forming a biofilm. In one embodiment, said device further comprises at least two microchannels separated by a membrane having two sides, wherein membrane side 1 faced one microchannel and membrane side 2 faces the other microchannel. In one embodiment, said parenchymal cells at attached to said side 1 of said membrane. In one embodiment, said device further comprises endothelial cells attached to said side 2 of said membrane. In one embodiment, said endothelial cells are selected from the group consisting of cell lines, noncancerous cells, cancerous cells, primary cells, patient derived cells, cells having known genetic mutations, cells having unknown genetic mutations, and test cells.

In one embodiment, the present invention contemplates a method of treating intestinal cells, comprising: a) providing, i) a plurality of duplicate microfluidic devices; ii) a plurality of cells obtained from at least one intestinal cell population; iii) a plurality of bacterium from at least one bacteria strain; and iv) a test compound; and b) seeding said intestinal cells in said duplicate microfluidic devices so as to provide a intestinal cell layer having an apical side and a basal side; c)contacting said intestinal cells in some said duplicate microfluidic devices with said plurality of bacterium so as to allow adherence of at least some of said bacteria to said intestinal cells; and d) observing a readout in said duplicate microfluidic devices contacted with said bacteria compared to said duplicate microfluidic devices without said bacteria for indicating a potential therapeutic use of said test compound. . In one embodiment, said readout is a count of said adhered bacteria and said test compound reduces said bacteria count. In one embodiment, said at least some of said adhered bacterium provides bacteria colonies growing on said apical side of said intestinal cell layer. In one embodiment, said readout is a count of bacteria colonies and said test compound reduces said bacteria colonies. In one embodiment, said at least some of said adhered bacterium creates a biofilm on said apical side of said intestinal cell layer. In one embodiment, said readout is a reduction of the surface area covered by said biofilm. In one embodiment, said at least some of said contacted bacterium is extracellular to said intestinal cells. In one embodiment, said readout is a count of said extracellular bacteria and said readout reduces the number of extracellular bacteria. In one embodiment, said extracellular bacteria are located in between said intestinal cells. In one embodiment, said bacterium is located below the apical barrier of said intestinal cells. In one embodiment, said at least some of said contacted bacterium become intracellular in said intestinal cell. In one embodiment, said readout is a count of said intracellular bacteria and said readout reduces the number of said intracellular bacteria. In one embodiment, said intestinal cells are selected from the group consisting of healthy intestinal cells and diseased intestinal cells. In one embodiment, said bacteria strain is selected from the group consisting of pathogenic bacteria strain and a nonpathogenic bacteria strain.

In one embodiment, the present invention contemplates a method of identifying a probiotic bacteria strain, comprising: a) providing, i) a plurality of duplicate microfluidic devices; ii) a plurality of cells obtained from at least one intestinal cell population; iii) a plurality of bacterium from at least one candidate bacteria strain; and iv) a plurality of bacterium from at least one comparative bacteria strain; b) seeding said intestinal cells in said duplicate microfluidic devices so as to provide a intestinal cell layer having an apical side and a basal side; c) contacting said intestinal cells in some said duplicate microfluidic devices with said plurality of said candidate bacteria and contacting said intestinal cells in other said duplicate microfluidic devices with said plurality of said comparative bacteria so as to allow adherence of at least some of said bacteria to adhere to said intestinal cells; and d) observing a readout in said duplicate microfluidic devices contacted with said candidate bacteria compared to said duplicate microfluidic devices with comparative bacteria for identifying a probiotic bacteria strain.

In one embodiment, the present invention contemplates a method of identifying a therapeutic bacteria strain, comprising: a) providing, i) a plurality of microfluidic devices; ii) a plurality of cells from at least two intestinal cell populations; iii) a plurality of bacterium from at least one strain of bacteria; and b) seeding said intestinal cell population 1 in some of said microfluidic devices and seeding said intestinal cell population 2 in microfluidic devices so as to provide a intestinal cell layer having an apical side and a basal side; and c) contacting said intestinal cells with said plurality of bacteria so as to provide bacteria colonies growing on said apical side of said intestinal cell layer; and d) observing a readout in said duplicate microfluidic devices contacted with said bacteria strain on intestinal cell population 1 compared to said duplicate microfluidic devices with intestinal cell population 2 for identifying a therapeutic bacteria strain. In one embodiment, said intestinal cell populations are selected from the group consisting of healthy intestinal cells and diseased intestinal cells. In one embodiment, said intestinal cells are primary epithelial cells. In one embodiment, said intestinal cells are selected from the group consisting of cell lines, noncancerous cells, cancerous cells, primary cells, patient derived cells, cells having known genetic mutations, cells having unknown genetic mutations, and test cells. In one embodiment, said bacteria strain is selected from the group consisting of nonpathogenic bacteria and pathogenic bacteria. In one embodiment, said bacteria strain is selected from the group consisting of candidate probiotic bacteria, probiotic bacteria, nonprobiotic bacteria, disease associated bacteria, disease specific bacteria and test bacteria. In one embodiment, said at least some of said contacted bacteria are adhered to said intestinal cells. In one embodiment, said bacteria form a biofilm. In one embodiment, said device further comprises comprising at least two microchannels separated by a membrane having two sides, wherein membrane side 1 faced one microchannel and membrane side 2 faces the other microchannel. In one embodiment, said intestinal cells at attached to said side 1 of said membrane. In one embodiment, said device further comprises endothelial cells attached to said side 2 of said membrane. In one embodiment, said endothelial cells are selected from the group consisting of cell lines, noncancerous cells, cancerous cells, primary cells, patient derived cells, cells having known genetic mutations, cells having unknown genetic mutations, and test cells.

In one embodiment, the present invention contemplates a microfluidic device comprising intestinal cells in contact with a plurality of at least one strain of bacteria. In one embodiment, said intestinal cells are selected from the group consisting of healthy intestinal cells and diseased intestinal cells. In one embodiment, said intestinal cells are epithelial cells. In one embodiment, said intestinal cells are selected from the group consisting of cell lines, noncancerous cells, cancerous cells, primary cells, patient derived cells, cells having known genetic mutations, cells having unknown genetic mutations, and test cells. In one embodiment, said bacteria strain is selected from the group consisting of nonpathogenic bacteria and pathogenic bacteria. In one embodiment, said bacteria strain is selected from the group consisting of probiotic bacteria, nonprobiotic bacteria, disease associated bacteria, disease specific bacteria and test bacteria. In one embodiment, said at least some of said contacted bacteria are colonies on top of said intestinal cells. In one embodiment, said at least some of said contacted bacteria are adhered to said intestinal cells. In one embodiment, said bacteria form a biofilm. In one embodiment, said device further comprises at least two microchannels separated by a membrane having two sides, wherein membrane side 1 faced one microchannel and membrane side 2 faces the other microchannel. In one embodiment, said intestinal cells at attached to said side 1 of said membrane. In one embodiment, said device further comprises endothelial cells attached to said side 2 of said membrane. In one embodiment, said endothelial cells are selected from the group consisting of cell lines, noncancerous cells, cancerous cells, primary cells, patient derived cells, cells having known genetic mutations, cells having unknown genetic mutations, and test cells.

In one embodiment, the present invention contemplates a method of culturing, comprising: a) providing a fluidic device, wherein said microfluidic device comprises a first microchannel and a second microchannel separated by a membrane, said membrane comprising first and second sides, wherein said first side serves as a surface for said first microchannel and said second side serves as a surface for said second microchannel; b) said fluidic device further comprising first living cells attached to said first side of said membrane; c) introducing a fluid at a continuous flow rate into said first microchannel; d) beginning cyclic stretching of said membrane at a frequency level; e) stopping said fluid flow and said cyclic stretching for providing a static period comprising air introduced into said first microchannel for providing an air-liquid interface; and f) alternating said static period with combined steps c) and d) for providing enhancement of said living cells. In one embodiment, said method further comprises step e) further comprising flowing said fluid into said first microchannel for replacing said air by immersing said living cells in said fluid. In one embodiment, said living cells are intestinal cells. In one embodiment, said intestinal cells are a colon epithelial cell layer In one embodiment, said enhancement is an increase in percentage of Goblet cells in said colon epithelial cell layer In one embodiment, said step c) and step d) occur simultaneously for a time period. In one embodiment, said combined step c) and step d) extends for a twenty four hour time period. In one embodiment, said static period extends for a twenty-four hour time period. In one embodiment, said air-liquid interface of said static period extends for the first six hours of said 24 hour time period. In one embodiment, after said six hours of said air-liquid interface, said fluid is flowed into said first microchannel for said immersing said living cells for an 18 hour time period. In one embodiment, repeating step f) up to three times. It is not meant to limit the cycle to three times, indeed, additional repeats may be initiated, up to four times, or more. In one embodiment, said device is inoculated with one or more microorganisms capable of developing biofilms followed by culturing said first living cells and said microorganisms under conditions such that a biofilm is created by said microorganisms. In one embodiment, said first living cells are in a cell layer. In one embodiment, said microorganisms comprise bacterial cells. In one embodiment, said bacterial cells adhere to said first living cells. In one embodiment, said bacterial cells are selected from the group consisting of nonpathogenic bacterial strains and pathogenic bacterial strains. In one embodiment, said method further comprises measuring viability or proliferation of at least some of said microorganisms. In one embodiment, said method further comprises measuring the viability and/or function of said living cells. In one embodiment, said method further comprises introducing a drug into said device such that it contacts said biofilm.

In one embodiment, the present invention contemplates a method of culturing a microorganism, comprising: a) providing a fluidic device comprising first living cells; b) inoculating said device with one or more microorganisms capable of developing biofilms; and c) culturing said first living cells and said microorganisms under flow conditions such that a biofilm is created by said microorganisms. In one embodiment, said first living cells comprise intestinal cells. In one embodiment, said microorganisms comprise bacterial cells. In one embodiment, said device is a microfluidic device. In one embodiment, said flow conditions comprises introducing fluid at a flow rate.

In yet another embodiment, the present invention contemplates a method of identifying a probiotic bacteria strain, comprising: a) providing: i) a microfluidic device; ii) a plurality of living mammalian cells; and iii) a plurality of bacterial cells from a candidate bacteria strain; b) seeding said living mammalian cells in said microfluidic device so as to provide a cell layer having a barrier function; c) contacting said cell layer with said plurality of bacterial cells so as to allow at least some of said bacterial cells to adhere to said cell layer, so as to create contacted living cells; and d) detecting a change in said contacted living cells, thereby identifying said bacterial cells as probiotic. In one embodiment, said microfluidic device comprises first and second microchannels separated by a membrane, said membrane comprising first and second sides, wherein said first side serves as a surface for said first microchannel and said second side serves as a surface for said second microchannel. In one embodiment, the method further comprises introducing fluid at a flow rate prior to, during or after step c). In one embodiment, said living cells comprise intestinal cells. In one embodiment, said living cells comprise lung cells. In one embodiment, said change detected in step d) comprises an increase in said barrier function of said contacted intestinal cells. In one embodiment, said increase in barrier function is measured by an increase in the expression of one or more tight junction proteins. In one embodiment, said intestinal cells are primary epithelial cells. In one embodiment, said intestinal cells are patient derived cells. In one embodiment, the method further comprises the step e) adding said probiotic bacteria to a food product.

In yet another embodiment, the present invention contemplates a method of identifying a pathogenic bacteria strain, comprising: a) providing: i) a microfluidic device; ii) a plurality of living mammalian cells; and iii) a plurality of bacterial cells from a pathogenic bacteria strain; b) seeding said living mammalian cells in said microfluidic device so as to provide a cell layer having a barrier function; c) contacting said cell layer with said plurality of bacterial cells so as to allow at least some of said bacterial cells to adhere to said cell layer, so as to create contacted cells; and d) detecting a change in said contacted cells, thereby identifying said bacterial cells as pathogenic. In one embodiment, said living mammalian cells comprise intestinal cells. In one embodiment, said living cells comprise lung cells. In one embodiment, said microfluidic device comprises first and second microchannels separated by a membrane, said membrane comprising first and second sides, wherein said first side serves as a surface for said first microchannel and said second side serves as a surface for said second microchannel. In one embodiment, the method further comprises introducing fluid at a flow rate prior to, during or after step c). In one embodiment said change detected in step d) comprises a decrease in said barrier function of said contacted intestinal cells. In one embodiment, said decrease in barrier function is measured by a decrease in the expression of one or more tight junction proteins. In one embodiment, said intestinal cells are primary epithelial cells. In one embodiment, said intestinal cells are patient derived cells. In one embodiment, aid bacteria form a biofilm after step c).

In still another embodiment, the present invention contemplates a method of culturing anaerobic bacteria, comprising: a) providing: i) a microfluidic device; ii) a plurality of living mammalian cell; and iii) a plurality of anaerobic bacterial cells; b) seeding said living mammalian cells in said microfluidic device so as to provide a cell layer having a barrier function; c) contacting said cell layer with said plurality of bacterial cells so as to allow at least some of said bacterial cells to adhere to said cell layer; and d) culturing said anaerobic bacteria without a anaerobic chamber. In one embodiment, said culturing of step d) is in the presence of some oxygen. In one embodiment, said living mammalian cells comprise intestinal cells. In one embodiment, said living cells comprise lung cells. In one embodiment, a portion of said microfluidic device is oxygen permeable. In one embodiment, a portion of said microfluidic device is oxygen impermeable. In one embodiment, said microfluidic device comprises first and second microchannels separated by a membrane, said membrane comprising first and second sides, wherein said first side serves as a surface for said first microchannel and said second side serves as a surface for said second microchannel. In one embodiment, the method further comprises flowing a first fluid in said first microchannel and a second fluid in said second microchannel. In one embodiment, said first fluid is deoxygenated. In one embodiment, said second fluid is oxygenated. In one embodiment, said second fluid causes a gradient of oxygen in said first microchannel. In one embodiment, the fluid is culture media. In one embodiment, the gradient of oxygen is in said culture media. In one embodiment, the second microchannel is below said first microchannel and the oxygen in the media in the lower channel travels through the membrane into the upper channel.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Exemplary embodiments are illustrated in referenced figures. It is intended that the embodiments and figures disclosed herein are to be considered illustrative rather than restrictive.

FIG. 1A illustrates a perspective view of a microfluidic device with microfluidic channels in accordance with an embodiment (left) with a mirror image CAD illustration (right).

FIG. 1B illustrates an exploded view of a device in accordance with an embodiment, showing a microfluidic channel in a top piece and a microfluidic channel in a bottom piece, separated by a membrane.

FIG. 1C illustrates an exemplary schematic representation for one embodiment of a microfluidic intestine-on-chip: 1. Epithelial Channel; 2. Human Intestinal Epithelial Cells, e.g. Caco2, primary intestinal cells, cancer cells, etc.; 3. Vacuum Channel; 4. Membrane; 5. Human Intestinal Endothelial Cells e.g., HIMEC or iHIMEC, etc.; and 6. Vascular Channel. Designing and engineering the microenvironment allows us to recreate a “home away from home” for the cells within our Organ-Chips, including: Extracellular matrix and cell interactions; Cell shape and cytoarchitecture; Tissue-tissue interactions; Mechanical forces Dynamic system, including under flow and stretch, resident or circulating immune cells, etc.

FIG. 1D shows one embodiment of a microfluidic intestine-on-chip. In one embodiment a chip is comprised of at least two micro-channels (blue and pink channels) separated by a porous flexible-membrane. The material is functionalized with extracellular matrix and different types of cells are seeded into the two different channels. In one embodiment, of an Intestine On-Chip model, human endothelial cells are seeded in the bottom compartment (pink) and human epithelial cells in the top compartment (blue) to emulate the basic functioning unit of at least a portion of the intestine. Vacuum pressure can be applied to the side channels (gray) to mechanically stretch the membrane. Fluids can be continuously pumped through the channels to mimic shear forces, bring in nutrients, bring in bacteria, bring in test compounds and antibiotics, flush away wastes and provide effluent fluids for sampling. In another embodiment, of an Intestine On-Chip, resident immune cells are incorporated. In some embodiments, additional cells such as immune cells may be added. Environmental and Host Factors Implicated in GI Pathogenesis. Embodiments include additional cells such as intestinal mucosally derived cells or cultured cells such as found in the intestinal mucosa; resident immune cells, and sensory neurons in the epithelial channel.

FIGS. 1E-G shows immunofluorescent micrographs of immunostained embodiments of intestine on-chip derived from exemplary cell sources: Incorporating Patient-Derived Lamina Propria Immune Cells on-Chip. FIG. 1E Caco-2 BBE stained with Phalloidin—Actin (green), MUC2-Mucin 2 (purple), and DAPI—stained Nuclei colored blue.

FIG. 1F shows exemplary fluorescent micrographs of Primary Enteroids Muc2—Goblet Cells (pink), Lysozyme—Paneth Cells (green), and DAPI stained Nuclei colored blue.

FIG. 1G shows exemplary fluorescent micrographs of iPSC Organoids ZO-1—Tight Junction (green), E-cadherin (blue), Cdx2 stained Nuclei colored red.

FIG. 1H shows exemplary fluorescent micrographs (right) of embodiments of an intestine-on-chip (left) incorporating Lamina Propria Derived Immune Cells on the Intestine-Chip. Upper channel showing red tight junctions and blue colored DAPI staining of nuclei in the epithelial channel. Immune cells CD45+ colored purple, actin colored green and blue colored DAPI staining of nuclei in cells in the upper channel located below the epithelial cell layer. Endothelial cells showing actin in green, cell-cell junctions in red and blue colored DAPI staining of nuclei in the lower endothelial channel.

FIG. 2A-B shows an exemplary schematic of an open top microfluidic chip.

FIG. 2A illustrates a perspective view of a microfluidic open top device with microfluidic channels in accordance with an embodiment.

FIG. 2B illustrates an exploded view of an open top device in accordance with an embodiment, showing a microfluidic culture channel in a top piece and a microfluidic channel in a bottom piece, separated by a membrane.

FIGS. 3A-C shows exemplary micrographs of a bacteria strain, Moraxella catharalis (MC), that acts as an exacerbator and can induce biofilm formation on top of epithelial cells after infecting one embodiment of a microfluidic Airway On-Chip. 10⁶ CFU per chip. We showed that it is possible to infect the Airway Chip with bacterial pathogens for several days.

FIG. 3A shows exemplary MC ATCC bacterial strain co-cultured with epithelial cells, immunostained and colored green.

FIG. 3B shows exemplary MC clinical isolate bacterial strain. MOI 10 at 24 hours post-infection (hpi) of immunostained epithelial cells, and colored green.

FIG. 3C shows an exemplary confocal immunostained fluorescent micrograph including Z-stacks bands across the top and along the right hand side of the image, pink colored epithelial junction marker, green colored bacteria and blue DAPI stained nuclei. Respiratory pathogens involved in exacerbation include Moraxella catharallis. The upper (or right side on the side bar) part of the Z-stacks represent apical regions then down through the cells to the basil regions at the bottom of bar (or left side of the side bar). These Z-stacks indicate that bacterium are intracellularly located. Therefore, Moraxella catharallis bacteria are found inside the epithelial cells following infection of a microfluidic Airway Chip. This exemplary confocal image of the infected Airway chip shows intracellular staining (green). For comparison, transwells infected with 10⁶ CFU per transwell, at a MOI of 10, have no observable immunostained bacteria.

FIG. 4A shows an exemplary contemplated Experimental Plan and Timeline. Day 0: Seed Chips: Bottom Channel: HUVEC. Top Channel: Caco-2 epithelial cells, however any source of intestinal epithelial cell may be used. Day 1: Connect to flow. Day 3: Inoculation and Readouts: Imaging; Permeability; Bacterium Titer; etc. Day 8: Inflammatory Challenge. Readouts: Imaging; Permeability; Cytokines; Bacterium Titer; etc. Day 8+4 hrs: Readouts: Imaging; Permeability; Cytokines; Bacterium Titer; etc. Day 8+14 hrs: Readouts: Permeability; Cytokines; Effluent Titer; etc. Day 8+14 hrs: Collect Endpoints: RNA; Immunofluorescence; Adherent Titer; etc.

FIG. 4B-E demonstrates an inflamed Intestine on-Chip comprising bacteria that has weakened barrier function and a reduction in epithelial ‘villus’ heights and a significant cytokine secretion into effluent fluid, exemplary IL-6, IL-8, IL-12, and MIP1-alpha, in response to inflammatory challenge e.g. +TNF-α/IL-1β. Upper epithelial boundary (yellow); Tight junction immunostained (red); cell-cell junctions (green) and nuclei colored blue. 40×.

FIG. 4B shows an exemplary healthy Caco-2 epithelial cell layer. Avg. Tissue Height 157+/−1.5 μm.

FIG. 4C shows an exemplary inflamed and significantly thinner Caco-2 epithelial cell layer on chip after bacterial were added. Avg. Tissue Height 84+/−11 μm. FIG. 4D shows exemplary secretion of IL-6 (left) IL-8 (right).

FIG. 4E shows exemplary secretion of IL-12 (left) MIPlalpha (right).

FIG. 5 shows exemplary contemplated experimental Plan and Timeline. Day 0: Seed Chips. Bottom Channel: HUVEC, however any source of endothelial cells are contemplated for use. Top Channel: Caco-2, however any source of epithelial cells including but not limited to those as described herein, are contemplated for use. Day 1: Connect to flow. Readouts: Imaging; etc. Day 3: Inoculation and Readouts: Imaging; Permeability; Bacterium Titer; etc. Day 8: Inflammatory Challenge. Readouts: Imaging; Permeability; Cytokines; Bacterium Titer; etc. Day 8 +4 hrs: Readouts: Imaging; Permeability; Cytokines; Bacterium Titer; etc. Day 8 +14 hrs: Readouts: Permeability; Cytokines; Effluent Titer; etc. Day 8 +14 hrs: Collect Endpoints: RNA; Immunofluorescence; Adherent Titer; etc.

FIGS. 6A-B shows exemplary real time imaging after infection of one embodiment of an Airway Chip with bacteria P. aeruginosa infection on chip. Both pseudomonas strains, wild-type (WT) and mutant, form micro-colonies/aggregates on airway chip. Bacterial inoculum is plated and CFU counted to ensure target MOI. Images are acquired at 24 hpi. FIG. 46A PA 5919-WT. FIG. 6B PA 5890-Mutant. Left: Alexa fluor 488—anti-P. aeruginosa (Pa) antibody. Central: Bright-field. Right: Alexa fluor 488—anti-P. aeruginosa (Pa) antibody-Bright-field.

FIGS. 7A-C shows exemplary immunofluorescence, with Z-stacks or a side view, after infection of one embodiment of an Airway Chip with bacteria P. aeruginosa infection on chip. Pseudomonas establishes an intracellular niche as well as forming extracellular micro-colonies on the epithelial cell surface. Z-stacks are shown as a bar across the top (to the right of the 24 h label, and the down the right side of the micrographs. The upper (or right side on the side bar) part of the Z-stacks represent apical regions then down through the cells to the basil regions at the bottom of bar (or left side of the side bar). These Z-stacks indicate that bacterium are intracellularly located. FIG. 7A PA 5919-WT. Actin (red); Pa (green); DAPI (blue). FIG. 7B PA 5890-Mutant. Actin (pink); Pa (green); DAPI (blue). Images are acquired at 24 hpi. FIG. 7C shows a confocal immunofluorescent micrograph side view of a cell layer infected with P. aeruginosa in a microfluidic airway chip, 24 hours post infection. Actin (pink); Pa (green); DAPI (blue). Bacterial aggregates on apical surface as well as intracellular bacteria are observed.

FIGS. 8A-C shows exemplary mucociliary activity photographed in bright field on one embodiment of a Pseudomonas infection on chip. Micrographs represent one image from a video of cilia beating on-chip. FIG. 8A Non-infected control microfluidic chip image representing beating cilia. FIG. 8B PA 5890-Mutant infected microfluidic chip image representing loss of beating cilia. FIG. 8C PA 5919-WT microfluidic chip image also representing a loss of beating cilia.

FIGS. 9 shows an exemplary comparison of cilia beating frequency (CBF) between Pseudomonas strains in one embodiment of a Pseudomonas infection on chip. Images from a video of epidermal cells' cilia beating on-chip are quantitatively evaluated showing that both wild type and mutant strains has altered cilia beating frequency compared to controls without added bacteria.

FIGS. 10 an exemplary comparison of cellular cilia coverage after infection with Pseudomonas strains in one embodiment of a Pseudomonas infection on chip. Mutant (increases) and WT (decreases) show significant differences in density compared to controls.

FIG. 11 shows an exemplary Bacterial adherence on chip in one embodiment of a microfluidic airway epithelia. P. aeruginosa WT (MB5980) and mutant (MB5919) strains adhere to airway epithelium at similar rates. Unpaired t-tests p=0.0641. N=3.

FIGS. 12A-B shows an exemplary Imipenem (Merck compound) effects on P. aeruginosa infection. FIG. 12A shows exemplary Imipenem (Merck compound) effects on P. aeruginosa infection in a Transwell culture. FIG. 12B Imipenem treatment reduces total bacterial counts via bacterial killing in one embodiment of a P. aeruginosa infection on chip. Two-way ANOVA with Dunnett's post-test **<0.05, **<0.001 (compared to untreated).

FIGS. 13A-C shows exemplary Imipenem (Merck compound) effects on P. aeruginosa infection, WT vs. mutant, on airway cells in Transwells. FIG. 13A shows exemplary Imipenem treatment. FIG. 13B shows exemplary Carbenicillin treatment. FIG. 13C shows exemplary Tetracycline treatment. Two-way ANOVA with Dunnett's post-test **<0.05, **<0.001, ***<0.0001 (compared to untreated).

FIGS. 14A-C shows exemplary Real time imaging of Imipenem effects on P. aeruginosa infection on one embodiment of a PA 5919 WT Pseudomonas infection on chip. FIG. 14A untreated (noninfected) control. FIG. 14B 50 μg/ml. FIG. 14C 500 ug/m. PA 5919 WT 24 hpi. Left: Alexa fluor 488—anti-P. aeruginosa (Pa) antibody. Central: Bright-field. Right: Alexa fluor 488—anti-P. aeruginosa (Pa) antibody-Bright-field.

FIGS. 15A-C shows exemplary Real time imaging of Imipenem effects on P. aeruginosa infection on one embodiment of a PA 5890 Mutant 24 hpi Pseudomonas infection on chip. FIG. 15A untreated (noninfected) control. FIG. 15B 50 μg/ml. FIG. 15C 500 μg/m. PA 5890 Mutant 24 hpi. Left: Alexa fluor 488—anti-P. aeruginosa (Pa) antibody. Central: Bright-field. Right: Alexa fluor 488—anti-P. aeruginosa (Pa) antibody-Bright-field.

FIG. 16 shows an exemplary secretion of Human β-Defensin 2 post P. aeruginosa infection on one embodiment of a microfluidic Airway Chip. For comparison, HBD-2 protein (pg/ml) are measured in control chips; after WT P. aeruginosa; and a mutant strain of P. aeruginosa are tested for Human β-Defensin 2 secretion in apical wash, 24 hpi. Unpaired t-test, **<0.05. N=2.

FIGS. 17A-D shows exemplary apoptosis via TUNEL staining at 24 h post infection. Apoptotic, TUNEL+, (pink); nuclei, DAPI+, (blue). FIG. 17A uninfected; FIG. 17B Pa infected; FIG. 17C staurosporin treatment. Staurosporin refers to an ATP-competitive kinase inhibitor. FIG. 17D DNAse I treatment. DNAse I refers to an endonuclease that nonspecifically cleaves DNA to release di-, tri- and oligonucleotide products with 5′-phosphorylated and 3′-hydroxylated ends. DNase I acts on single- and double-stranded DNA, chromatin and RNA:DNA hybrids.

FIG. 18A-B shows exemplary cellular histology and cytokine secretion demonstrating human primary resident immune cells retain in vivo phenotype. Readouts include but are not limited to morphology and biomarkers; secreted cytokines; and permeability. Ulcerative colitis (UC) patient immune cells derived from regions of non-inflamed (niLP) and inflamed lamina propria (iLP) retain their inflammatory phenotype in the Intestine-Chip. Showed Increased pro-inflammatory cytokines secretion and weaker barrier function.

FIG. 18A shows exemplary colored immunofluorescent micrographs of immunostained cells—upper channel—epithelium and also in the upper channel—immune cells underlying epithelium. Lower channel—endothelium.

FIG. 18A shows exemplary IL-6, IL-8 secretion and a comparison of permeability between microfluidic chips comprising no LP vs. niLP vs. iLP.

FIG. 19A-B shows exemplary Lamina Propria Immune Cells Respond to Steroidal Treatment on-Chip. Study demonstrates ability to assess responses to anti-inflammatory treatments in ulcerative colitis patients Prednisone (10 μM-10X clinically relevant dosage) suppresses inflammatory responses before and after challenge with the gram-positive endotoxin, PAM2CSK4.

FIG. 20 shows exemplary immune cell recruitment in the Intestine-Chip. Recapitulation of the tissue microenvironment, including but not limited to the epithelial-endothelial interface, extra cellular matrix, under flow, enables immune recruitment studies under physiological relevant conditions. Upper, shows an illustration of one end of a microfluidic device comprising PBMCs (green)/HIMECs (red) shown as a colored florescent micrograph of immunostained cells, lower, of the area outlined by the dotted line.

FIG. 21A-B shows exemplary Intestine on-Chips were generated from patient biopsies enabling the study of, i.e. embodiments of, disease mechanisms and drug efficacy testing, such as an Enteroid-Derived Primary Intestine-Chip.

FIG. 21A illustrates one embodiment of a microfluidic device, e.g. a two-channel chip. Top channel—Intestinal Epithelial cells derived from Intestinal Organoids. Bottom channel—Intestinal microvascular endothelium (HIMEC). Left, bright filed images of cells seeded onto chips. Right images: immunostained actin shown in red, DAPI stain shown in blue.

FIG. 21B shows exemplary bright-field micrograph images of cells growing under flow on days 4, 6 and 12 compared to cells grown in static cultures, i.e. no flow, as labeled.

FIG. 22A-C shows examples of intestinal cell sources for seeding onto microfluidic intestine on-chip devices as described herein.

FIG. 22A shows photographic images of one example of whole tissue obtained from a bowel (colon) resection, left, and an example of whole tissue obtained from a biopsy obtained by endoscopy, right.

FIG. 22B shows micrograph images of one example of dissociated cells at the beginning of culture, right, obtained (derived) from a biopsy of crypt tissue, left.

FIG. 22C shows an example of cells differentiated from stem cells as ex-vivo cultures in matrigel, starting from media comprising+Wnt3A, EGF, Noggin, R-spondin (WENR), which may be in cell culture dishes or on chip. Left to right, images show 3 day, 5 day, 7 day, and 10 day cultures. L-luminal, B-basal.

FIG. 23 illustrates an exemplary timeline for seeding one embodiment of an intestine on chip with exemplary images of cells after seeding and attachment on Day 0, after culturing on Day 4 and Day 8. Both apical cell images (top micrograph) and basal cell images (bottom micrograph) are shown. As an example, chips are coated the Day before (Day −1) seeding cells on Day 0, showing images on chips seeded in the apical channel with a suspension of colonoids cells and cHIMEC cells prepared on plates seeded in the basal channel. Flow is connected at Day 1, cells are maintained in this example until Day 8.

FIG. 24A-C shows examples of immunostained images demonstrating polarization and differentiation of the colonic epithelium. Absorptive enterocytes and Goblet cells in the Colon-Chip show in vivo-like apical polarization. Recreating Tissue-tissue interfaces dramatically accelerates epithelial barrier formation on the Colon-Chip.

FIG. 24A shows exemplary 3D reconstruction of one embodiment of an immunofluorescent image of a colon epithelium on-Chip. F-actin immunostaining shown in pink and nuclear DAPI staining shown in blue. Right panel is an enlarged image of one area of the right panel.

FIG. 24B shows exemplary images from left to right, basal plane, middle plane and apical plane matching the dotted line areas shown in a side-view in FIG. 24C. Villin (Absorptive)—green. Muc2 (Goblet)—pink. DAPI (Nuclei)—blue.

FIG. 25A-B shows examples of epithelial barrier formation on the Colon On-Chip. The presence of colon-specific microvascular cells improves the attachment of the epithelial tissue to the chip resulting in more robust monolayer with increased undulations characteristic of the rugged colonic morphology in-vivo. The endothelium also significantly improves the formation of epithelial barrier function on-Chip at early time-points indicating a key role in the maturation of absorptive enterocytes.

FIG. 25A shows exemplary effects of endothelium upon barrier formation as bright field microscopic images. Upper row of bright filed images+(plus) endothelium in basal channel; lower row of images−(minus) endothelium, on Day 1, Day 4, and Day 8.

FIG. 25B shows measurements of exemplary apparent permeability (Papp(cm/s*10-7) of one embodiment of a colon-chip, with and without endothelium. The intestinal barrier forms faster and appears tighter with endothelial cells cultured in the lower channel.

FIG. 26A-B shows exemplary microenvironment characteristics of one embodiment of a microfluidic Colon On-Chip: Goblet Cells grow in abundance as shown in intestinal cells derived (obtained) from at least 2 different donors.

FIG. 26A comparison of two different exemplary donors: Immunostained Donor 1, left panels; Donor 2, right panels. Muc2 (Goblet cells) colored pink, DAPI (Nuclei) colored blue. −HIMEC upper row; +HIMEC lower row.

FIG. 26B Comparison of goblet cell abundance in intestinal cells cultured from two different exemplary donors as goblet cell populations: Muc2+ cells/DAPI+ cells percentage-plus (left blue bars) and minus (right grey bars) endothelium.

FIG. 27A-D shows exemplary colored immunofluorescent micrograph images of Colon-Chip Epithelial Differentiation of cells from one exemplary donor, immunostained for specified cell characteristics. FIG. 27A shows immunostaining for Villin (Absorptive) green and DAPI (Nuclei) blue; FIG. 27B shows immunostaining for ZO-1 (Tight Junctions) yellow and DAPI (Nuclei) blue; FIG. 27C shows immunostaining for ChrA (Enteroendocrine) turquoise and DAPI (Nuclei) blue; and FIG. 27D shows immunostaining for MUC2 (Goblet) pink and DAPI (Nuclei).

FIG. 27E-G shows exemplary colored immunofluorescent micrograph images of immunostained epithelial cells on chip, derived from patient-specific sources of cells, where cells are undergoing epithelial differentiation and formation of tight junctional networks are similar with and without the presence of endothelial cells, i.e. an endothelial tissue interface.

FIG. 27E shows immunostaining for Villin (Absorptive) green and DAPI (Nuclei) blue; FIG. 27F shows immunostaining for ChrA (Enteroendocrine) turquoise and DAPI (Nuclei) blue; and FIG. 27G shows immunostaining for ZO-1 (Tight Junctions) yellow and DAPI (Nuclei) blue.

FIG. 28A-B shows exemplary immunofluorescent micrograph images of Enteroendocrine Cell Localization on the Colon-Chip. In vivo-like tissue localization of chromogranin positive, enteroendocrine cells. Preliminary data suggests EEC cell differentiation may be depend on epithelial tissue height—reflecting in vivo-like crypt organization.

FIG. 28A shows exemplary, left to right panels, DAPI Nuclei—blue; ZO-1 Tight Junctions—yellow; and ChrA (enteroenteroendocrine cells (EECs)-turquoise.

FIG. 28B shows exemplary merged images (FIG. 28A) with an area outlined by dotted lines (left panel) enlarged in the right panel.

FIG. 29 shows exemplary goblet cells immunostained then colored pink on Day 4 vs. Day 8. Muc2+ pink (Goblet cells) Healthy donor (left) and UC donor (inflamed), right. E-cadherin+ (Tight Junctions)—yellow and Hoechst stain (Nuclei)—blue. Intestine On-Chip seeded with intestinal cells obtained from a patient with ulcerative colitis (UC) as diseased (right panel) compared to goblet cells in normal (left panel). A clear difference in goblet cell population is observed between the epithelium derived from healthy and UC diseased patients.

FIG. 30A-C illustrates and shows that physiological oxygen concentrations improves Colon-Chip tissue morphology and increases the rate of apoptosis, an important feature of epithelial homeostasis.

FIG. 30A illustrates an exemplary hypoxic luminal channel where O₂ from the lower channel is depleted by the endothelial cells and basal end of the epithelial cells so that the upper apical region of the epithelial layer is hypoxic where anaerobic bacteria may grow. O₂ Concentration is represented on the right side in blue, where the wider blue area represents a more oxygenated area than the upper thinner blue area.

FIG. 30B-C demonstrates an exemplary effect of physoxia on Colon On-Chip cellular morphology.

FIG. 30B shows exemplary bright-field micrographs of cells on chips under 21% O₂ (left) vs. 5% O₂ (right).

FIG. 30C shows exemplary apoptosis of cells compared between 2 chips, were apoptotic cells are colored green (TUNEL+)/DAPI (blue) under 21% O₂ (upper) vs. 5% O₂ (lower). Physiological oxygen concentrations (physoxia), around 5% O₂, improves Colon-Chip tissue morphology and increases the rate of cellular apoptosis, a feature of healthy epithelial homeostasis.

FIG. 31A-B shows exemplary commensal bacterial strains; Lactobacillus rhamnosus GG (LGG) and Clostridia (C.) symbiosum on a microscope slide. Samples were obtained from inoculum before mixing the populations 1:1 and introducing them into one embodiment of a Colon-Chip. Physoxia microenvironment supports Colon On-Chip epithelium. FIG. 31A shows exemplary LGG bacteria (white). FIG. 31B shows exemplary C. symbiosum bacteria (white).

FIG. 32 shows exemplary morphology of Colon-Chip in normal (21% O₂) vs. physoxia microenvironment (5% O₂). 21% O₂ (upper row showing micrographs of cells) vs. 5% O₂ (lower row showing micrographs of cells) over time, left to right, Day 0, Day 3, Day 4, Day 7 and Day 7 plus C. symbiosum.

FIG. 33 shows an exemplary embodiment of a Colon On-Chip co-culture with anaerobic commensal bacteria. A physoxia microenvironment (5% Oxygen) on the Colon-Chip supports anaerobic commensal bacteria. Left image shows an illustration of a micofluidic device, florescent micrographs show immunostained C. symbiosum (green) cells obtained from the central upper channel (dotted lines) comparing chips having 21% O₂+C. symbiosum (middle panel) vs. 5% O₂ C. symbiosum (right). DAPI stained nuclei of epithelial cells shown in blue.

FIG. 34 shows an exemplary epithelial interaction with LGG. Left to right, a Brightfield image of a cross-section of one embodiment of a microfluidic intestine on-chip showing cells growing on-chip; middle shows a fluorescent micrograph of immunostained cell-cell junctions (green) and immunostained bacteria (purple) representing the area outlined in white dotted line (left); right paned shows a Scanning electron micrograph (SEM) of bacteria attached to epithelial cells (upper right enlarged micrograph showing individual bacterium). Thus, one embodiment of an Intestine On-Chip has epithelial microbiome interactions.

FIG. 35 shows an exemplary embodiment of an iPSC derived Intestine On-Chip. Epithelium derived from 3D intestinal organoids (illustrated in upper panel), as differentiating cells cultured in dishes then seeded onto chips or differentiated on chip then disassociated and used to seed a second chip. Differentiation of major intestinal cell-types in and maturation of a 3D villus-like morphology on-Chip. Applications include but are not limited to: Potential for differentiation of other patient-matched tissue cell-types particularly of immune lineage; and Applications in precision medicine and disease modeling.

FIG. 36 shows an exemplary embodiment of fluid loading into channels on chips. In other embodiments syringes may be used for effluent collection, depending upon which chip port is connected via tubing to the syringe.

FIG. 37 illustrates embodiments comprising an exemplary Intestine On-Chip enabling microbiome studies. Co-culture strategy of intestinal cells with commensal microbes. Intestine-Chip enables the study of interaction of human microbiome with human intestinal tissue.

FIG. 38A-B Micrographs showing shapes of Commensal Bacteria Strains For Potentially Providing Probiotic Activity In One Embodiment Of A Microfluidic Intestine On-Chip. FIG. 38A Micrograph of Lactobacilli bacteria. FIG. 38B Micrograph of Bifidobacteria, a type of lactic acid bacteria, is a rod-shaped bacterium frequently observed with branches, e.g. Y shaped.

FIG. 39A-B shows exemplary Epithelial Morphology in representative brightfield images of Intestine On-Chip. FIG. 39A Control Chip (no bacteria added), Day 8. FIG. 39B Chip treated with +TNF-α/IL-1β, Day 8 (no bacteria added).

FIG. 40A-B shows exemplary Epithelial Morphology in representative bright-field images of Intestine On-Chip, untreated. FIG. 40A Control Chip (no bacteria added), Day 8. FIG. 40B Chip treated with LGG1, Day 8.

FIG. 41A-B shows exemplary Epithelial Morphology in representative bright-field images of Intestine On-Chip, treated. FIG. 41A Control Chip (no bacteria added), Day 8. FIG. 41B Chip treated with +LGG1 in the presence of +TNF-α/IL-1β, Day 8.

FIG. 42A-B shows exemplary Epithelial Morphology in representative bright-field images of Intestine On-Chip, untreated. FIG. 42A Control Chip (no bacteria added), Day 8. FIG. 42B Chip treated with +LGG2, Day 8.

FIG. 43A-B shows exemplary Epithelial Morphology in representative bright-field images of Intestine On-Chip, treated. FIG. 43A Control Chip (no bacteria added), Day 8. FIG. 43B Chip treated with +LGG2 in the presence of +TNF-α/IL-1β, Day 8.

FIG. 44A-B shows exemplary Lactobacilli Strains that Stably Colonized the Intestine-Chip in part shown by exemplary read-outs of bacterial titration and apparent permeability. FIG. 44A Bacterial Titration (counts mL⁻¹) at Day 3 and Day 4 compared to adherent counts at Day 4. FIG. 44B Apparent Permeability demonstrating improved barrier function after either Lactobacilli LGG1 and LGG2 infection.

FIG. 45A-C shows exemplary Lactobacilli Strains (LGG1 And LGG2) Modulate Epithelial Cytokine Responses. Duplicate chips were cultured with or without stimulatory TNF-α/IL-1β, then with or without one of the LGG test strains. FIGS. 45A IL-6 (pg/ml) Time Post Treatment 4 hr (left) 14 hr (right). FIGS. 45B IL-10 (pg/ml) Time Post Treatment 4 hr (left) 14 hr (right). FIGS. 45C IFN-gamma (pg/ml) Time Post Treatment 4 hr (left) 14 hr (right).

FIG. 46A-B shows exemplary Lactobacillus modulation of the IL-6 Epithelial Cytokine Response. FIGS. 46A shows IL-6 Cytokine Production +LGG1. FIGS. 46B shows IL-6 Cytokine Production +LGG2.

FIG. 47A-D shows exemplary Lactobacillus upregulated Endothelial Selectin Expression. Gene Expression Measured by qPCR. Control (no bacteria); Lactobacilli Strains LGG1 vs. LGG2 with or without TNF-α/IL-1β. Log2 (fold-challenge). FIG. 47A ICAM. FIG. 47B MADCAM. FIG. 47C PECAM. FIG. 47D VECAM.

FIG. 48A-B shows exemplary Lactobacillus upregulated Endothelial Selectin Expression. FIG. 48A Immunofluorescence of Endothelial ICAM Selectin Expression. FIG. 48B Image Quantification of ICAM Expression. Control (no bacteria); Percent cell area.

FIG. 49A-B shows exemplary representative bright-field (FIG. 49A) and immunofluorescent images (FIG. 49B) of Intestine-Chip cross-sections. FIG. 49A bright-field micrograph Cross-Section of a +LGG1 contacted chip. FIG. 49B immunofluorescent micrograph of a +LGG2 contacted chip, green LGG2, red LGG2 and nuclei DAPI colored blue.

FIG. 50A-B shows exemplary Scanning Electron Micrographs of LGG on the Intestine-Chip. FIG. 50A bar=5 μm. FIG. 50B higher power image. bar=1 μm.

FIG. 51A-B shows exemplary Epithelial Morphology in representative bright-field images of Intestine On-Chip. FIG. 51A Control Chip (no bacteria added), Day 8. FIG. 51B Chip treated with +Bif1, Day 8.

FIG. 52 shows an exemplary Aerotolerant Bifidobacteria Stably Colonize the Intestine-Chip. Bacterial Titration. Viable, aerotolerant bifidobacterial strains colonized the Caco-2 Intestine-Chip and could be detected in plate Titers.

FIG. 53A-B shows exemplary Bifidobacterial modulation of Epithelial Morphology. Representative brightfield images of Intestine-Chip, untreated. FIG. 54A Control Chip (no bacteria added), Day 8. FIG. 54B Chip treated with +Bif1, Day 8.

FIG. 54A-B shows exemplary Bifidobacterial modulation of Epithelial Morphology. Representative brightfield images of Intestine-Chip, treated. FIG. 55A Control Chip Chip treated with +TNF-α/IL-1β (no bacteria added), Day 8. FIG. 55B Chip treated with +Bif1+TNF-α/IL-1β, Day 8.

FIG. 55A-B shows exemplary Bifidobacterial modulation of Epithelial Morphology. Representative brightfield images of Intestine-Chip, untreated. FIG. 56A Control Chip treated with +TNF-α/IL-1β (no bacteria added), Day 8. FIG. 56B Chip treated with +Bif2+TNF-α/IL-1β, Day 8.

FIG. 56A-B shows exemplary Bifidobacterial modulation of Epithelial Morphology. Representative brightfield images of Intestine-Chip, treated. FIG. 57A Control Chip treated with +TNF-α/IL-1β (no bacteria added), Day 8. FIG. 57B Chip treated with +Bif2+TNF-α/IL-1β, Day 8.

FIG. 57A-B shows exemplary Bifidobacterial modulation of Epithelial Barrier Function that does not appear to significantly change by day 8-post infection. FIG. 58A Apparent Permeability over time between control (no bacteria), Bif1 and Bif2. FIG. 58B Comparative apparent Permeability on Day 8 (14 hours post treatment).

FIG. 58A-D shows exemplary secreted cytokines. Bifidobacterial modulation of epithelial cytokine responses. FIG. 59A Secreted IL-6 (pg/ml) post-treatment 4 hr (top graph) post-treatment 14 hr (bottom graph). FIG. 59B Secreted IL-8 (pg/ml) post-treatment 4 hr (top graph) post-treatment 14 hr (bottom graph). FIG. 59C Secreted IL-10 (pg/ml) post-treatment 4 hr (top graph) post-treatment 14 hr (bottom graph). FIG. 59D Secreted IFN-gamma (pg/ml) post-treatment 4 hr (top graph) post-treatment 14 hr (bottom graph).

FIG. 59A-D shows exemplary Bifidobacterial modulation of epithelial tight junctions. Bif1 vs. Bif2 in relation to control (no bacteria); with and without TNF-α/IL-1β. Gene Expression Measured by qPCR. FIG. 60A shows exemplary expression of Claudin 1. FIG. 60B shows exemplary expression of Claudin 2. FIG. 60C shows exemplary expression of Claudin 3. FIG. 60D shows exemplary expression of Occludin.

FIG. 60A-C shows an exemplary Bifidobacterial modulation of epithelial inflammatory processes. Bif1 vs. Bif2 in relation to control (no bacteria); with and without TNF-α/IL-1β. Gene Expression Measured by qPCR. FIG. 61A shows exemplary expression of IL-8. FIG. 61B shows exemplary expression of S100A9. FIG. 61C shows exemplary expression of epithelial-cell—derived cytokine thymic stromal lymphopoietin (TSLP).

FIG. 61A-D shows an exemplary Bifidobacterial modulation of endothelial selectin expression. Bif1 vs. Bif2 in relation to control (no bacteria); with and without TNF-α/IL-1β. Gene Expression Measured by qPCR. FIG. 62A shows exemplary expression of ICAM. FIG. 62B shows exemplary expression of MADCAM. FIG. 62C shows exemplary expression of PCEAM. FIG. 28D shows exemplary expression of VECAM.

FIG. 62A-B shows exemplary immunofluescent immunostained micrograghs and charts demonstrating that Bifidobacteria downregulated endothelial selectin expression. FIG. 63A Immunofluorescence of ICAM Selectin Expression. Control (no bacteria); +Bif1+Bif2; with or without TNF-α/IL-1β. FIG. 63B Quantification of ICAM Expression; Percent cell area. Control (no bacteria); +Bif1 Bif2 NOT LGG1 vs. LGG2 as labeled on slide with or without TNF-α/IL-1β.

FIG. 63 shows exemplary bacterial titration over time collected from one embodiment of a microfluidic intestine on-chip. Viable Bifidobacteria were not Recovered from one embodiment of an Intestine On-Chip.

FIG. 64 shows exemplary Bifidobacteria strain colony streaks on brain heart infusion agar. Bif1 strain above black line. Bif2 strain below black line.

FIG. 65 shows exemplary LGG1 vs. LGG2 bacterial modulation of cytokine response as a profile of cytokine production.

FIG. 66A-B shows exemplary LGG1 vs. LGG2 bacterial modulation of cytokine response. Control (no bacteria); LGG1 vs. LGG2; with or without TNF-α/IL-1β. FIG. 66A IL-8 cytokine production at 4 hrs post-treatment. FIG. 66B IL-8 cytokine production at 4 hrs post-treatment.

FIG. 67A-B shows exemplary LGG1 vs. LGG2 bacterial modulation of cytokine responses between controls (no bacteria); LGG1 vs. LGG2; with or without TNF-α/IL-1β. FIG. 67A INF-γ (IFN-gamma) cytokine production at 4 hrs post-treatment. FIG. 67B INF-γ cytokine production at 14 hrs post-treatment.

FIG. 68A-B shows exemplary Bacterial Modulation of Cytokine Response. Control (no bacteria); LGG1 vs. LGG2; with or without TNF-α/IL-1β. FIG. 68A IL-10 cytokine production at 4 hrs post-treatment. FIG. 68B IL-10 cytokine production at 14 hrs post-treatment.

FIG. 69A-B shows exemplary LGG1 vs. LGG2 bacterial modulation of cytokine responses between controls (no bacteria); LGG1 vs. LGG2; with or without TNF-α/IL-1β. FIG. 69A IL-12/IL-23p40 cytokine production at 4 hrs post-treatment. FIG. 69B IL-12/IL-23p40 cytokine production at 14 hrs post-treatment.

FIG. 70A-B shows exemplary LGG1 vs. LGG2 bacterial modulation of cytokine responses between controls (no bacteria); (no bacteria); LGG1 vs. LGG2; with or without TNF-α/IL-1β. FIG. 70A IL-12p70 cytokine production at 4 hrs post-treatment. FIG. 70B IL-12p70 cytokine production at 14 hrs post-treatment.

FIG. 71A-B shows an exemplary Bifidobacterial modulation of cytokine responses between controls (no bacteria); Bif1 vs. Bif2; with or without TNF-α/IL-1β. FIG. 71A IL-6 cytokine production at 4 hrs post-treatment. FIG. 71B IL-6 cytokine production at 14 hrs post-treatment.

FIG. 72A-B shows an exemplary Bifidobacterial modulation of cytokine responses between controls (no bacteria); Bif1 vs. Bif2; with or without TNF-α/IL-1β. FIG. 72A IL-8 cytokine production at 4 hrs post-treatment. FIG. 72B IL-8 cytokine production at 14 hrs post-treatment.

FIG. 73A-B shows an exemplary Bifidobacterial modulation of cytokine responses between controls (no bacteria); Bif1 vs. Bif2; with or without TNF-α/IL-1β. FIG. 73A IL-10 cytokine production at 4 hrs post-treatment. FIG. 73B IL-10 cytokine production at 14 hrs post-treatment.

FIG. 74A-B shows an exemplary Bifidobacterial modulation of cytokine responses between controls (no bacteria); Bif1 vs. Bif2; with or without TNF-α/IL-1β. FIG. 74A IL-12/IL-23p40 cytokine production at 4 hrs post-treatment. FIG. 74B IL-12/IL-23p40 cytokine production at 14 hrs post-treatment.

FIG. 75A-B shows an exemplary Bifidobacterial modulation of cytokine responses between controls (no bacteria); Bif1 vs. Bif2; with or without TNF-α/IL-1β. FIG. 75A TNF-α cytokine production at 4 hrs post-treatment. FIG. 75B TNF-α cytokine production at 14 hrs post-treatment.

FIG. 76A-B shows an exemplary Bifidobacterial modulation of cytokine responses between controls (no bacteria); Bif1 vs. Bif2; with or without TNF-α/IL-1β. FIG. 76A IL-1β cytokine production at 4 hrs post-treatment. FIG. 76B IL-1β cytokine production at 14 hrs post-treatment.

FIG. 77A-B shows an exemplary Bifidobacterial modulation of cytokine responses between controls (no bacteria); Bif1 vs. Bif2; with or without TNF-α/IL-1β. FIG. 77A IFN-gamma cytokine production at 4 hrs post-treatment. FIG. 77B IFN-gamma cytokine production at 14 hrs post-treatment.

FIG. 78A-B shows an exemplary Bifidobacterial modulation of cytokine responses between controls (no bacteria); Bif1 vs. Bif2; with or without TNF-α/IL-1β. FIG. 78A IFN-gamma cytokine production at 4 hrs post-treatment. FIG. 78B IL-12p70 cytokine production at 14 hrs post-treatment.

FIG. 79A-C shows an exemplary description of the establishment of the Colon-Chip FIG. 79A Representation of the timeframe of the protocol, FIG. 79B) Schematic of the different compartments of the chip as well as the location of the specific cell types, FIG. 79C shows an exemplary representative brightfield contrast phase images of the colonic epithelial and endothelial cells before and at the timepoint of seeding.

FIG. 80A-C shows an exemplary assessment of Mucin2 (Muc2) expression across three different healthy donors in a LLI culture FIG. 80A Representative image of immunofluorescence against Muc2 at the day 8 of fluidic culture, FIG. 80B Quantification of immunofluorescence for Muc2 expression, FIG. 80C q-PCR for the expression of Muc2. Mean±SD, n=3-10 chips/donor

FIG. 81A-C shows an exemplary comparison of Mucin2 (Muc2) expression between a LLI and a Periodical ALI culture of Donor 1 derived colonocytes FIG. 81A Representative image of immunofluorescence against Muc2 at the day 8 of fluidic culture, FIG. 81B Quantification of immunofluorescence for Muc2 expression, FIG. 81C q-PCR for the expression of Muc2. Mean±SD, n=3 chips/condition, t-test, **: p<0.01.

### DEFINITIONS

“Intestinal motility” refers to intestinal function including but not limited to ciliary movements, villi movements, smooth muscle contractions, etc.

Intestinal motility disorders in general refers to abnormal intestinal movements, including but not limited to contractions, such as spasms or intestinal paralysis, such as experienced in Irritable bowel syndrome (IBS); fecal incontinence; constipation, etc. Such disorders may manifest in a variety of ways, including but not limited to the following: abdominal distention (bloating); recurrent obstruction; severe constipation, etc. In other words, an intestinal motility disorder may refer to any alteration in the transit of foods and/or secretions into the digestive tract, such as where movement of intestinal contents is too slow or so disrupted that it causes the contents to become backed up as if there is a blockage. Alternatively, an intestinal motility disorder may result from where movement of intestinal contents through the intestine is too fast resulting in diarrhea and long term resulting in malnutrition.

“Probiotic” in general refers to live microorganisms that confer a health benefit on the host when administered in adequate amounts. Such organisms are contemplated to survive both gastric acid and bile to reach at least the small intestine and some as far as the colon, where they exert their effects. In particular, as used herein, a product with an adequate number of microorganisms at time of consumption specifically shown to confer health benefits in controlled human trials is considered a “therapeutic probiotic”.

“Prebiotic” refers to dietary substances that nurture specific changes in the composition and/or activity of the gastrointestinal microbiota (favoring beneficial bacteria), thus conferring benefit(s) upon host health.

“Synbiotics” refer to products that contain both probiotics and prebiotics.

The term “microfluidic” as used herein relates to components where moving fluid is constrained in or directed through one or more channels wherein one or more dimensions are 1 mm or smaller (microscale). Microfluidic channels may be larger than microscale in one or more directions, though the channel(s) will be on the microscale in at least one direction. In some instances the geometry of a microfluidic channel may be configured to control the fluid flow rate through the channel (e.g. increase channel height to reduce shear). Microfluidic channels can be formed of various geometries to facilitate a wide range of flow rates through the channels.

“Channels” are pathways (whether straight, curved, single, multiple, in a network, etc.) through a medium (e.g., silicon) that allow for movement of liquids and gasses. Channels thus can connect other components, i.e., keep components “in communication” and more particularly, “in fluidic communication” and still more particularly, “in liquid communication.” Such components include, but are not limited to, liquid-intake ports and gas vents. Microchannels are channels with dimensions less than 1 millimeter and greater than 1 micron.

As used herein, the phrases “connected to,” “coupled to,” “in contact with” and “in communication with” refer to any form of interaction between two or more entities, including mechanical, electrical, magnetic, electromagnetic, fluidic, and thermal interaction. For example, in one embodiment, channels in a microfluidic device are in fluidic communication with cells and (optionally) a fluid reservoir. Two components may be coupled to each other even though they are not in direct contact with each other. For example, two components may be coupled to each other through an intermediate component (e.g. tubing or other conduit).

As used herein, the term “biopsy” refers to a sample of the tissue that is removed from a body.

As used herein, “Caco-2” or “Caco2” refer to a human epithlial intestinal cell line demonstrating a well-differentiated brush border on the apical surface with tight junctions between cells. Although this cell line was originally derived from a large intestine (colon) carcinoma, also called an epithelial colorectal adenocarcinoma, this cell line can express typical small-intestinal microvillus hydrolases and nutrient transporters, see. Meunier, et al., “The human intestinal epithelial cell line Caco-2; pharmacological and pharmacokinetic applications.” Cell Biol Toxicol. 11(3-4):187-94, 1995, abstract. Examples of Caco-2 cell lines include but are not limited to CRL-2102, American Type Culture Collection (Rockville, Md.); a BBE subclone of Caco-2 cells; etc.

“Physiological hypoxia” refers to an oxygen level at which tissues respond to maintain their preferred oxygen level. This can be by physiological means, e.g. vasodilation, increasing blood flow, and/or upregulation of hypoxia response genes. “Physiological hypoxia” may occur at approximately 1% oxygen, with an upper limit <5%, or ranging from 0.1-2%, but may be triggered at different levels, such as higher levels for lung tissue. Thus, oxygen levels for triggering “Physiological hypoxia” depend upon the type of tissue or cells.

“Normoxia” or “Atmospheric Oxygen” refers to oxygen levels approximately 20-21% O₂ while “Normoxic” conditions refer to CO₂ maintained around 5%. Oxygen levels universally measured in vivo in normal nonlung tissues averages about 5% oxygen, typically ranging from about 3% to 7.5%. Oxygen levels are around 14.5% in normal lung tissue. Thus, “physiological normoxia” or “physoxia” refers to oxygen levels between 3-7.5%, in nonlung tissue, as representing physiological levels of oxygen (O₂) in tissues of approximately 5%.

As used herein, 21% O₂ incubation conditions as normal microenvironments for cell incubation are compared to 5% O₂ levels referred to as physoxia microenvironments.

For reference, “Oxygen-free” in general refers to an anaerobic environment, however the use of the term “anaerobic” is not limited to oxygen-free. For one example, an “anaerobic microbe” may be an “obligate anaerobe” which may fail to thrive or die in the presence of O₂ or a “facultative anaerobe” which may thrive in both an anaerobic an aerobic environment. However there may be exceptions. As one example, Clostridia spp. typically refers to obligate anaerobic and nutritionally fastidious bacteria However as described herein, Clostridia symbiosum are shown to be present when co-cultured on chip under physoxia microenvironments.

### DESCRIPTION OF THE INVENTION

The present invention relates to a combination of microbes, cell culture systems and microfluidic fluidic systems. In one embodiment, these are used to provide a Lung On-Chip co-culture with microbes. In one embodiment, these are used to provide a human Intestine On-Chip with optimal intestinal motility. More specifically, in some embodiments, a microfluidic chip containing intestinal epithelial cells co-cultured with intestinal endothelial cells in the presence of bacteria, such as probiotic bacteria, may find use in providing an Intestine-On-Chip for testing intestinal motility function and/or for simulating host-biome interactions. In some embodiments, an Intestine On-Chip may be used for identifying (testing) therapeutic compounds continuing probiotic microbes or compounds for inducing intestinal motility for use in treating gastrointestinal disorders or diseases related to intestinal function.

Many publications indicate that enteric microbiota populations contribute to gastrointestinal health. In fact, intestinal bacteria play a role in maintaining immune and metabolic homeostasis and protecting against pathogens. Thus, healthy people consume many types of probiotic products in attempts to manipulate their intestinal microbiota for obtaining a health benefit. Because publications further describe that altered gut bacterial composition (dysbiosis) is associated with the pathogenesis of many inflammatory diseases and infections, patients consume probiotic products in attempts to manipulate their intestinal microbiota to obtain relief from an intestinal disorder and/or relief from disease symptoms, including intestinal autoimmune conditions and intestinal cancer. Further, altered compositions of intestinal microbiota are now associated with obesity in some people. Therefore, there is a need for testing and identifying bacteria strains that may confer a health benefit, alone or in combination, for any one or more of these conditions when administered in a manner to reach target areas of the gastrointestinal system.

One example of a product considered to confer a probiotic health effect on the intestines include, but are not limited to: L. rhamnosus GG (LGG) in Culturelle (Valio, Helsinki, Finland/Amerifit Brands, Inc., Cromwell, Conn.) and Danimals yogurt, The Dannon Company, Inc. PO Box 1625 Horsham, Pa. 19044). Its effectiveness was rated for certain types of disorders varying anywhere from a strong positive effect shown by well-conducted controlled studies in the primary literature; to some positive effect shown by controlled studies but with the presence of some negative studies or inadequate amount of work to establish the certainty it would provide a benefit; to some positive studies but clearly inadequate amount of work to establish the certainty it would provide a benefit. See, Ciorba, “A Gastroenterologist's Guide to Probiotics.” Clin Gastroenterol Hepatol. September; 10(9): 960-968.2012.

It is believed that many probiotic strains do not actually colonize the gut because they are no longer recoverable in stool 1-4 weeks after stopping consumption. Further, in some studies, the probiotic product described did not change the gut's overall bacterial composition, but instead altered gene expression patterns relevant to carbohydrate metabolism in the host's resident gut microbes. These changes in human fecal “metatranscriptome” were transient, confined merely to the time of the probiotic consumption. Thus, if a sustained benefit from a probiotic is desired, continued consumption is likely required.

Another examples of a product considered to confer a probiotic effect includes, but is not limited to: Bifidobacterium infantis 35624 in Align (Proctor & Gamble, Cincinnati, Ohio), suggested fro treating IBD. However, this strain was rated as having some positive effect in controlled studies but the presence of some negative studies or inadequate amount of work was done to establish the certainty it would provide a benefit to patients with IBD. Thus it comes with a caution for expecting any relief from symptoms. See, Ciorba, “A Gastroenterologist's Guide to Probiotics.” Clin Gastroenterol Hepatol. September; 10(9): 960-968.2012.

Therefore, a more effective means of determining whether a potential probiotic strain would provide a benefit to a patient is needed. Even further, a more effective means of determining whether a potential probiotic strain would provide a specific type of benefit to intestinal function is needed.

Current systems fail to recapitulate in vivo biology and do not accurately predict human response to disease, medicines, chemicals, foods, etc. Organs-on-Chips, such as those described herein, recreate “true-to-life” human biology in vitro. Thus organs-on-chips enable better predictions of human response and new understandings of human biology in vitro. In some embodiments microfluidic organ chips comprise small molecules and biologics. In some embodiments microfluidic organ chips from different organs and areas of the human body are linked together.

Intestinal organoids, are obtained from biopsies, contain relevant cell types for use in microfluidic chips. Thus, in some embodiments, an Intestine-Chip provides the microenvironment in which these organoids maintain their 3D structure and immune cell function.

Lactobacillus spp. typically refer to facultative anaerobic and nutritionally fastidious bacteria Clostridia spp. typically refers to an obligate anaerobic and nutritionally fastidious bacteria However as described herein, Clostridia symbiosum are shown to be present when co-cultured on chip under physoxia microenvironments.

### I. Intestinal Homeostasis: Intestine (Organ) On-Chip as a Clinical Model of Intestinal Motility and Health.

Intestinal homeostasis refers to interactions between microenvironments comprising microbial cells; parenchymal cells; parenchymal tissues; extracellular sections; lamina propria; stroma and stromal cells; fluids (extracellular fluids such as luminal fluid, interstitial fluid (between cells), plasma, etc.; and intracellular fluid), etc. More simply, intestinal homeostasis may also refer to interactions between luminal microbes, host epithelium, and resident immune cells of the lamina propria. Together, the signals from these tissues are integrated to define the balance between healthy and diseased states. The development of new therapeutic interventions that safely and effectively manipulate these signals to promote or restore the health state requires experimental models that (1) more accurately predict human responses and (2) enable experimental control to understand how signals are processed at the organ-level. Animal models often fail to accurately predict human responses to drugs while current in vitro systems cannot capture the complexity of the whole organ context. The human Intestine-Chip, described herein, places living cells in micro-engineered environments within fluidic devices to provide a platform for more control of experimental variables and for providing an in vitro simulation of the intestinal milieu.

Exemplary components contributing to intestinal physiology and intestinal homeostasis: including but not limited to Environment, e.g. Microbiome, Diet, etc.; Epithelial Barrier, e.g. Genetic susceptibility to IBD, etc.; Mucosal Immunity, e.g. Sensitivity to commensal antigens, etc.; Enteric Nervous System, e.g. Motility, etc. Thus additional unmet needs include but are not limited to: Intestinal models that recapitulate factors that drive pathogenesis of disease (e.g. Colorectal Cancer). Inflammatory Bowel Diseases (IBD); mimic risk factors or environments, e.g. Crohn's Disease (CD), ulcerative colitis (UC), etc., and factors where regional differences in diseases or disorders are profound, i.e. greatly different.

Intestine On-Chip Models of Disease contemplated for use herein, individually or combined, with microbial co-cultures include but are not limited to: providing Tissue-level Cytoarchitectures, e.g. Epithelial-immune interactions, Recruitment of peripheral immune cells, etc.; Recapitulating Physiological Microenvironments, e.g. Mechanoactuation (Peristalsis, Stretch, Motility), e.g. Physiological extracellular matrix, etc.; Patient-Specific Phenotypes, e.g. Host genetic factors (Epithelial, immune responses), e.g. Epigenetic factors; in addition to Microbial compositions; Drug responses, etc.; for use in clinical applications such as disease population matched patients, genetically population matched patients, individual patients, etc. In other words, Intestine On-Chip Models of Disease provide A platform to identify factors that weaken the epithelial barrier, investigate disease mechanisms and associated biomarkers, allow better understanding of responses to drugs in a patient-specific, or patient population specific fashion.

In one embodiment, intestinal homeostasis may be interactions between discrete components, including but not limited to: luminal microbes, host epithelium, and resident immune cells of the lamina propria.

Signals (such as changes in gene expression, cytokine secretion, mucus secretion, protein expression and/or function, permeability changes, barrier changes, pH changes, etc.) from microenvironments in intestinal tissues integrate in vivo as a macro environment to define the balance between healthy and diseased states.

Thus, development of new therapeutic interventions for safely and effectively manipulating these signals to promote or restore a more healthy state (homeostasis) requires experimental models in vitro that (1) more accurately predict human responses to a particular signal manipulation or therapeutic intervention and (2) enable experimental control of variables in order to understand how signals are processed at the organ-level, i.e. using engineered organ mimics on microfluidic chips that are beyond the complexity of mere transwell co-cultures.

Moreover, animal models fail to accurately predict human responses to drugs (therapeutics) while current in vitro systems fail to capture the complexity of the whole organ context.

However, microfluidic human Intestine On-Chip has living cells seeded into channels creating micro-engineered environments to provide experimental control of variables and recapitulating aspects of the intestinal milieu associated with responding to therapeutics.

### II. Identifying Probiotic Bacterial Strains for Guided (Including Personal) Therapy for Maintaining and Restoring Intestinal Function.

In some embodiments, an engineered microfluidic Intestine On-Chip may provide a platform for associating epithelial and endothelial responses to inflammation as a robust bioassay capable of identifying probiotic bacterial strains.

In some embodiments, uses of such identified probiotic bacterial strains include but are not limited to: selection of therapeutic probiotic strains for certain types of disorders or diseases; selection of bacteria strains for use as probiotic starters for the improvement of the traditional fermentation processes, such as yogurt, olives, etc., and the production of new types of functional foods; selection of bacteria strains to be used as starter culture in plant-based fermented foods.

In some embodiments, identifying a therapeutic probiotic bacteria strain is based upon distinguishing a candidate bacteria strain added to a microfluidic intestine on-chip over a comparison bacteria strain added to a duplicate microfluidic intestine on-chip. Thus, when a candidate bacteria strain induces an improved characteristic within 2 to 3 days of incubation in the microfluidic intestinal chip, over the comparison strain, the candidate probiotic strain is considered a therapeutic probiotic strain. Thus, a candidate therapeutic probiotic bacterial strain may have one or more properties including but not limited to: increased (improved) barrier function, increased expression of tight junction proteins such as ZO-1, increased mucin production (e.g. increased MUC2 expression), inducing more pronounced villi-like invaginations, altering intestinal motility, i.e. increasing motility for certain conditions, decreasing motility for other conditions, etc.

Further, screening candidate bacteria strains on embodiments of a microfluidic intestine on-chip, provides a means for establishing probiotic potential, e.g., as a potential therapeutic probiotic for reducing inflammation, including but not limited to vascular inflammation. ICAM-1 expression is one readout of vascular inflammation and one predictor of immune cell recruitment.

Additionally, prophylactive effects are indicative of a therapeutic probiotic bacterial strain as identified in one embodiment of a microfluidic intestine on-chip. prophylactive effects include but are not limited to inducing an improved characteristic within 2 to 3 days of incubation in one embodiment of a microfluidic intestine on-chip over a comparison strain tested in a duplicate embodiment of a microfluidic intestine on-chip. As one example, in some embodiments, a significant decrease in IL-6 and IL-10 production in response to TNF-α/IL-1β induction indicates a prophylactive effect upon an intestine on-chip.

In fact, in a blinded study, we co-cultured bacteria in the Intestine-Chip for multiple days and observed the modulation of host epithelial and endothelial responses after inflammatory stimulation. We were then able to distinguish between clinically relevant probiotic strains and a non-probiotic control strain. Thus, these methods are contemplated for identifying a probiotic strain of bacteria.

Inflammatory treatment with TNF-α and IL-1β result in disrupted barrier function, increases production of IL-8 and IL-6, and unregulates endothelial addressin ICAM-1.

Co-culture with lactobacilli strain 1 or bifdobacteria strain 2 impacted these intestinal and immune specific readouts and allowed for discrimination between strains with likely probiotic function.

Conclusions: Organ-Chip technology provides unique windows into human organ level function that allow for precise control of tissue microenvironments. Here we demonstrate the potential of the Intestine-Chip platform to identify and assess the probiotic function of commensal microbial strains.

Contemplated experiments will focus on further development of the Intestinal mucosa investigating the microbial-immune axis.

Examples of bacteria strains used here in intestine on-chips are Lactobacillus rhamnosus strain GG and Bifidobacterium spp. While both Bifidobacteriumand Lactobacilli bacteria share some metabolic properties, Lactobacilli have a much higher level of phylogenetic, phenotypic, and ecological diversity and have over 170 recognized species.

Organ-Chip technology provides unique windows into human organ level function that allow for precise control of tissue microenvironments. In some embodiments, the potential of the Intestine-Chip platform is contemplated to identify and assess the probiotic function of commensal microbial strains. Contemplated experiments will focus on development of the Intestinal mucosa investigating the microbial-immune axis.

### A. Exemplary Probiotic Bacteria.

Exemplary candidate probiotic bacteria strains were developed as altered strains of known probiotic stains used for comparison.

### 1. Lactobacillus spp.

Lactobacillus, (genus Lactobacillus), refers to any of a group of rod shaped, gram-positive, non-spore-forming bacteria of the family Lactobacillaceae. Various species of Lactobacillus are used commercially during the production of sour milks, cheeses, and yogurt, and they have a role in the manufacture of fermented vegetables (pickles and sauerkraut), beverages (wine and juices), sourdough breads, and some sausages. Lactobacillus are generally nonmotile and can survive in both aerobic and anaerobic environments. Lactobacillus species include but are not limited to: L. acidophilus, L. brevis, L. casei, and L. sanfranciscensis. Commercial preparations of lactobacilli are used as probiotics to restore normal flora after the imbalance created by antibiotic therapy.

As one example, a probiotic strain of Lactobacillus rhamnosus strain GG (Gorbach and Goldin): (LGG) (Lactobacillus acidophilus (Moro) Hansen and Mocquot: Lactobacillus rhamnosus (Hansen) Collins et al., ATCC 53103) is a Gram-positive bacteria that was isolated from the human gastrointestinal tract.

Features include but are not limited to: extensive published human clinical data for reference; digestible, etc. Thus, LGG1 was compared to a non-probiotic strain LGG2.

As shown herein, LGG1 provides mucosal adherence; immunomdulatory and improvement of barrier function on-chip.

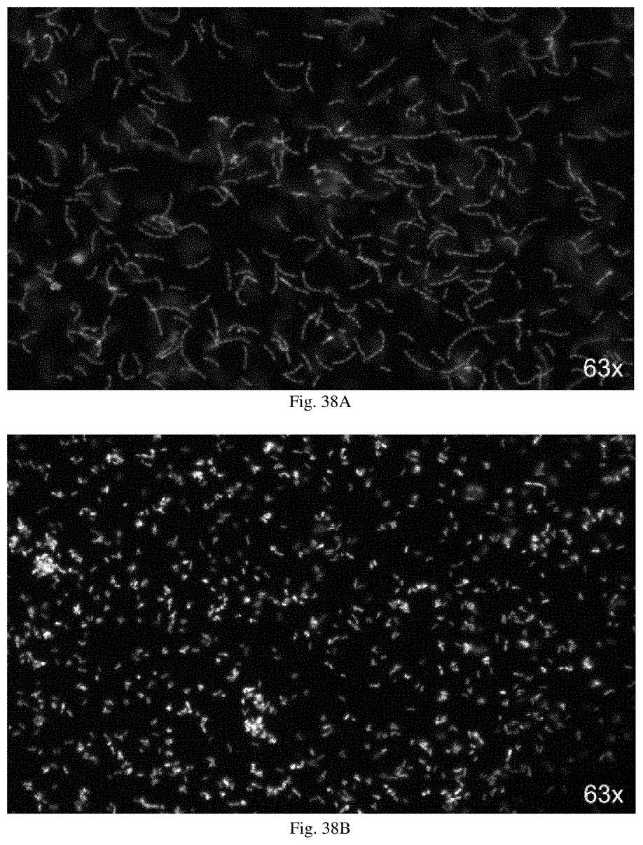

FIG. 38A Micrograph of Lactobacilli bacteria.

### 2. Bifidobacterium spp.

As another example, a probiotic strain of Bifidobacteria, a type of lactic acid bacteria, was compared to a nonprobiotic strain. Bifidobacteria (B.) species in general refer to an anaerobic lactic acid bacteria that naturally inhabits our intestinal and urogenital tracts, and they are some of the first bacteria to colonize our bodies when we are born. In fact, Bifidobacteria species are believed to make up the vast majority (over 90%) of all bacteria in the guts of breastfed infants. In adults, one strain, B. longum is reported to promote good digestion, boosts the immune system, and produced lactic and acetic acid, acids that contribute to controlling intestinal pH. B. longum is also are reported to inhibit the growth of Candida albicans (fungi), E. coli, and pathogenic bacteria. Commercially available strains include but are not limited to: B. longum bb536; B. longum es 1; B. longum w11; B. longum NCC 3001; B. longum 1714; B. longum KACC 91563; B. longum spp. longum SPM 1205, 1206, 1207, CECT 7347, MM-2; B. longum subsp. infantis ATCC 15697.

As an exemplary bacteria strain used herein, Bifidobacteria has extensive published human clinical data for reference; thrives in an anaerobic microenvironment; has a unique carbohydrate metabolism; and produces fermentation product(s) linked to probiotic functions. However, it was surprisingly found that this organism would also grow in an aerobic microenvironment on one embodiment of a microfluidic intestine on-chip

Bifidobacteria spp., including but not limited to several strains of Bif. FIG. 38B Micrograph of Bifidobacteria, a type of lactic acid bacteria, is a rod-shaped bacterium frequently observed with branches, e.g. Y shaped.

B. Distinguishing Probiotic Bacteria Strains Over Nonprobiotic Strains.

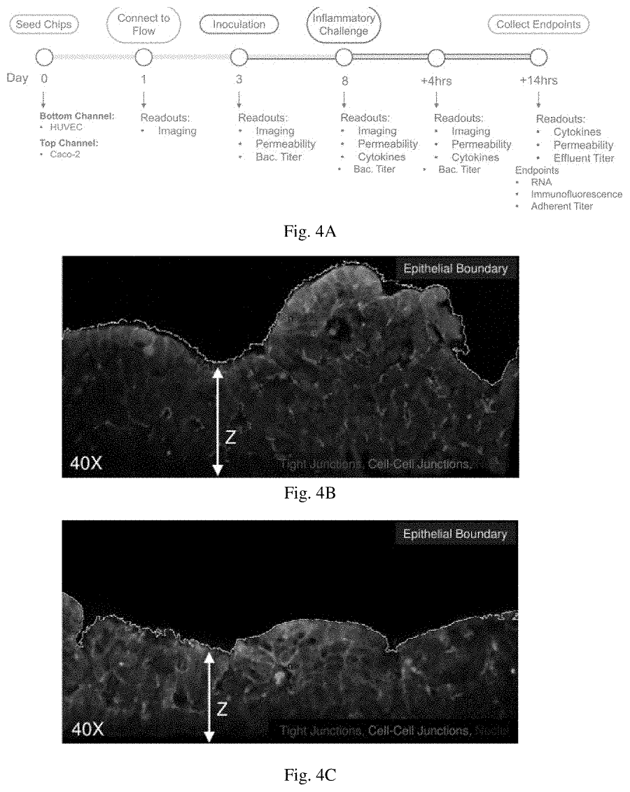

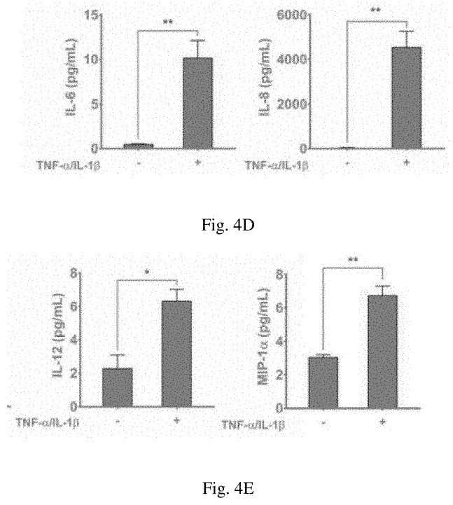

In some embodiments, a contemplated experimental plan is designed. In one embodiment of an intestine on-chip, on Day 0, the bottom channel is seeded with HUVECs, while the top channel is seeded with Caco-2 cells. See additional timeline details in FIG. 4 A-E.

FIG. 4 A-E shows an Exemplary Contemplated Experimental Plan and Timeline.
Day 0: Seed Chips: Bottom Channel: HUVEC. Top Channel: Caco-2. Day 1: Connect to flow. Day 3: Inoculation and Readouts: Imaging; Permeability; Bacterium Titer; etc. Day 8: Inflammatory Challenge. Readouts: Imaging; Permeability; Cytokines; Bacterium Titer; etc. Day 8 +4 hrs: Readouts: Imaging; Permeability; Cytokines; Bacterium Titer; etc. Day 8 +14 hrs: Readouts: Permeability; Cytokines; Effluent Titer; etc. Day 8 +14 hrs: Collect Endpoints: RNA; Immunofluorescence; Adherent Titer; etc.

In particular, bacterial effects are measured by readouts including microbial modulation of permeability and intestinal motility. Readouts further include but are not limited to: a secreted cytokine profile: examples, IL6, IL8, IL10, IL12, TNF-α, IL-1β, INF-γ, etc.; small molecule permeability, example, using Inulin-FITC; Immunofluorescence of intestinal cell markers, cell viability, etc.; including but not limited to cell-cell junctions: example, staining for E-cadherin, ZO-1, etc.; Epithelial differentiation, example, MUC2 expression, etc.; Endothelial selectin expression: ICAM1, etc.; Gene expression; Epithelial barrier function: ZO-1, Claudins 1, Occludin, etc.; Endothelial selectin expression, etc. Moreover, Screening capability: speed and throughput, i.e. numbers of chips evaluated over time, and time to completion of readout assays will be evaluated.

The following Table 1 shows one exemplary embodiment of comparative testing conditions for identifying a probiotic strain in comparison to a nonprobiotic strain of bacteria.

TABLE 1
Exemplary Testing Conditions for identifying a probiotic
strain over a nonprobiotic strain of bacteria.

| Strain | Treatment | |
|---|---|---|
| — | 1. | Control |
| | 2. | +TNFα/IL-1β (30 ng/mL) |
| — | 1. | Control |
| | 2. | +Strain1 |
| +Strain 1 | 1. | Control |
| | 2. | +TNFα/IL-1β (30 ng/mL) |
| — | 1. | Control |
| | 2. | +Strain2 |
| +Strain 2 | 1. | Control |
| | 2. | +TNFα/IL-1β (30 ng/mL) |

Bacterial effects are measured by readouts including microbial modulation of permeability and intestinal motility. Readouts further include but are not limited to: a secreted cytokine profile: examples, IL-6, IL-8, IL1-0, IL-12, TNF-α, IL-1β, INF-γ, etc.; Small molecule permeability, example, using Inulin-FITC; Immunofluorescence of intestinal cell markers, cell viability, etc.; including but not limited to cell-cell junctions: example, staining for E-cadherin, ZO-1, etc.; Epithelial differentiation, example, MUC2 expression, etc.; Endothelial selectin expression: ICAM1, etc.; Gene expression; Epithelial barrier function: ZO-1, Claudins 1, Occludin, etc.; Endothelial selectin expression, etc. Moreover, Screening capability: speed and throughput, i.e. numbers of chips evaluated over time, and time to completion of readout assays will be evaluated.

### 1. Lactobacillus rhamnosus strain GG (LGG) Bacteria Strains:

### Comparative Results Between Two Strains.

Lactobacillus spp. (including but not limited to several strains of LGG) Features include but are not limited to: Extensive published human clinical data for reference; digestible; shown herein to provide mucosal adherence; immunomdulatory and improve barrier function on-chip.

Exemplary results of TNF-α/IL-1β treatment alone shows that such treatment does not significantly affect the Intestine-Chip morphology in one embodiment of an Intestine On-Chip.

FIGS. 38A-B shows exemplary Epithelial Morphology in representative brightfield images of Intestine On-Chip. FIG. 38A Control Chip (no bacteria added), Day 8. FIG. 38B Chip treated with +TNF-α/IL-1β, Day 8 (no bacteria added).

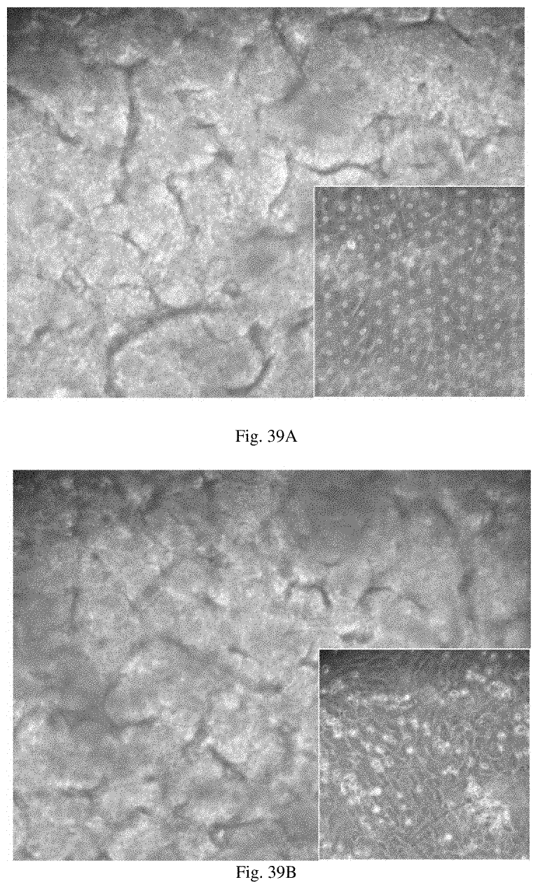

FIGS. 39A-B shows exemplary Epithelial Morphology in representative bright-field images of Intestine On-Chip, untreated. FIG. 39A Control Chip (no bacteria added), Day 8. FIG. 39B Chip treated with LGG1, Day 8.

LGG1 alone has an effect on epithelial tissue morphology under nonstimulatory conditions in one embodiment of an Intestine On-Chip.

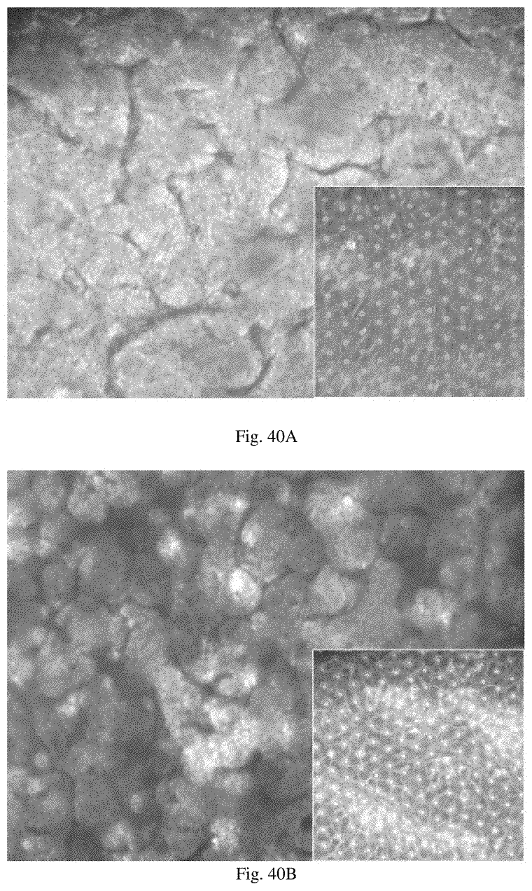

FIGS. 40A-B shows exemplary Epithelial Morphology in representative bright-field images of Intestine On-Chip, untreated. FIG. 40A Control Chip (no bacteria added), Day 8. FIG. 40B Chip with LGG1, Day 8.

Additionally, LGG1 effects epithelial tissue morphology under stimulatory conditions in one embodiment of an Intestine On-Chip.

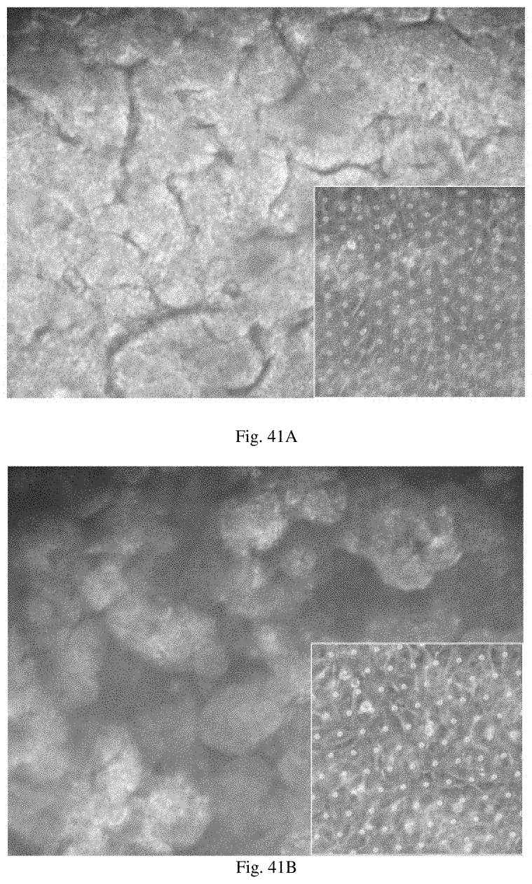

FIGS. 41A-B shows exemplary Epithelial Morphology in representative bright-field images of Intestine On-Chip, treated. FIG. 41A Control Chip (no bacteria added), Day 8. FIG. 41B Chip treated with +LGG1 in the presence of +TNF-α/IL-1β, Day 8.

In contrast to LGG1, LGG2 alone showed no significant epithelial morphology changes under nonstimulatory conditions in one embodiment of an Intestine On-Chip.

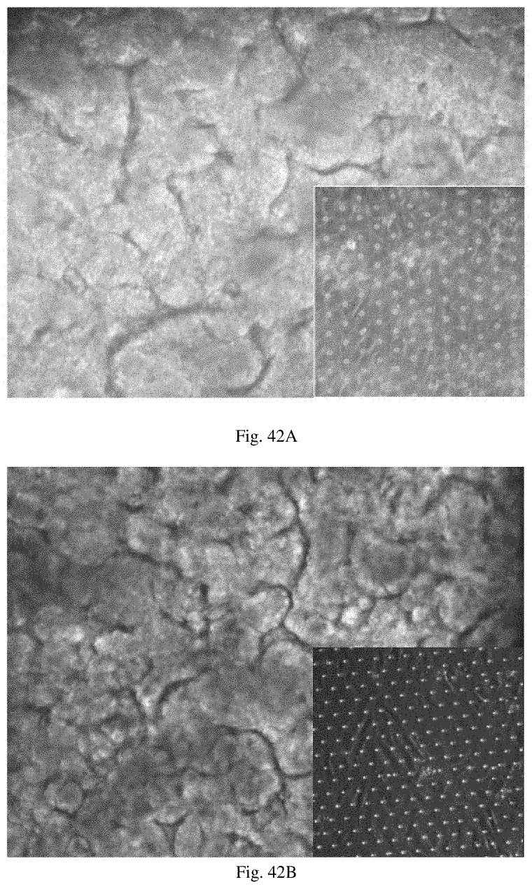

FIGS. 42A-B shows exemplary Epithelial Morphology in representative bright-field images of Intestine On-Chip, untreated. FIG. 42A Control Chip (no bacteria added), Day 8. FIG. 42B Chip treated with +LGG2, Day 8.

Further, LGG2 showed that no significant epithelial morphology change was observed under stimulatory conditions in one embodiment of an Intestine On-Chip.

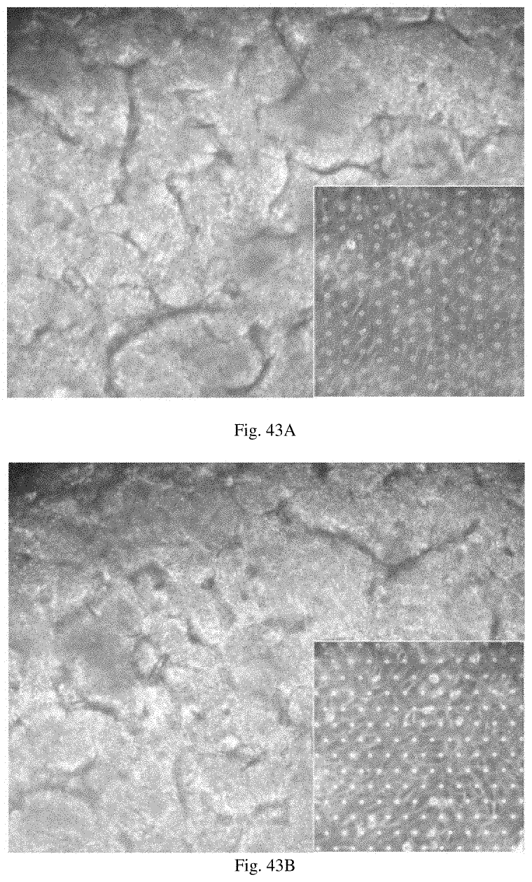

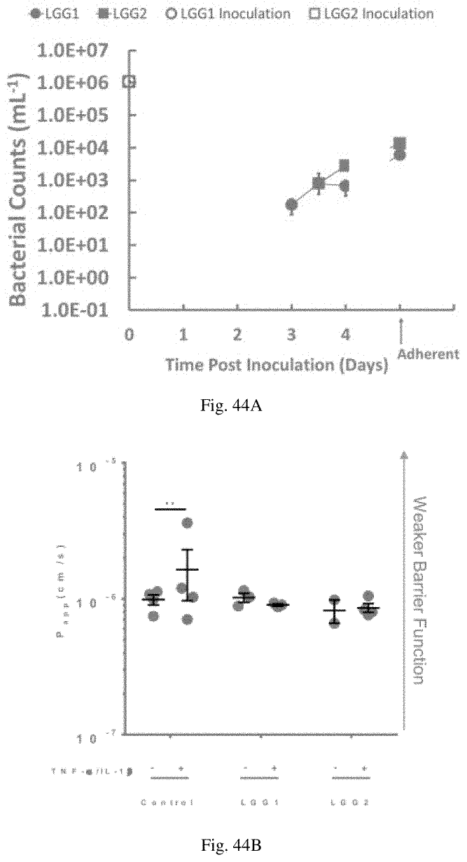

FIGS. 43A-B shows exemplary Epithelial Morphology in representative bright-field images of Intestine On-Chip, treated. FIG. 43A Control Chip (no bacteria added), Day 8. FIG. 43B Chip treated with +LGG2 in the presence of +INF-α/IL-1β, Day 8.

2. LGG Barrier Function.

Barrier function is an indicator of epithelial cell health in the intestine. As shown herein, both Lactobacilli strains (LGG1 and LGG2) colonize one embodiment of an Intestine On-Chip without disrupting epithelial barrier function. Each bacteria strain was added (inoculated) on chip at a concentration of 1×10⁶ mL⁻¹.

No significant difference was observed in permeability between control (no bacteria) and either LGG strains suggesting a healthy barrier is maintained on-chip in the presence of each of these LGG strains. Furthermore, the barrier generally is not disrupted by INF-α/IL-1β treatment as used herein.

FIGS. 44A-B shows exemplary Lactobacilli Strains that Stably Colonized the Intestine-Chip in part shown by exemplary read-outs of bacterial titration and apparent permeability. FIG. 44A Bacterial Titration (counts mL⁻¹) at Day 3 and Day 4 compared to adherent counts at Day 4. FIG. 44B Apparent Permeability demonstrating improved barrier function after either Lactobacilli LGG1 and LGG2 infection.

3. LGG Secreted Cytokines.

These Lactobacilli strains tested significantly affect the observed cytokine response to inflammatory challenge. Thus, both Lactobacilli strains significantly altered the observed cytokine response to inflammatory challenge. However, the kinetics of the cytokine response are differentially regulated by each LGG strain.

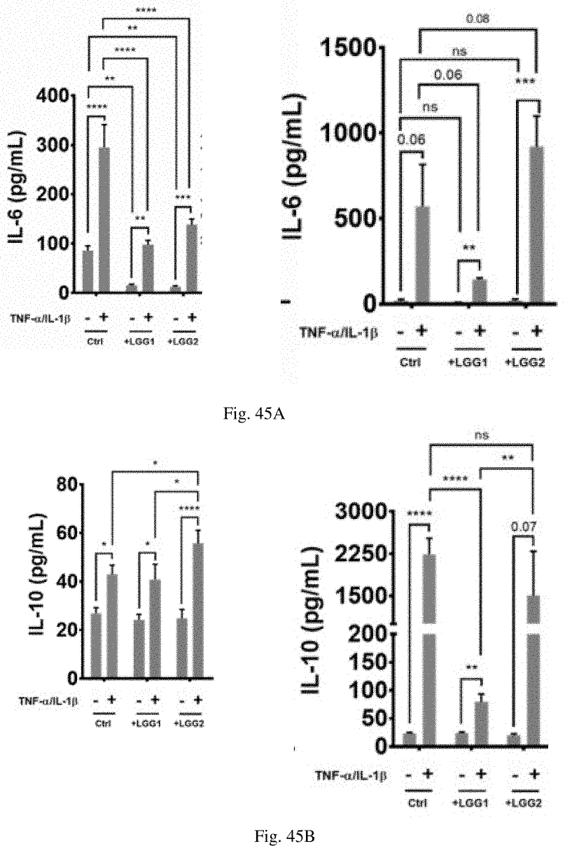

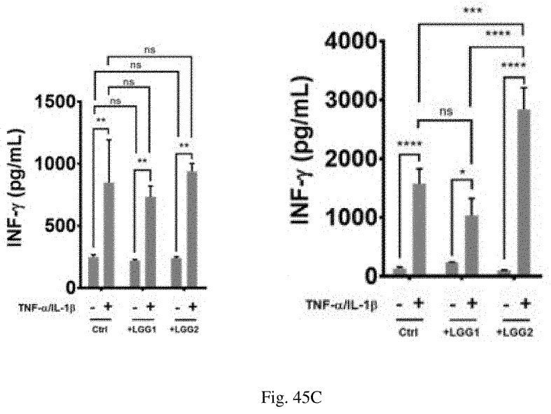

After 4 hours post cytokine stimulation, both LGG1 and LGG2 suppressed IL-6 secretion compared to cytokine stimulation alone. LGG2 induced IL-10 secretion unlike LGG1 which did not appear to alter IL-10 secretion compared to cytokine stimulation alone. Neither strain significantly alters IFN-gamma section. FIGS. 45A-C, left graphs.

After 14 hours post cytokine stimulation, LGG2 induced higher levels of IL-6, IL-10 and IFN-gamma than LGG1. However, while LGG2 induced higher levels of IL-6 and IFN-gamma than cytokine stimulation alone, IL-10 was about the same. In contrast, LGG1 significantly lowered IL-6, IL-10 and about the same amount of secreted IFN-gamma compared to cytokine stimulation alone. Therefore, LGG1 demonstrates anti-inflammatory characteristics over time in relation to IL-6 and IL-10, while in contrast to LGG2 not significantly increasing IFN-gamma, when added to one embodiment of an intestine on-chip stimulated with IFN-a/IL-B. FIGS. 45A-C, right graphs.

Significant differences between LGG1 and LGG2 effects are demonstrated when comparing IL-6 secretion between 4 and 14 hours post stimulation with IFN-a/IL-B. While LGG1 (blue box and lines) suppresses IL-6 secretion in the presence of IFN-a/IL-B compared to IFN-a/IL-B stimulation alone (upper grey box and lines), see FIG. 45A. In contrast, LGG2 (blue box and lines) shows similar secretion amounts in the presence of IFN-a/IL-B compared to IFN-a/IL-B stimulation alone (upper grey box and lines), see FIG. 45B.

FIGS. 45A-C shows exemplary Lactobacilli Strains (LGG1 And LGG2) Modulate Epithelial Cytokine Responses. Duplicate chips were cultured with or without stimulatory TNF-α/IL-1β, then with or without one of the LGG test strains. FIGS. 45A IL-6 (pg/ml) Time Post Treatment 4 hr (left) 14 hr (right). FIGS. 45B IL-10 (pg/ml) Time Post Treatment 4 hr (left) 14 hr (right). FIGS. 45C IFN-gamma (pg/ml) Time Post Treatment 4 hr (left) 14 hr (right).

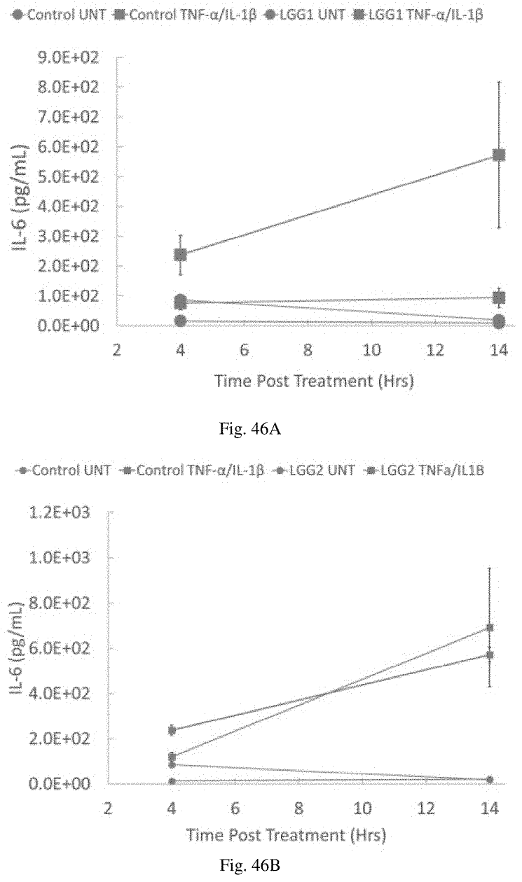

FIGS. 46A-B shows exemplary Lactobacillus modulation of the IL-6 Epithelial Cytokine Response. FIGS. 46A shows IL-6 Cytokine Production +LGG1. FIGS. 46B shows IL-6 Cytokine Production +LGG2.

4. LGG Selectin Expression.

LGGs upregulate endothelial adhesion molecules, including selectin expression, and significantly modulate addressin expression in response to inflammatory challenge as measured by qPCR.

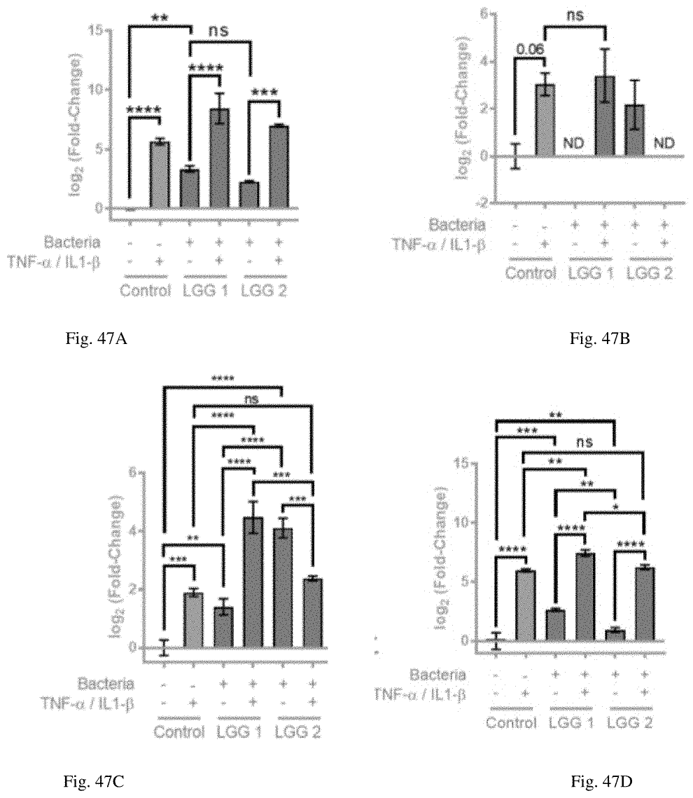

LGG1 and LGG2 increase ICAM (Intercellular Adhesion Molecule) (mucosal vascular addressin cell adhesion molecule/endothelial cell adhesion molecule) expression compared to unstimulated controls. Under cytokine stimulation, both LGG1 and LGG2 and TNFa-/IL1-B alone increase ICAM expression. FIG. 47A ICAM.

LGG effects on MADCAM (mucosal vascular addressin cell adhesion molecule) were not completely demonstrated, however there was little difference measured between the increased MADCAM expression with or without LGG1 under stimulatory conditions. LGG2 was show to induce MADCAM expression without stimulation with cytokines. FIG. 47B.

LGG1 upregulates PECAM (Platelet endothelial cell adhesion molecule) more than LGG2 under conditions of cytokine stimulation. In fact, LGG2 increases PECAM without stimulation then reduces expression after stimulation. As opposed to LGGG1 that also insreases PECAM without stimulation but unlike LGG2, LGG1 significantly increases PECAM under cytokine stimulation conditions. FIG. 47C.

LGG1 upregulates VECAM (Vascular cell adhesion protein) more than LGG2 with or without cytokine stimulation. In contemplated embodiments, immune recruitment will be tested as a function of bacteria strain co-culture in embodiments of intestine on-chip. FIG. 47D.

FIG. 47A-D shows exemplary Lactobacillus upregulated Endothelial Selectin Expression. Gene Expression Measured by qPCR. Control (no bacteria); Lactobacilli Strains LGG1 vs. LGG2 with or without TNF-α/IL-1β. Log2 (fold-challenge). FIG. 47A ICAM. FIG. 47B MADCAM. FIG. 47C PECAM. FIG. 47D VECAM.

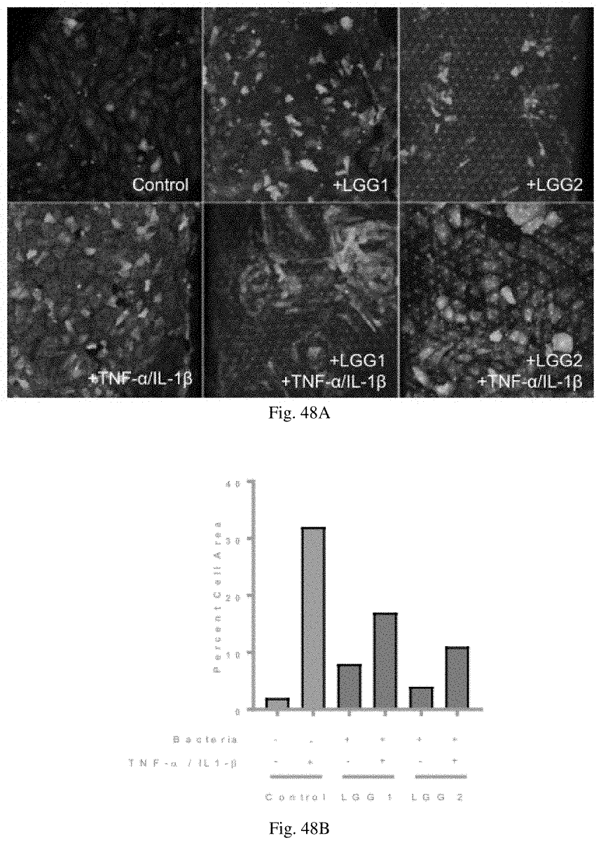

ICAM selectin expression is upregulated by Lactobacclli. Further, ICAM is increased by contact with Lactobacilli Strains LGG1 vs. LGG2 with or without TNF-α/IL-1β as shown by Immunofluorescence of Endothelial ICAM Selectin Expression. FIG. 48A. Image Quantification of ICAM Expression is shown in FIG. 48B.

FIG. 48A-B shows exemplary Lactobacillus upregulated Endothelial Selectin Expression. FIG. 48A Immunofluorescence of Endothelial ICAM Selectin Expression. FIG. 48B Image Quantification of ICAM Expression. Control (no bacteria); Percent cell area.

Cross-sectional images of the Intestine-Chip with lactobacilli strains reveal a more developed epithelial morphology.

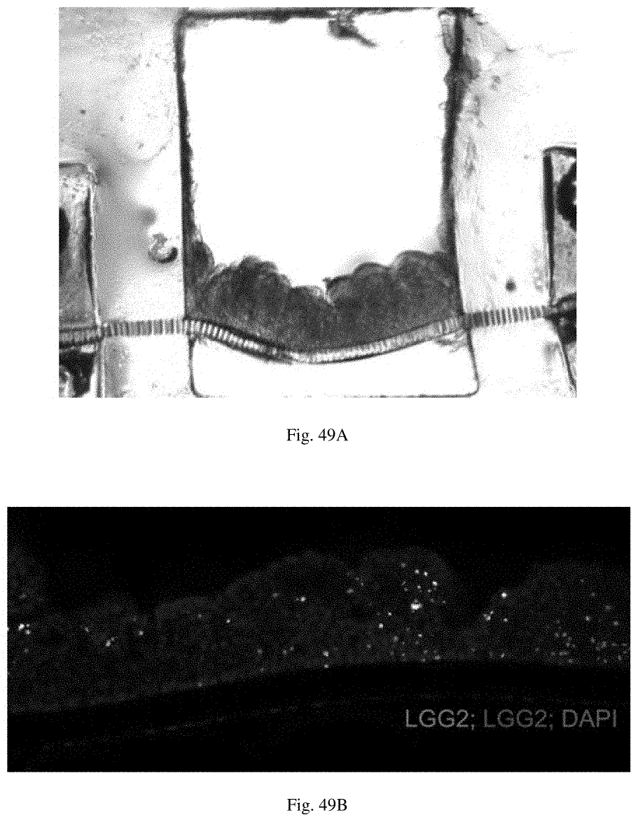

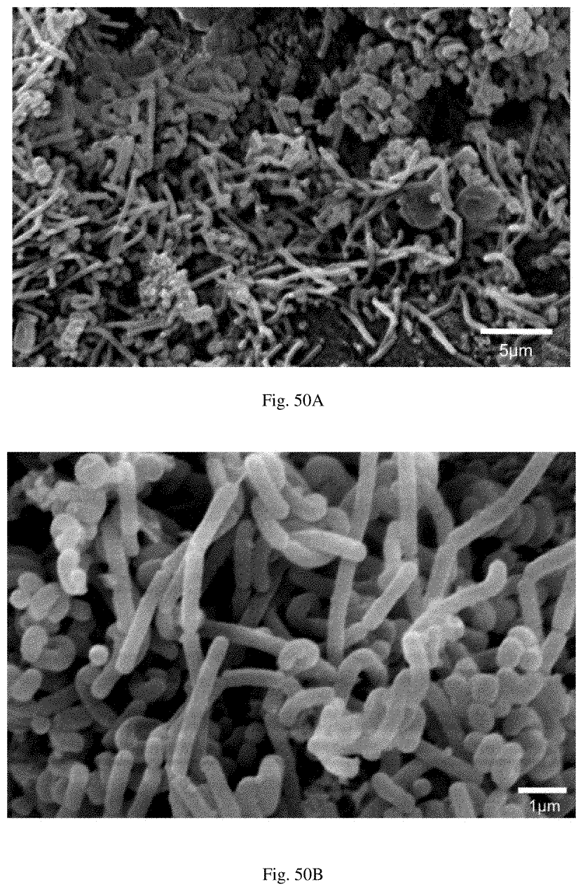

FIGS. 49A-B shows exemplary representative bright-field (FIG. 49A) and immunofluorescent images (FIG. 49B) of Intestine-Chip cross-sections. FIG. 49A bright-field micrograph Cross-Section of a +LGG1 contacted chip. FIG. 49B immunofluorescent micrograph of a +LGG2 contacted chip, green LGG2, red LGG2 and nuclei DAPI colored blue.

The probiotic function of LGG is mediated by mucosal attachment. SEM images of the Intestine-Chip show evidence of LGG attachment and biofilm formation.

FIGS. 50A-B shows exemplary Scanning Electron Micrographs of LGG on the Intestine-Chip. FIG. 50A bar=5 μm. FIG. 50B higher power image. bar=1 μm.

6. Additional LGG Data.

The presence of LGGs have a significant effect on cytokine production from the epithelium. LGG1 generally decreases the epithelial pro-inflammatory response.

FIG. 66 shows exemplary LGG1 vs. LGG2 bacterial modulation of cytokine response as a profile of cytokine production.

IL-8 had a large response in the treatment conditions tested; however, the measured values were beyond the instruments limits of detection. Thus, further experiments will use diluted samples for IL-8 measurements.

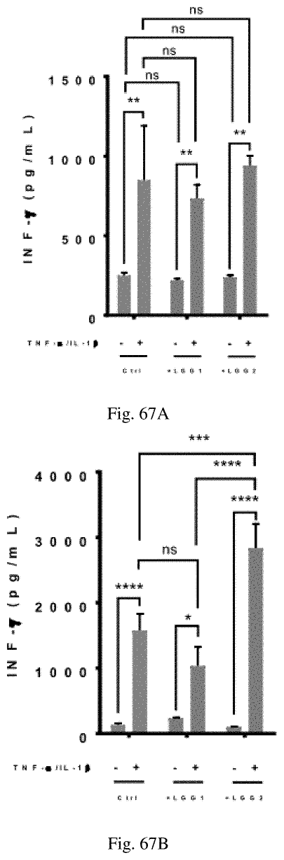

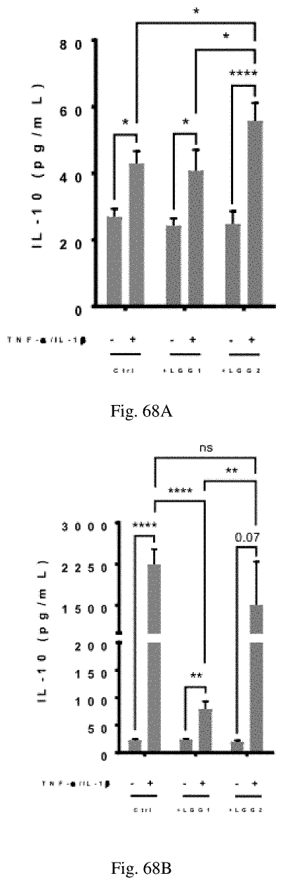

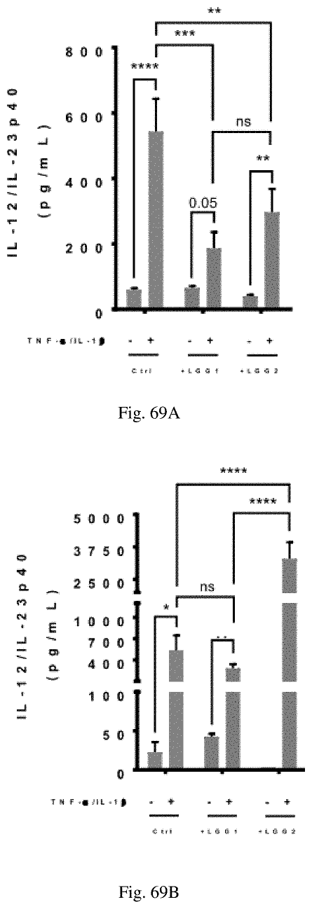

FIGS. 67A-B shows exemplary LGG1 vs. LGG2 bacterial modulation of cytokine response. Control (no bacteria); LGG1 vs. LGG2; with or without TNF-α/IL-1β. FIG. 67A IL-8 cytokine production at 4 hrs post-treatment. FIG. 67B IL-8 cytokine production at 4 hrs post-treatment.

LGG2 induces increased INF-γ production after 14 hrs of TNF-α/IL-1β challenge.

FIGS. 68A-B shows exemplary LGG1 vs. LGG2 bacterial modulation of cytokine responses between controls (no bacteria); LGG1 vs. LGG2; with or without TNF-α/IL-1β. FIG. 68A INF-γ (IFN-gamma) cytokine production at 4 hrs post-treatment. FIG. 68B INF-γ cytokine production at 14 hrs post-treatment.

After 14 hrs of TNF-α/IL-1β challenge, IL-10 production is lower in the presence of LGG1 and higher with LGG2.

FIGS. 69A-B shows exemplary Bacterial Modulation of Cytokine Response. Control (no bacteria); LGG1 vs. LGG2; with or without TNF-α/IL-1β. FIG. 69A IL-10 cytokine production at 4 hrs post-treatment. FIG. 69B IL-10 cytokine production at 14 hrs post-treatment.

LGG2 stimulates increased IL-12/IL-23p40 production after 14 hrs of TNF-α/IL-1β challenge.

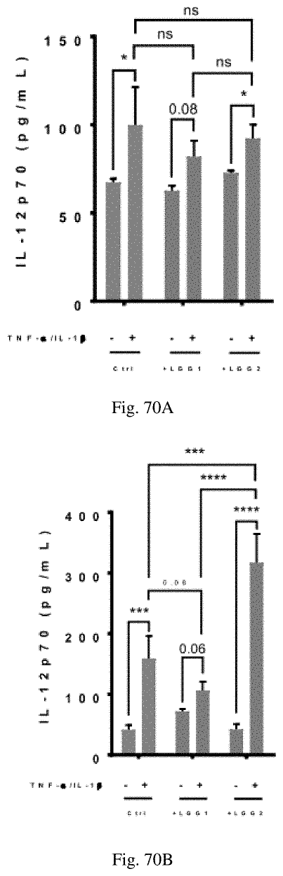

FIGS. 70A-B shows exemplary LGG1 vs. LGG2 bacterial modulation of cytokine responses between controls (no bacteria); LGG1 vs. LGG2; with or without TNF-α/IL-1β. FIG. 70A IL-12/IL-23p40 cytokine production at 4 hrs

LGG2 stimulates increased IL-12p70 production after 14 hrs of TNF-α/IL-1β challenge.

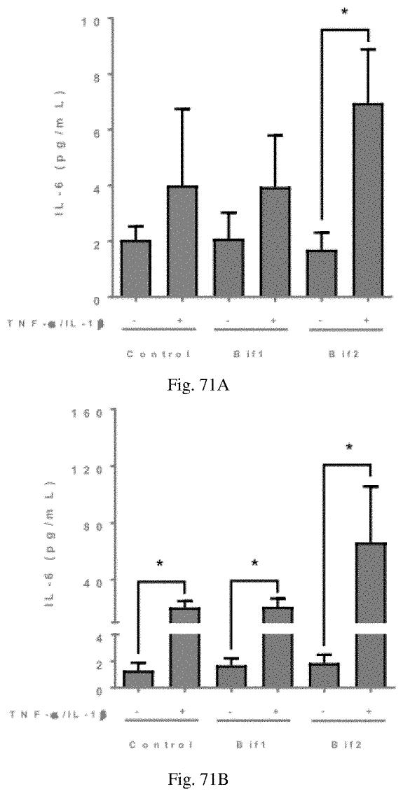

FIGS. 71A-B shows exemplary LGG1 vs. LGG2 bacterial modulation of cytokine responses between controls (no bacteria); (no bacteria); LGG1 vs. LGG2; with or without TNF-α/IL-1β. FIG. 71A IL-12p70 cytokine production at 4 hrs post-treatment. FIG. 71B IL-12p70 cytokine production at 14 hrs post-treatment.

7. Bifidobacteria (Bif) Bacteria Strains Comparative Results Between Two Strains.

Bifidobacterium spp. (including but not limited to several strains of Bif) (formally regarded as a type of lactobacillus now designated as a different genius). Features include but are not limited to: Extensive published human clinical data for reference; thrives in an anaerobic microenvironment; has a unique carbohydrate metabolism; and produces fermentation product(s) linked to probiotic functions. However, it was surprisingly found that this organism would also grow in an aerobic microenvironment on one embodiment of a microfluidic intestine on-chip.

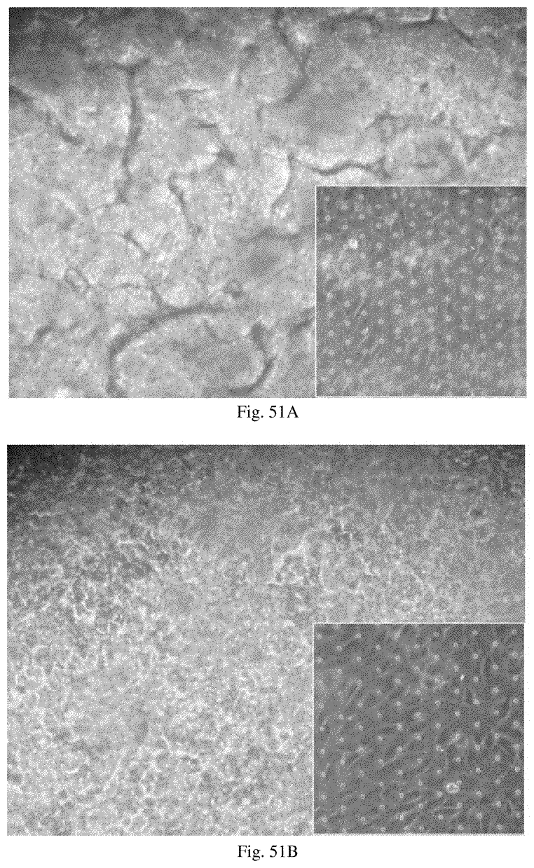

FIGS. 51A-B shows exemplary Epithelial Morphology in representative bright-field images of Intestine On-Chip. FIG. 51A Control Chip (no bacteria added), Day 8. FIG. 51B Chip treated with +Bif1, Day 8.

In the presence of Bifidobacteria (Bif1), the epithelium in one embodiment of an Intestine On-Chip maintained a monolayer-type architecture instead of forming typical villa. These methods included a 5% cysteine solution, which may have damaged the epithelium.

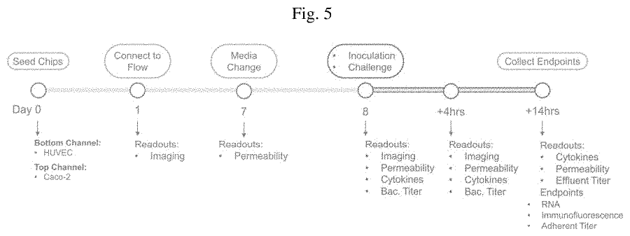

Therefore, a slightly different experimental plan, i.e. timeline, was designed for use without a cysteine solution. In one embodiment of an intestine on-chip, on Day 0, the bottom channel was seeded with HUVECs, while the top channel was seeded with Caco-2 cells. See additional timeline details in FIG. 5.

FIG. 5 shows an exemplary contemplated experimental Plan and Timeline. Day 0: Seed Chips. Bottom Channel: HUVEC. Top Channel: Caco-2. Day 1: Connect to flow. Readouts: Imaging; etc. Day 3: Inoculation and Readouts: Imaging; Permeability; Bacterium Titer; etc. Day 8: Inflammatory Challenge. Readouts: Imaging; Permeability; Cytokines; Bacterium Titer; etc. Day 8 +4 hrs: Readouts: Imaging; Permeability; Cytokines; Bacterium Titer; etc. Day 8 +14 hrs: Readouts: Permeability; Cytokines; Effluent Titer; etc. Day 8 +14 hrs: Collect Endpoints: RNA; Immunofluorescence; Adherent Titer; etc.

TABLE 2
Exemplary Contemplated Testing Conditions. No cysteine reducing
agent was used in these embodiments of an Intestine On-Chip.

| Strain | Treatment | |
|---|---|---|
| | 1. | Control |
| | 2. | +TNFα/IL-1β (30 ng/mL) |
| +Bif1 | 1. | Control |
| | 2. | +TNFα/IL-1β (3(30 ng/mL) |
| +Bif2 | 1. | Control |
| | 2. | +TNFα/IL-1β (30 ng/mL) |

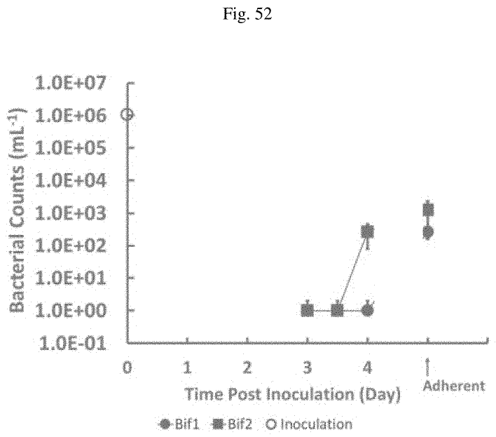

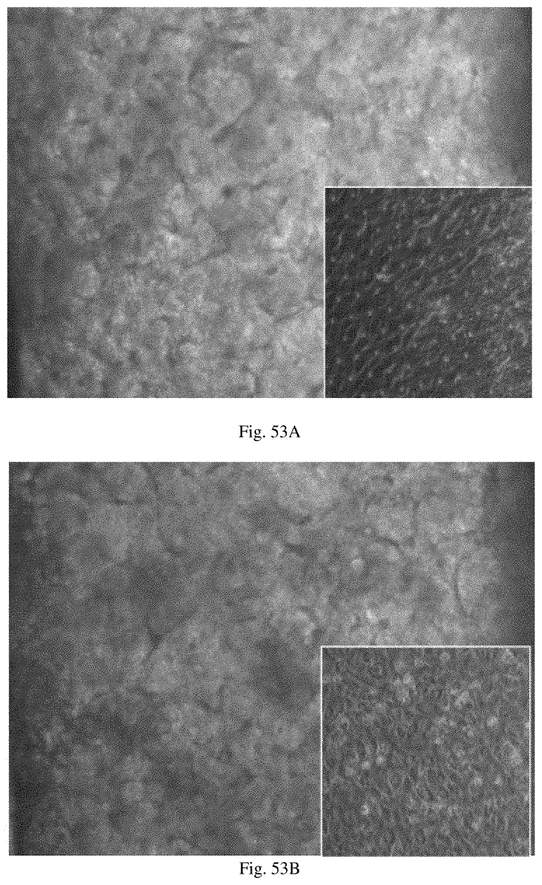

FIG. 52 shows an exemplary aerotolerant Bifidobacteria Stably Colonize the Intestine-Chip. Bacterial Titration. Viable, aerotolerant bifidobacterial strains colonized the Caco-2 Intestine-Chip and could be detected in plate Titers.

8. Bacteria Strains: Comparative Results Between Two Strains.

No apparent change in epithelial tissue morphology with Bif1 strain.

FIGS. 53A-B shows exemplary Bifidobacterial modulation of Epithelial Morphology. Representative brightfield images of Intestine-Chip, untreated. FIG. 53A Control Chip (no bacteria added), Day 8. FIG. 53B Chip treated with +Bif1, Day 8.

No apparent change in epithelial tissue morphology upon Bif1 addition in the context of inflammation.

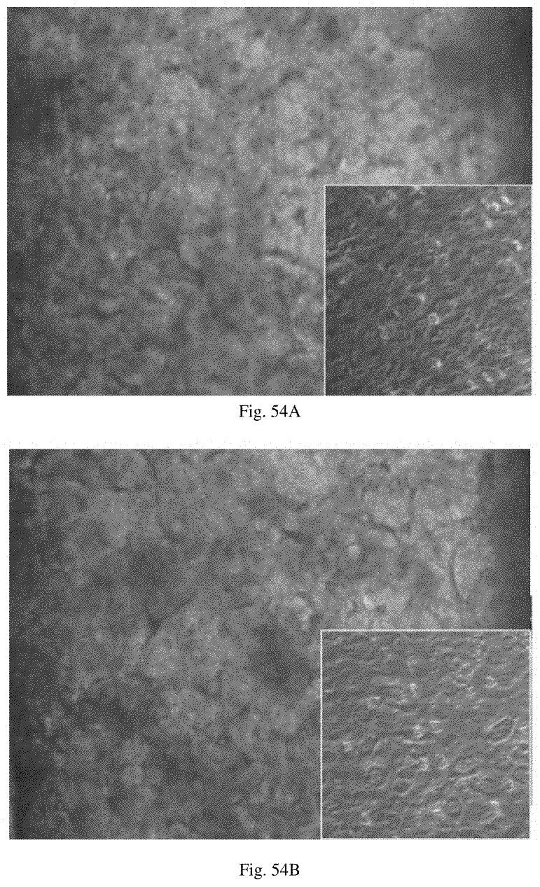

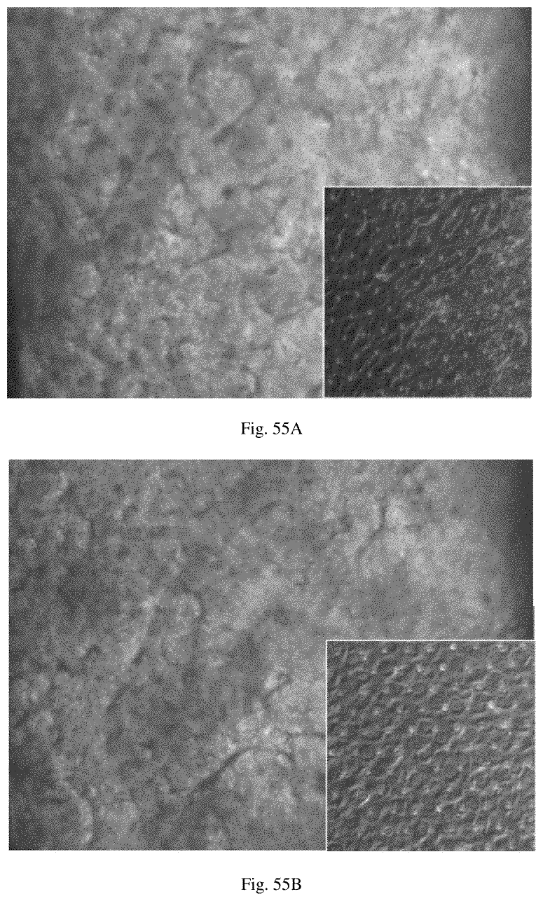

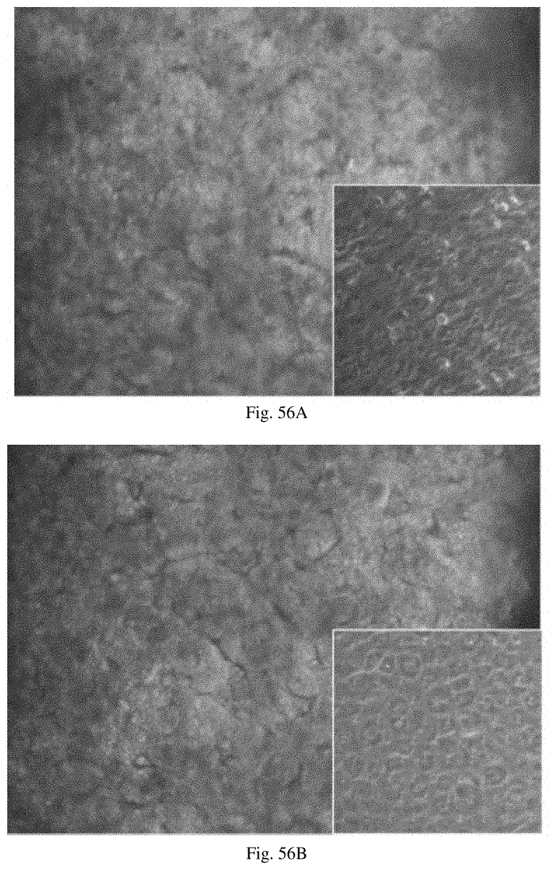

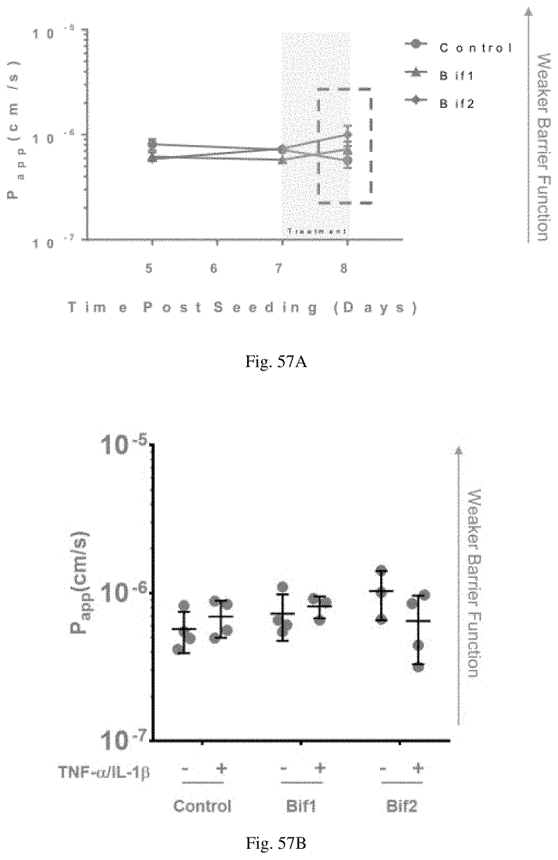

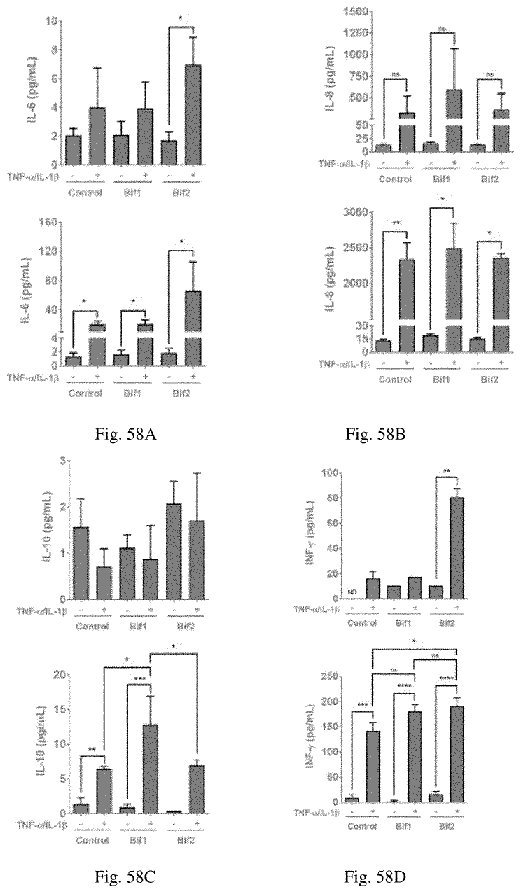

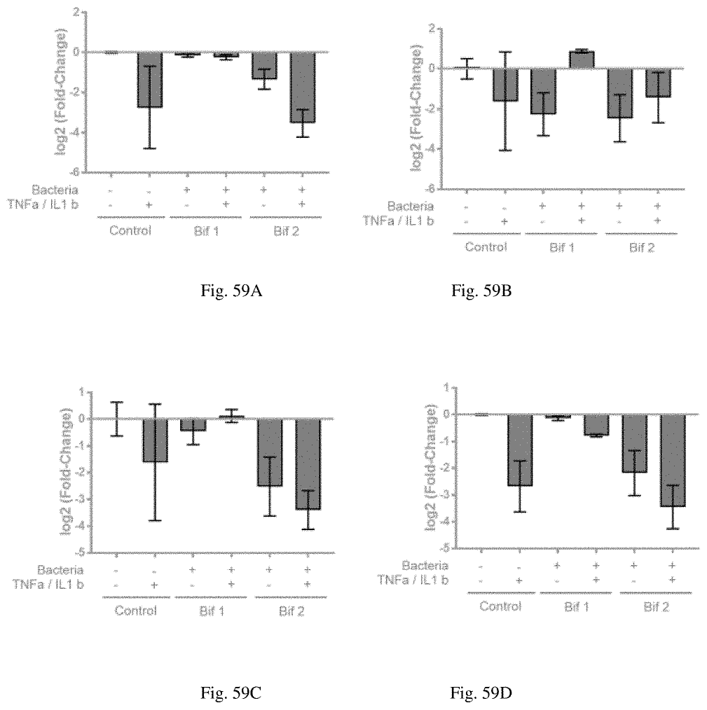

FIGS. 54A-B shows exemplary Bifidobacterial modulation of Epithelial Morphology. Representative brightfield images of Intestine-Chip, treated. FIG. 54A Control Chip Chip treated with +TNF-α/IL-1β (no bacteria added), Day 8. FIG. 54B Chip treated with +Bif1 +TNF-α/IL-1β, Day 8.

No apparent change in epithelial tissue morphology with Bif2 strain.

FIGS. 55A-B shows exemplary Bifidobacterial modulation of Epithelial Morphology. Representative brightfield images of Intestine-Chip, untreated. FIG. 55A Control Chip treated with +TNF-α/IL-1β (no bacteria added), Day 8. FIG. 55B Chip treated with +Bif2+TNF-α/IL-1β, Day 8.

No apparent change in epithelial tissue morphology upon Bif2 addition in the context of inflammation.

FIGS. 56A-B shows exemplary Bifidobacterial modulation of Epithelial Morphology. Representative brightfield images of Intestine-Chip, treated. FIG. 56A Control Chip treated with +TNF-α/IL-1β (no bacteria added), Day 8. FIG. 56B Chip treated with +Bif2+TNF-α/IL-1β, Day 8.

9. Bifidobacteria Barrier Function.

Co-inoculation of Bifidobacteria strains does not disrupt epithelial barrier function. Higher concentrations and longer exposures to TNF-α/IL-1b are contemplated to disrupt barrier function.

FIGS. 57A-B shows exemplary Bifidobacterial modulation of Epithelial Barrier Function that does not appear to significantly change by day 8-post infection. FIG. 57A Apparent Permeability over time between control (no bacteria), Bif1 and Bif2. FIG. 57B Comparative apparent Permeability on Day 8 (14 hours post treatment).

10. Bifidobacteria Secreted Cytokines.

Co-culture with Bifido strains does not significantly affect baseline inflammatory state. However, the pro-inflammatory response is increased in the presence of Bif2.

FIGS. 58A-D shows exemplary secreted cytokines. Bifidobacterial modulation of epithelial cytokine responses. FIG. 58A Secreted IL-6 (pg/ml) post-treatment 4 hr (top graph) post-treatment 14 hr (bottom graph). FIG. 58B Secreted IL-8 (pg/ml) post-treatment 4 hr (top graph) post-treatment 14 hr (bottom graph). FIG. 58C Secreted IL-10 (pg/ml) post-treatment 4 hr (top graph) post-treatment 14 hr (bottom graph). FIG. 58D Secreted IFN-gamma (pg/ml) post-treatment 4 hr (top graph) post-treatment 14 hr (bottom graph).

Bifidobacteria species affect expression of critical epithelial tight junction proteins.

FIGS. 59A-D shows exemplary Bifidobacterial modulation of Epithelial Tight Junctions. Bif1 vs. Bif2 in relation to control (no bacteria); with and without TNF-α/IL-10. Gene Expression Measured by qPCR. FIG. 59A shows exemplary expression of Claudin 1. FIG. 59B shows exemplary expression of Claudin 2. FIG. 59C shows exemplary expression of Claudin 3. FIG. 59D shows exemplary expression of Occludin.

11. Bifidobacteria Immunomodulation.

Bifidobacteria induces changes in epithelial signaling to the innate and adaptive immune systems as well as secretion of factors that regulate bacterial proliferation in the lumen.

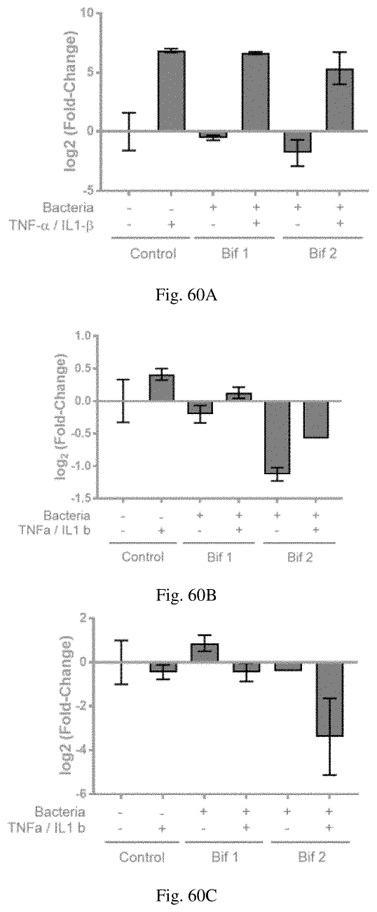

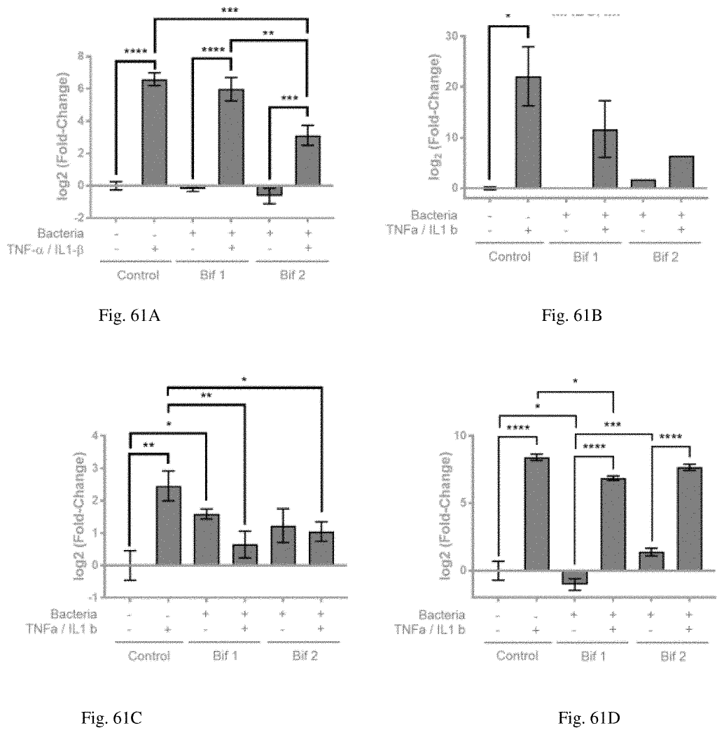

FIGS. 60A-C shows an exemplary Bifidobacterial modulation of epithelial inflammatory processes. Bif1 vs. Bif2 in relation to control (no bacteria); with and without TNF-α/IL-1β. Gene Expression Measured by qPCR. FIG. 60A shows exemplary expression of IL-8. FIG. 60B shows exemplary expression of S100A9. FIG. 60C shows exemplary expression of epithelial-cell—derived cytokine thymic stromal lymphopoietin (TSLP).

12. Bifidobacteria Selectin Expression.

Bifidobacteria are able to communicate and influence selectin expression via the host epithelium. Expect significant changes in immune recruitment.

FIGS. 61A-D shows an exemplary Bifidobactericil modulation of endothelial selectin expression. Bif1 vs. Bif2 in relation to control (no bacteria); with and without TNF-α/IL-1β. Gene Expression Measured by qPCR. FIG. 61A shows exemplary expression of ICAM. FIG. 61B shows exemplary expression of MADCAM. FIG. 28C shows exemplary expression of PCEAM. FIG. 61D shows exemplary expression of VECAM.

ICAM selectin expression is downregulated by Bifidobacteria.

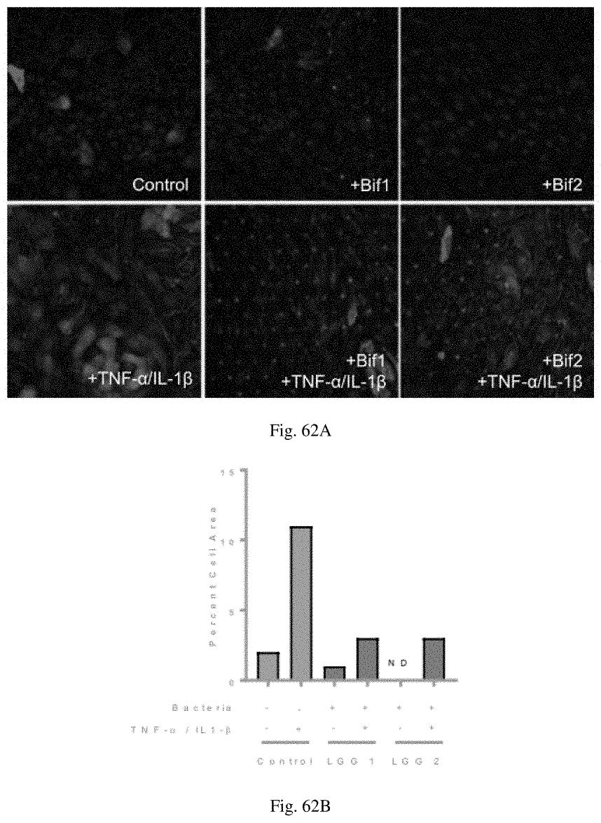

FIGS. 62A-B shows exemplary immunofluescent immunostained micrograghs and charts demonstrating that Bifidobacteria downregulated endothelial selectin expression. FIG. 62A Immunofluorescence of ICAM Selectin Expression. Control (no bacteria); +Bif1 +Bif2; with or without TNF-α/IL-1β. FIG. 62B Quantification of ICAM Expression; Percent cell area. Control (no bacteria); +Bif1 Bif2 with or without TNF-α/IL-1β.

Proliferation rates of Bif 1 vs. Bif2 strains were significantly different. Bif 1 is less aerotolerant and may require the presence of a reducing agent. Bif2 glycerol stock may contain multiple clones. However, no viable Bifido colonies were recovered from the Intestine-Chip in the absence of reducing agent. Contemplated post-chip sample handling and longer agar incubation times contributed to the absence of viable bacteria. Future experiments are contemplated to test the epithelium at lower cysteine concentrations. Including 0.5% as used in media cultures.

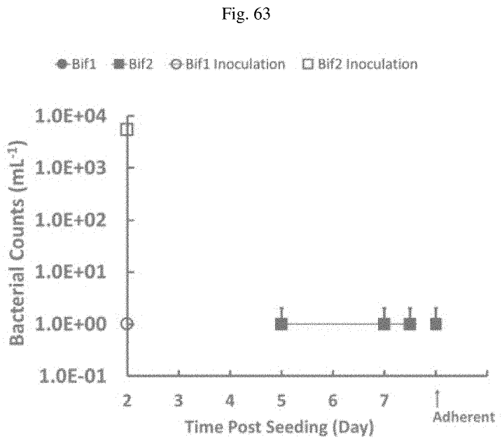

FIG. 63 shows exemplary Bacterial Titration over time collected from one embodiment of a microfluidic intestine on-chip. Viable Bifidobacteria were not Recovered from one embodiment of an Intestine On-Chip.

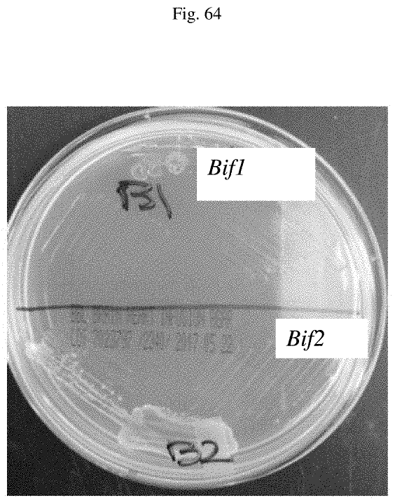

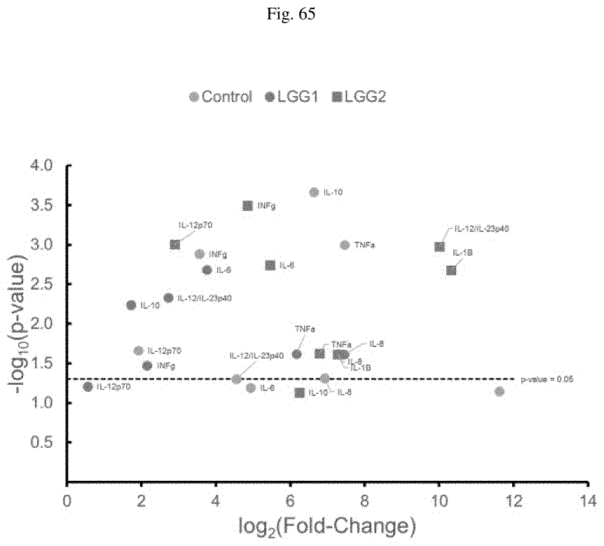

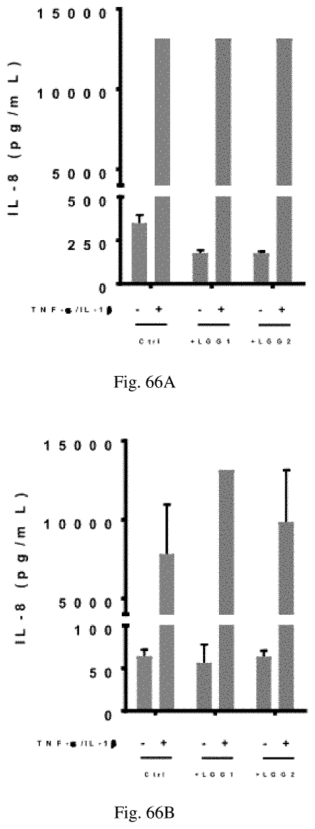

FIG. 64 shows exemplary Bifido Strain Colony Streaks on Brain Heart Infusion Agar. Bif1 Strain above black line, Bif1 strain below black line. Bif1 Strain Requires Reducing Environment.

TABLE 3
Optical Density of Overnight Cultures in MRS Media.

| | | |
|---|---|---|
| 0.05% Cysteine | Anaerobic | OD600 |

| (W/V) | Incubation | Bif1 | Bif2 |
|---|---|---|---|
| − | − | 0.0 | 0.0 |
| + | − | 1.5 | 2.0 |
| − | + | 0.0 | 2.1 |
| + | + | 2.0 | 2.5 |

13. Additional Bifidobacteria Data.

FIG. 71A-B shows an exemplary Bifidobacterial modulation of cytokine responses between controls (no bacteria); Bif1 vs. Bif2; with or without TNF-α/IL-1β. FIG. 71A IL-6 cytokine production at 4 hrs post-treatment. FIG. 71B IL-6 cytokine production at 14 hrs post-treatment.

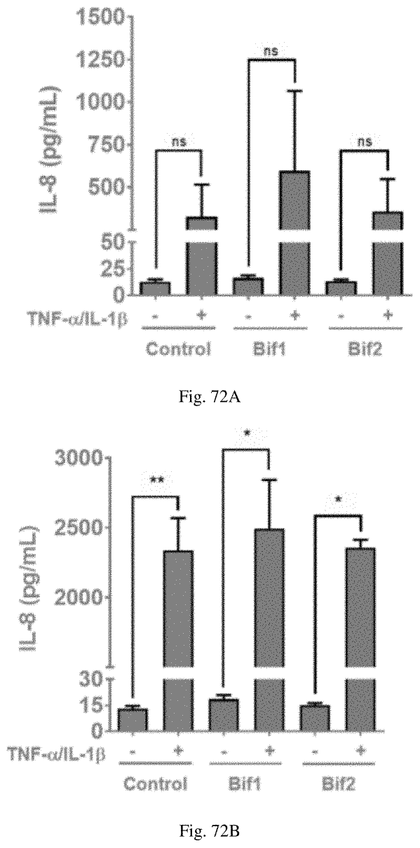

FIGS. 72A-B shows an exemplary Bifidobacterial modulation of cytokine responses between controls (no bacteria); Bif1 vs. Bif2; with or without TNF-α/IL-1β. FIG. 72A IL-8 cytokine production at 4 hrs post-treatment. FIG. 72B IL-8 cytokine production at 14 hrs post-treatment.

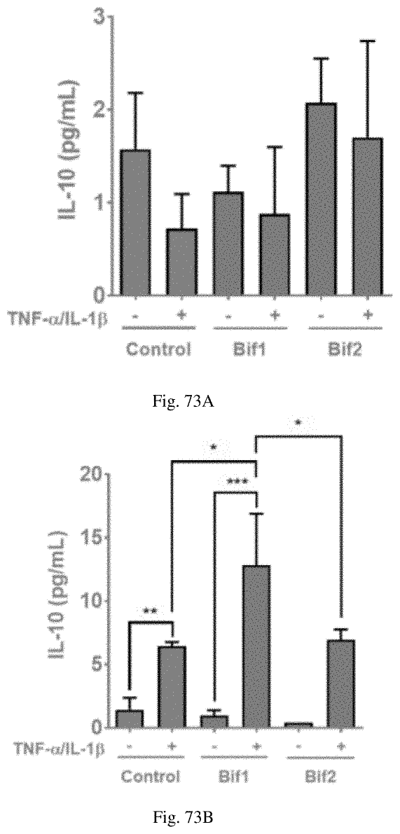

FIGS. 73A-B shows an exemplary Bifidobacterial modulation of cytokine responses between controls (no bacteria); Bif1 vs. Bif2; with or without TNF-α/IL-1β. FIG. 73A IL-10 cytokine production at 4 hrs post-treatment. FIG. 73B IL-10 cytokine production at 14 hrs post-treatment.

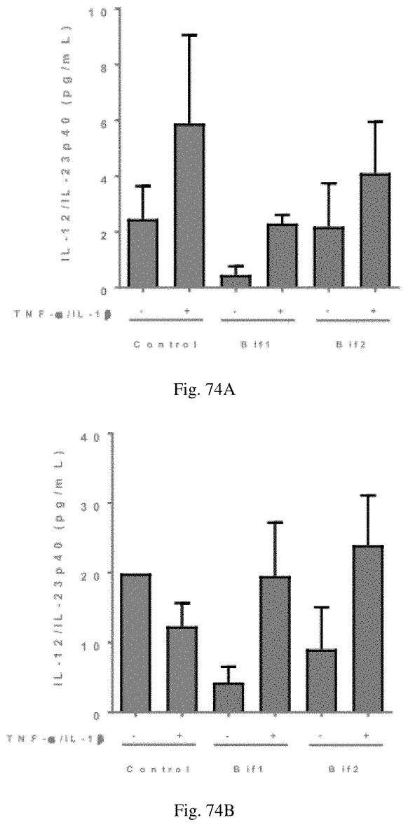

FIGS. 74A-B shows an exemplary Bifidobacterial modulation of cytokine responses between controls (no bacteria); Bif1 vs. Bif2; with or without TNF-α/IL-1β. FIG. 41A IL-12/IL-23p40 cytokine production at 4 hrs post-treatment. FIG. 41B IL-12/IL-23p40 cytokine production at 14 hrs post-treatment.

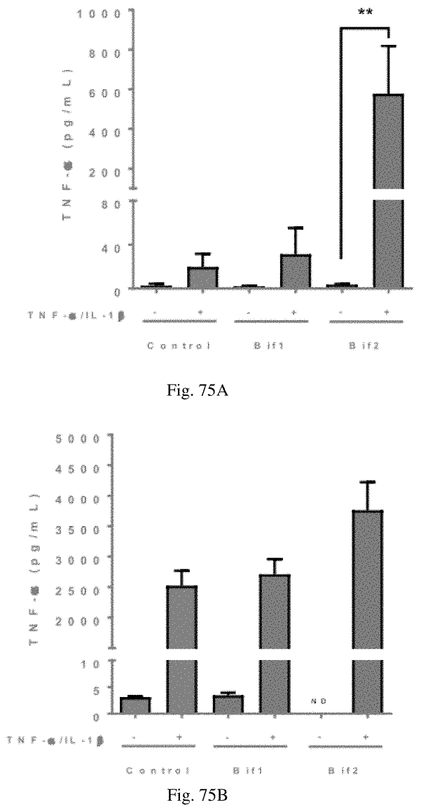

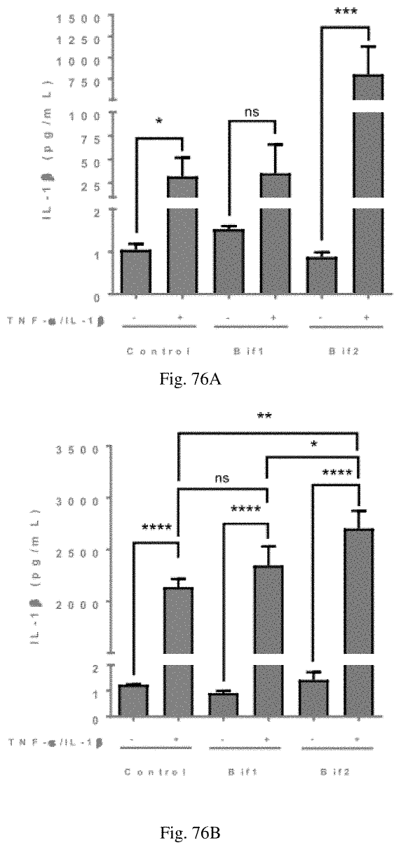

FIGS. 75A-B shows an exemplary Bifidobacterial modulation of cytokine responses between controls (no bacteria); Bif1 vs. Bif2; with or without TNF-α/IL-1β. FIG. 75A TNF-α cytokine production at 4 hrs post-treatment. FIG. 75B TNF-α cytokine production at 14 hrs post-treatment.

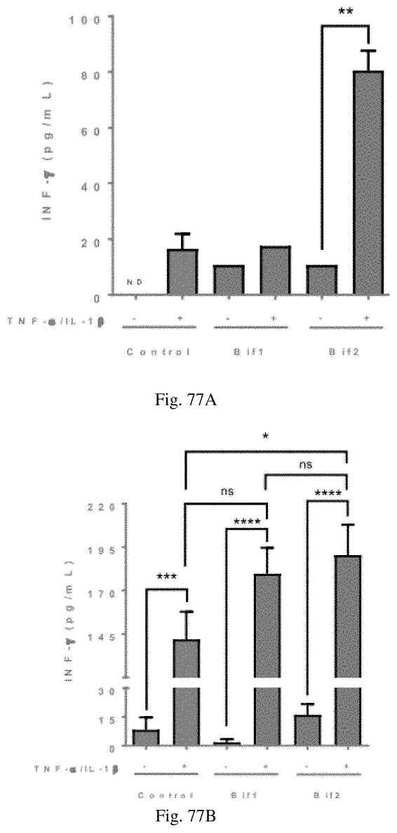

FIGS. 76A-B shows an exemplary Bifidobacterial modulation of cytokine responses between controls (no bacteria); Bif1 vs. Bif2; with or without TNF-α/IL-1β. FIG. 76A IL-1β cytokine production at 4 hrs post-treatment. FIG. 76B IL-1β cytokine production at 14 hrs post-treatment.

FIGS. 77A-B shows an exemplary Bifidobacterial modulation of cytokine responses between controls (no bacteria); Bif1 vs. Bif2; with or without TNF-α/IL-1β. FIG. 77A IFN-gamma cytokine production at 4 hrs post-treatment. FIG. 77B IFN-gamma cytokine production at 14 hrs post-treatment.

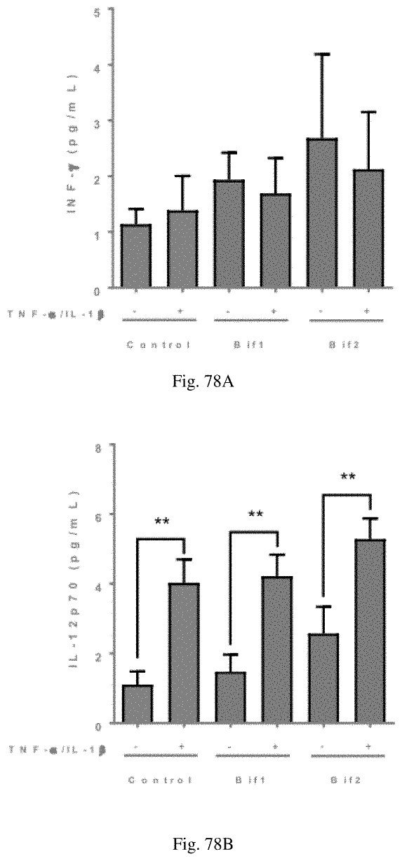

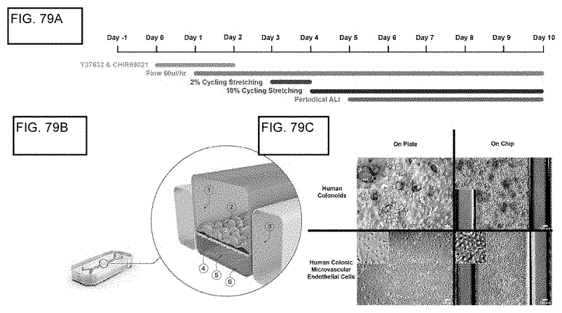

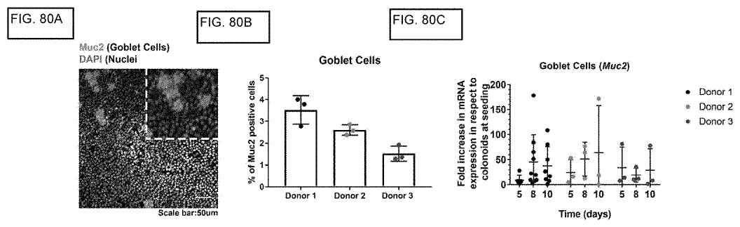

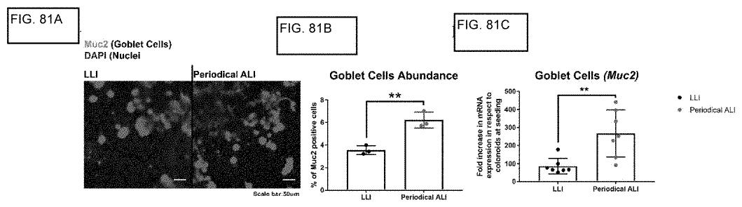

FIGS. 78A-B shows an exemplary Bifidobacterial modulation of cytokine responses between controls (no bacteria); Bif1 vs. Bif2; with or without TNF-α/IL-1β. FIG. 78A IFN-gamma cytokine production at 4 hrs post-treatment. FIG. 78B IL-12p70 cytokine production at 14 hrs post-treatment.

14. Summary and Conclusions.

An exemplary screen of 4 different bacterial species on the Intestine-Chip demonstrated the presence of commensals had a significant impact on the production of inflammatory cytokines contemplated to modulate the mucosal immune response.

Contemplated challenges to overcome include but are not limited to the discovery that a 5% cysteine concentration affected infection of the Intestine On-Chip epithelium. While Bifidobacteria modulated the epithelial response without cysteine; no viable bacterial colonies were recovered. Permeability was not perturbed with the TNF-α/IL-1β challenge amounts used herein. Thus, an increased cytokine dose and duration of treatment is contemplated for use with modeling infection of Intestine On-Chip epithelium.

C. Bacteria On Other Cell Types.

In fact, we showed that it is possible to infect the Airway Chip with bacterial pathogens for several days. Exemplary readouts after infection include but are not limited to: real time imaging; mucociliary activity assays; immunofluorescence analysis of cell markers; cytokine analysis from effluent collections; RNA analysis of airway cells; microbial counts in effluent; microbial counts in cell samples; microbial counts of stained (direct dyes or antibodies conjugated to dyes) under real time imaging; immunofluorescenc etc.

For one example, pneumococcus is a common cause of bacterial pneumonia, an illness that can be particularly serious in a person with asthma. Thus, in one embodiment, respiratory cells with a disease phenotype (e.g. asthma, COPD, CF) may be exposed “on-chip” to pneumococcus bacteria.

In another example, Group A streptococcus bacteria associated with strep throat infections may be used in place of or in combination with a respiratory virus in an Airway chip. Thus, in one embodiment, respiratory cells with a disease phenotype (e.g. asthma, COPD, CF) may be exposed “on-chip” to Group A streptococcus bacteria. In yet another example, a Mycobacterium tuberculosis (TB) bacteria may be used in an Airway chip. Thus, in one embodiment, respiratory cells with a disease phenotype (e.g. asthma, COPD, CF) may be exposed “on-chip” to Mycobacterium tuberculosis (TB) bacteria.

Moraxella spp.

Respiratory pathogens involved in exacerbation, i.e. a severe phenotype, include Moraxella spp. Thus, in another example, Moraxella spp. such as Moraxella catharallis may infect a microfluidic Airway Chip, e.g. a microfluidic asthma Airway Chip; a microfluidic CF Airway Chip; a microfluidic COPD Airway Chip, etc., inducing a severe phenotype on-chip. Thus, in one embodiment, respiratory cells with a disease phenotype (e.g. asthma, COPD, CF) may be exposed “on-chip” to Moraxella spp. bacteria. Isolates of Moraxella catharallis (MC) tested included a MC ATCC strain compared to a MC clinical isolate strain. In some embodiments, these bacteria are found inside (intracellular) the epithelial cells following infection of a microfluidic Airway Chip.

Therefore, Moraxella catharallis bacteria are found inside the epithelial cells following infection of a microfluidic Airway Chip. An exemplary confocal image of the infected Airway chip shows intracellular staining (green). For comparison, transwells infected with 10⁶ CFU per transwell, at a MOI of 10, have no observable stained bacteria.

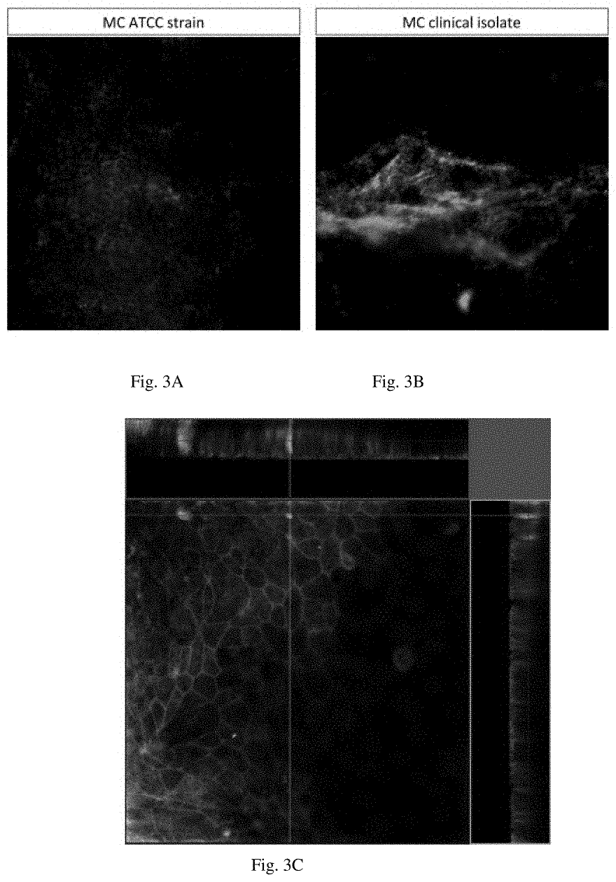

FIGS. 3A-C shows exemplary micrographs of a bacteria strain, Moraxella catharalis (MC), that acts as an exacerbator and can induce biofilm formation on top of epithelial cells after infecting one embodiment of a microfluidic Airway On-Chip. 10⁶ CFU per chip. We showed that it is possible to infect the Airway Chip with bacterial pathogens for several days.

FIG. 3A MC ATCC strain.

FIG. 3B MC clinical isolate strain. MOI 10 24 hours post-infection (hpi).

FIG. 3C shows a confocal immunostained fluorescent micrograph including Z-stacks across the top and along the right hand side of the image, pink epithelial junction marker, green bacteria and blue DAPI stained nuclei. Respiratory pathogens involved in exacerbation include Moraxella catharallis. The upper (or right side on the side bar) part of the Z-stacks represent apical regions then down through the cells to the basil regions at the bottom of bar (or left side of the side bar). These Z-stacks indicate that bacterium are intracellularly located. Therefore, Moraxella catharallis bacteria are found inside the epithelial cells following infection of a microfluidic Airway Chip. This exemplary confocal image of the infected Airway chip shows intracellular staining (green). For comparison, transwells infected with 10⁶ CFU per transwell, at a MOI of 10, have no observable stained bacteria.

Pseudomonas spp.

In another example, Pseudomonas spp. such as Pseudomonas (P.) aeruginosa (PA) may infect a microfluidic Airway Chip, e.g. a microfluidic asthma Airway Chip; a microfluidic CF Airway Chip; a microfluidic CF Airway Chip, etc., inducing a severe phenotype on-chip. Thus, in one embodiment, respiratory cells with a disease phenotype (e.g. asthma, COPD, CF) may be exposed “on-chip” to Pseudomonas spp. bacteria.

At least two strains of Pseudomonas aeruginosa are used in this study: MB5919 WT efflux competent strains; and a MB5919 derivative MB5890 multi efflux-pump defective (6-pump knock-out (KO)) mutant strain. See for details, Balibar and Grabowicz, “Mutant Alleles of lptD Increase the Permeability of Pseudomonas aeruginosa and Define Determinants of Intrinsic Resistance to Antibiotics.” Antimicrob. Agents Chemother. 60:845-854, 2016. In this 2016 publication, both strains are transfected with plasmids for disrupting LptD, a β-barrel transmembrane transport protein, the final protein involved in Lipopolysaccharides (LPS) transfer of Lipid A of LPS into the outer membrane of gram-negative bacteria. Lipopolysaccharide (LPS) refers to a main component of the outer membrane of Gram-negative bacteria, which provides a barrier for hydrophobic drugs. Both mutations lead to the deletion of a alpha-helical loop which increases extracellular access of antibiotics into the lumen of the defective β-barrel, thus increasing susceptibility of the LPS compromised bacteria to a range of antibiotics, including imipenem.

MB5919 WT strains; and a MB5890 multi efflux-pump mutant strain that is more susceptible to antibiotics than WT strains. Efflux pumps in general allow microorganisms to regulate their internal environment by removing compounds, including but not limited to: toxic substances, e.g. drugs, antimicrobial agents; metabolites; quorum sensing signal molecules; etc. Further, these efflux systems are used by the bacterium to pump solutes out of the cell. More specifically, drug efflux is one mechanism of antimicrobial resistance found in Gram-negative bacteria. Bacterial drug efflux pumps have been classified into six families by having combinations of: number of components, number of transmembrane-spanning regions, energy source used by the pump and the types of molecules that the pump exports: (1) the ATP-binding cassette (ABC) superfamily; (2) the major facilitator superfamily (MFS); (3) the multidrug and toxic compound extrusion (MATE); (4) the small multidrug resistance (SMR) family; (5) the resistance-nodulation-division (RND) superfamily; and (6) the drug metabolite transporter (DMT) superfamily.

The major clinically relevant efflux systems in P. aeruginosa belong to the RND superfamily and are typically composed of a cytoplasmic membrane pump, a periplasmic protein and an outer membrane protein channel. Multidrug resistance (MDR) pumps play a role in the antibiotic resistance of P. aeruginosa. This microorganism presents several putative MDR efflux pump-encoding genes belonging to the RND family of bacterial transporters. Among these, MexAB-OprM, MexCD-OprJ, MexEFOprN and MexXY have been the most widely studied (ref 1). MB5919 refers to P. aeruginosa PAO1. MB5890 refers to efflux-deficient P. aeruginosa PAO1 Δ(mexAB-oprM)::FRT Δ(mexCD-oprJ)::FRT Δ(mexXY)::FRT Δ(mexJKL)::FRT Δ(mexHI-opmD)::FRT Δ(opmH):: FRT mutant (ref 2). ref 1: Virulence 4:3, 223-229; Apr. 1, 2013; ref 2: Balibar and Grabowicz, “Mutant Alleles of lptD Increase the Permeability of Pseudomonas aeruginosa and Define Determinants of Intrinsic Resistance to Antibiotics.” Antimicrob. Agents Chemother. 60:845-854, 2016.

In an exemplary but nonlimiting manner, bacteria added to epithelial cells first adhere to epithelial cells (either directly or by attaching to mucin layers); and start forming microcolonies and biofilms. Some bacteria enter spaces in between cells and some bacteria enter cells to become intracellular.

In some embodiments, these bacteria are found inside (intracellularly in) the epithelial cells following infection of a microfluidic Airway Chip. In some embodiments, these bacteria form extracellular micro-colonies/aggregates following infection of a microfluidic Airway Chip.

D. Bacteria Infection.

Adhesion assay and Bacterial Counting Protocol (Method).

Exemplary on-chip infection and analysis of surface-adherent bacteria. Bacteria from log phase cultures are collected and washed with PBS. Three (3)×10⁶ CFU/chip per strain are added to airway chips at an MOI of approximately 10. Where the multiplicity of infection or MOI refers to the ratio of microbial agents (e.g. virus, bacteria) to infection targets, in this case chips containing airway cells. Infect for 1 hour (1 hpi), wash 3× in PBS to remove nonadherent bacteria. Cells are trypsinized gently so as to avoid lysing them, therefore intracellular bacteria do not contribute to the CFU counts. Cell samples are collected, e.g. washed, from chips then vortexed to disassociate cell clumps. Samples are serially diluted for CFU quantification. At least N=3 per treatment.

In order to quantify both extracellular and intracellular bacteria concurrently from a single chip, after infection unattached cells in the inoculum is removed, wash 3× with PBS and lyse cells (but not bacteria) with 1% triton. This allows counting surface-associated and intracellular bacteria cell numbers.

In order to quantify mainly intracellular bacteria, cells are treated with antibiotics to kill extracellular bacteria, antibiotics are removed, cells washed and lysed with 1% triton. The CFU counts obtained in this assay represent intracellular bacteria counts as extracellular bacteria are killed.

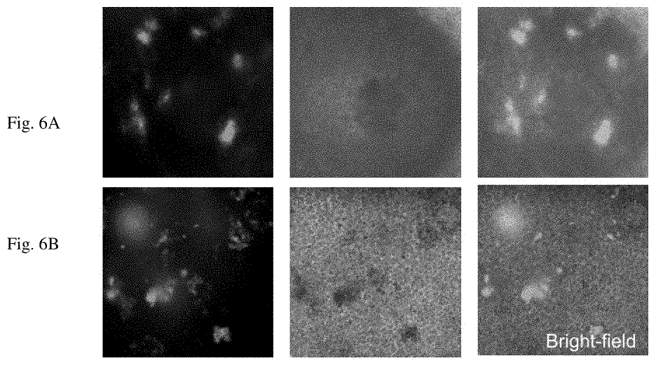

FIGS. 6A-B shows exemplary real time imaging after infection of one embodiment of an Airway Chip with bacteria P. aeruginosa infection on chip. Both pseudomonas strains, wild-type (WT) and mutant, form micro-colonies/aggregates on airway chip. Bacterial inoculum is plated and CFU are counted to ensure target MOI. Images are acquired 24 hpi. FIG. 6A PA 5919-WT. FIG. 6B PA 5890-Mutant. Left: Alexa fluor 488—anti-P. aeruginosa (Pa) antibody. Central: Bright-field. Right: Alexa fluor 488—anti-P. aeruginosa (Pa) antibody-Bright-field.

E. Exemplary On-Chip Infection Protocol.

Bacteria from log phase cultures are collected and washed with PBS.

Three (3)×10⁶ CFU/chip per strain are infected at an MOI of approximately 10. Infect for 2 h, wash 3× PBS to remove nonadherent bacteria.

Mature small airway chips at air-liquid interface (ALI) are gently washed with PBS to remove excess mucus. Cells are inoculated with 2-3×10⁶ colony forming units (CFUs) in Hank's balanced salt solution (HBSS) for 2 hours. At the end of the incubation, the inoculum is removed and the cells are washed 3× with phosphate buffer saline (PBS) to remove any nonadherent bacteria. PBS is removed and cells are incubated at ALI up to 24 hours (h) under flow in the bottom channel but not in the top channel.

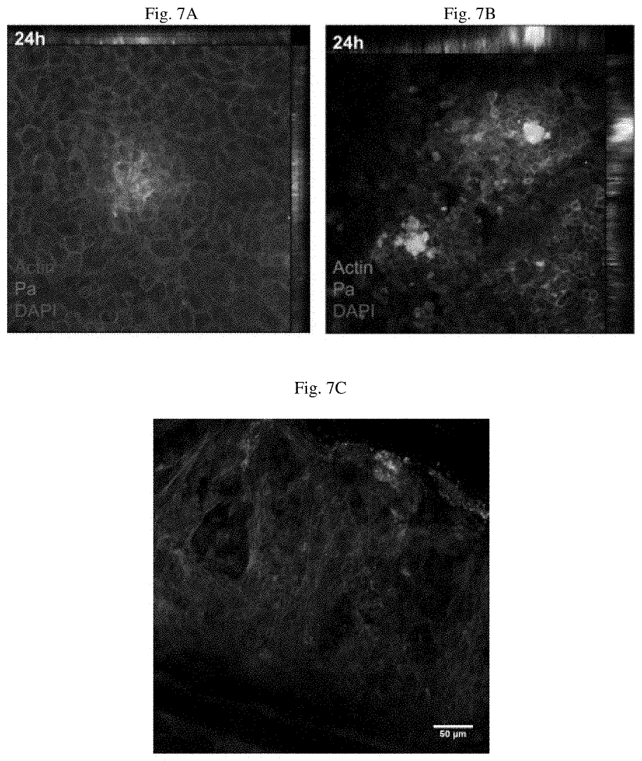

FIGS. 7A-C shows exemplary immunofluorescence, with Z-stacks or a side view, after infection of one embodiment of an Airway Chip with bacteria P. aeruginosa infection on chip. Pseudomonas establishes an intracellular niche as well as forming extracellular micro-colonies on the epithelial cell surface. Z-stacks are shown as a bar across the top (to the right of the 24 h label, and the down the right side of the micrographs. The upper (or right side on the side bar) part of the Z-stacks represent apical regions then down through the cells to the basil regions at the bottom of bar (or left side of the side bar). These Z-stacks indicate that bacterium are intracellularly located. FIG. 47A PA 5919-WT. Actin (red); Pa (green); DAPI (blue). FIG. 7B PA 5890-Mutant. Actin (pink); Pa (green); DAPI (blue). Images are acquired at 24 hpi. FIG. 7C shows a confocal immunofluorescent micrograph side view of a cell layer infected with P. aeruginosa in a microfluidic airway chip, 24 hours post infection. Actin (pink); Pa (green); DAPI (blue). Bacterial aggregates on apical surface as well as intracellular bacteria are observed.

F. Observing Changes in Mucociliary Activity, i.e. Readouts.

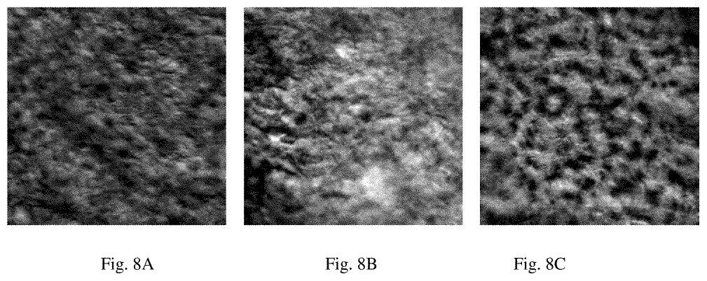

FIGS. 8A-C shows exemplary mucociliary activity photographed in bright field on one embodiment of a Pseudomonas infection on chip. Micrographs represent one image from a video of cilia beating on-chip. FIG. 8A Non-infected control microfluidic chip image representing beating cilia. FIG. 8B PA 5890-Mutant infected microfluidic chip image representing loss of beating cilia. FIG. 8C PA 5919-WT microfluidic chip image also representing a loss of beating cilia.

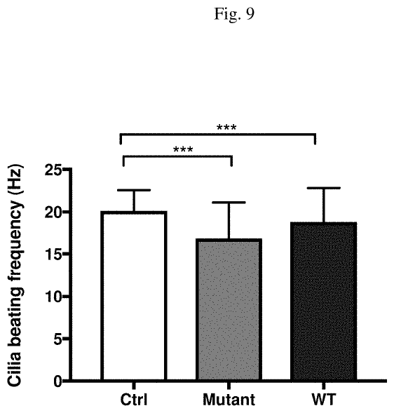

FIG. 9 shows an exemplary comparison of cilia beating frequency (CBF) between Pseudomonas strains in one embodiment of a Pseudomonas infection on chip. Images from a video of epidermal cells' cilia beating on-chip are quantitatively evaluated showing that both wild type and mutant strains has altered cilia beating frequency compared to controls without added bacteria.

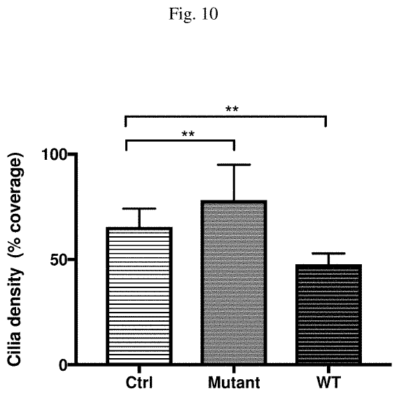

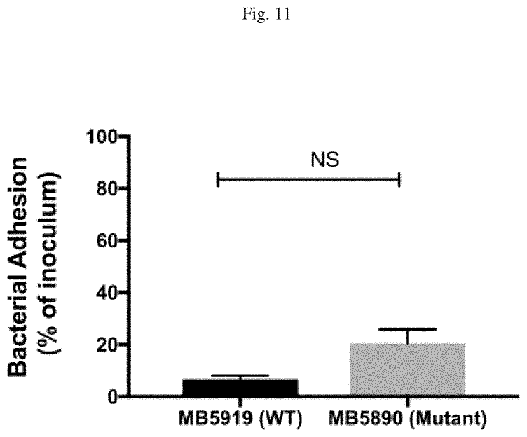

FIG. 10 an exemplary comparison of cellular cilia coverage after infection with Pseudomonas strains in one embodiment of a Pseudomonas infection on chip. Mutant (increases) and WT (decreases) show significant differences in density compared to controls.

FIG. 11 shows an exemplary Bacterial adherence on chip in one embodiment of a microfluidic airway epithelia. P. aeruginosa WT (MB5980) and mutant (MB5919) strains adhere to airway epithelium at similar rates. Unpaired t-tests p=0.0641. N=3.

G. Treatment of Microfluidic Chip with an Anti-microbial Compound.

After an exemplary on-chip infection protocol, including removing nonadherent bacteria, a test compound is immediately added to the top (apical) and bottom (basal) channels then cells are cultured under flow using media containing the anti-microbial compound, for 24 hours. Exemplary test compound amounts are at least 0.1 μg/ml, up to 50 μg/ml, up to 100 μg/ml, up to 500 μg/ml. In one embodiment, an exemplary test compound is a drug Imipenem.

More specifically, in one embodiment of an Airway on-chip, duplicate chips are infected with PA 5919 WT or PA 5890 Mutant as described herein. Then, Imipenem is added to apical and basolateral fluids (cell media) for treatment with Imipenem at 50, 100 and 500 μg/ml for 24 hours with at least one chip without Imipenem. At 24 h post treatment, wash cells 3× to remove the test drug, i.e. antibiotic Imipenem. Cells lysed with 1% Triton for 10 minutes on chip, then the lyste is collected from the chip's channels. Lysates are serially diluted for CFU quantification. N=2

Assay readouts: Viability of extracellular bacteria (apical sampling). Viability of intracellular bacteria (cell lysis and sampling). Two-way ANOVA with Dunnett's post-test **<0.05, **<0.001 (compared to untreated).

Imipenem treatment has significant bactericidal effect on MB5890 (mutant) strain and MB5919 (WT) growth. Imipenem kills most of the extracellular bacteria (this is determined by plating out apical supernatants), however does not rule out that there are still surface-associated extracellular bacteria on the epithelium. There is reduction in total bacterial counts (intra- and/or extracellular bacteria) but at this stage we cannot determine if bacteria remaining post Imipenem treatment are both intra and extracellular. However, the results clearly show P. aeruginosa killing post antibiotic treatment as indicated in CFU counts and that the bacteria can persist in small airway cells over time.

Airway cells are impermeable to Imipenem at lower concentrations. P. aeruginosa can persist in small airway cells over time when treated with 50 μg/ml imipenem.

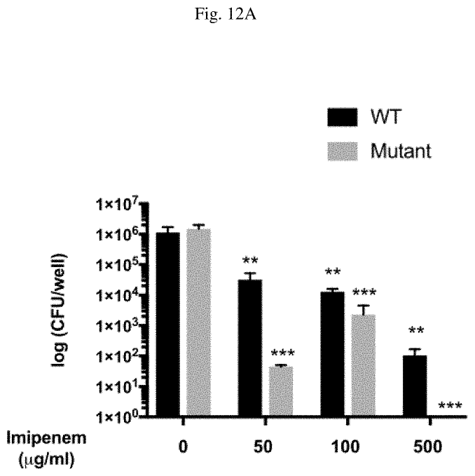

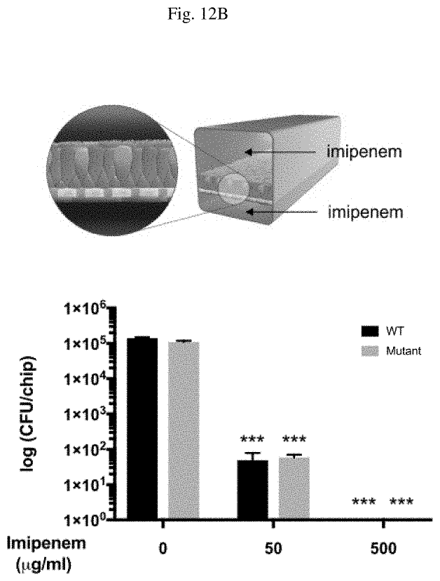

FIGS. 12A-B shows an exemplary Imipenem (Merck compound) effects on P. aeruginosa infection. FIG. 52A shows exemplary Imipenem (Merck compound) effects on P. aeruginosa infection in a Transwell culture. FIG. 12B Imipenem treatment reduces total bacterial counts via bacterial killing in one embodiment of a P. aeruginosa infection on chip. Two-way ANOVA with Dunnett's post-test **<0.05, **<0.001 (compared to untreated).

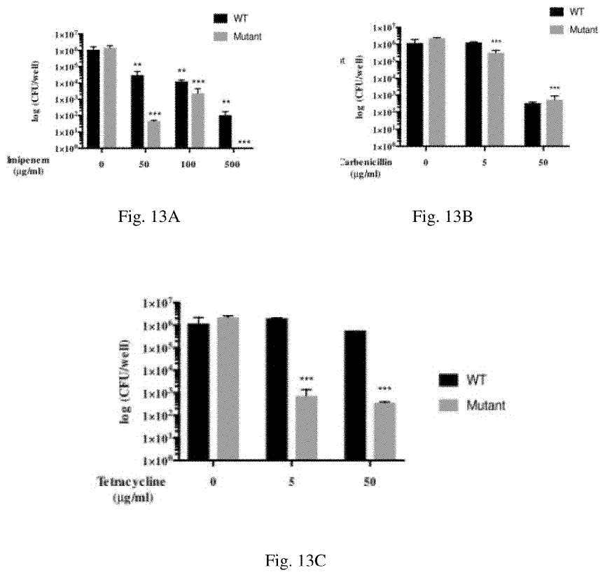

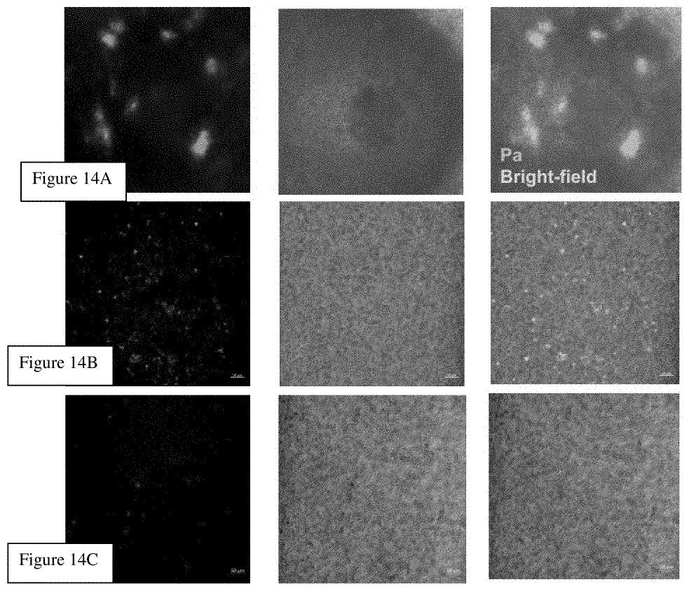

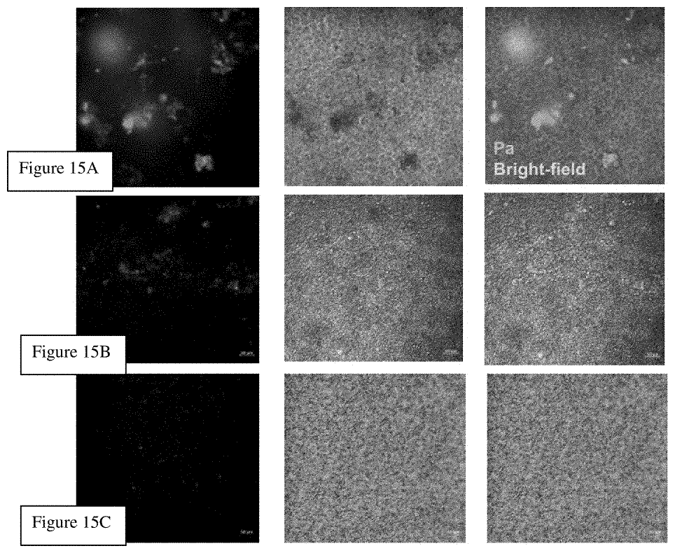

FIGS. 13A-C shows exemplary Imipenem (Merck compound) effects on P. aeruginosa infection, WT vs. mutant, on airway cells in Transwells. FIG. 13A shows exemplary Imipenem treatment. FIG. 13B shows exemplary Carbenicillin treatment. FIG. 13C shows exemplary Tetracycline treatment. Two-way ANOVA with Dunnett's post-test **<0.05, **<0.001, ***<0.0001 (compared to untreated).

FIGS. 14A-C shows exemplary Real time imaging of Imipenem effects on P. aeruginosa infection on one embodiment of a PA 5919 WT Pseudomonas infection on chip. FIG. 14A untreated (noninfected) control. FIG. 14B 50 μg/ml. FIG. 14C 500 μg/ml. PA 5919 WT 24 hpi. Left: Alexa fluor 488—anti-P. aeruginosa (Pa) antibody. Central: Bright-field. Right: Alexa fluor 488—anti-P. aeruginosa (Pa) antibody-Bright-field.

FIGS. 15A-C shows exemplary Real time imaging of Imipenem effects on P. aeruginosa infection on one embodiment of a PA 5890 Mutant 24 hpi Pseudomonas infection on chip. FIG. 15A untreated (noninfected) control. FIG. 15B 50 m/ml. FIG. 15C 500 μg/m. PA 5890 Mutant 24 hpi. Left: Alexa fluor 488—anti-P. aeruginosa (Pa) antibody. Central: Bright-field. Right: Alexa fluor 488—anti-P. aeruginosa (Pa) antibody-Bright-field.

H. Measuring Human β-Defensin 2 Secretion Post-Infection.

Prolonged P. aeruginosa infection results in enhancement of HBD-2 protein secretion.

Human beta-defensin 2 is measured in epithelial apical secretions using ELISA. Significance is **<0.05 according to an unpaired t-test.

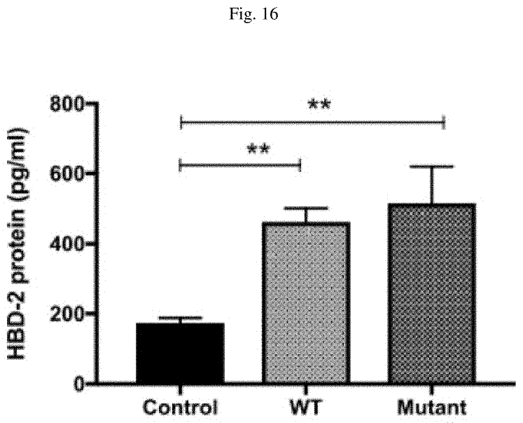

FIG. 16 shows an exemplary secretion of Human β-Defensin 2 post P. aeruginosa infection on one embodiment of a microfluidic Airway Chip. For comparison, HBD-2 protein (pg/ml) are measured in control chips; after WT P. aeruginosa; and a mutant strain of P. aeruginosa are tested for Human β-Defensin 2 secretion in apical wash, 24 hpi. Unpaired t-test, **<0.05. N=2.

I. Induction of Apoptosis in Host Cells post P. aeruginosa Infection.

One embodiment of an Airway On-Chip is infected as described above for 2 hours and then treated 50 μg/ml tetracycline for 24 h (top and bottom channels under flow) for 24 h in order to limit bacterial replication (to avoid over-replication and cell lifting). Using this methodology, we performed longer infections on an airway chip and avoided cell death at 24 h. At 24 h, chips are fixed with 4% paraformaldehyde for 15 minutes and subsequently permeabilized with 0.25% triton in PBS. Apoptosis staining is performed following the Click-iT™ Plus TUNEL Assay for In Situ Apoptosis Detection, Alexa Fluor 647 assay: Click-iT™ Plus TUNEL Assay, Thermo Fisher Scientific Inc., 6 November 2017.

The TUNEL assay is based on the incorporation of modified dUTPs by the enzyme terminal deoxynucleotidyl transferase (TdT) at the 3′-OH ends of fragmented DNA, a hallmark as well as the ultimate determinate of apoptosis. Click-iT™ Plus TUNEL assay with the Alexa Fluor™ 647 dye is utilized to detect the fragment DNA (purple), nuclei are labelled with DAPI. Uninfected cells show very limited apoptosis, whereas P. aeruginosa infected chips exhibited a significant increase in the number of apoptotic cells.

Staurosporin, a prototypical ATP-competitive kinase inhibitor, is used as a positive control in this assay. Chips incubated with 3 μM staurosporin for 3 h at 37° C. had increased apoptotic cell numbers. As an additional positive control, chips are treated with 1 unit of DNAse I for 30 minutes at room temperature to induce TUNEL positive DNA strand breaks. As shown in the figure, almost all DNAse I treated cells underwent apoptosis post treatment.

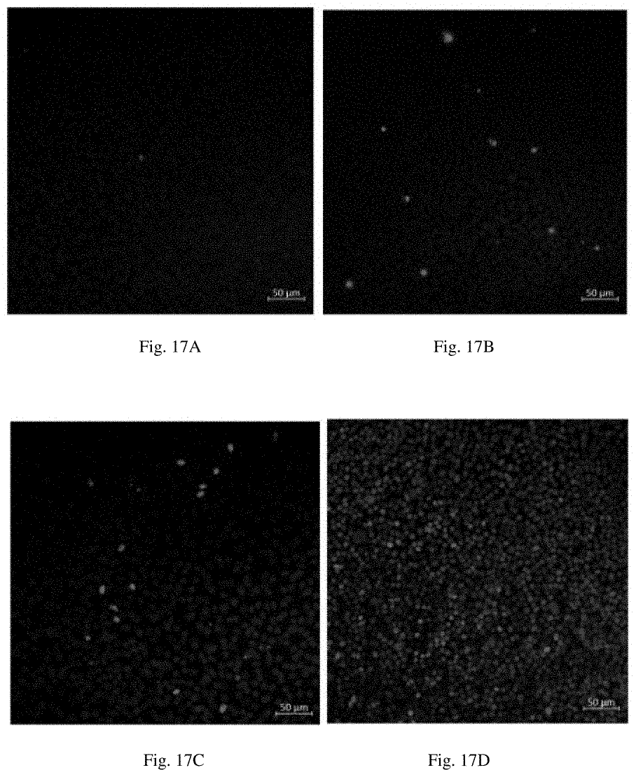

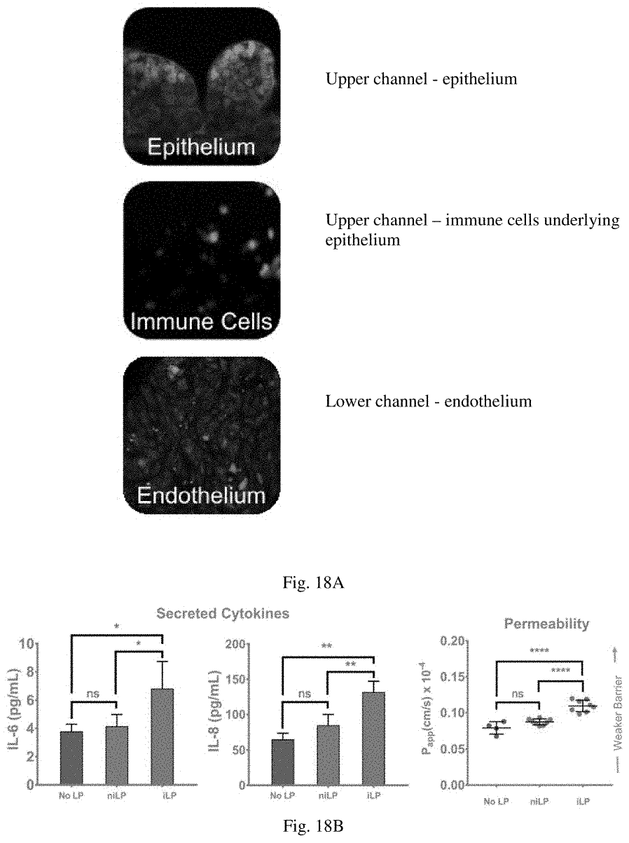

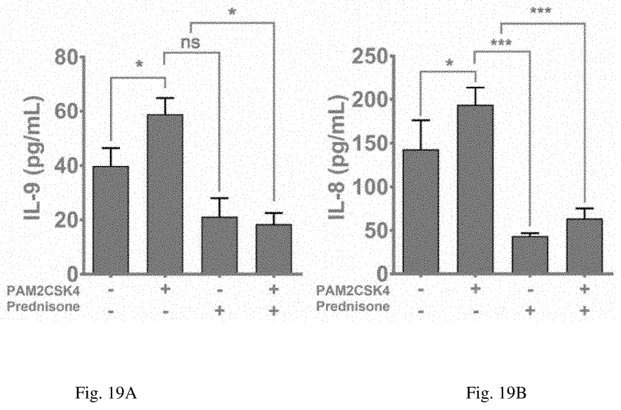

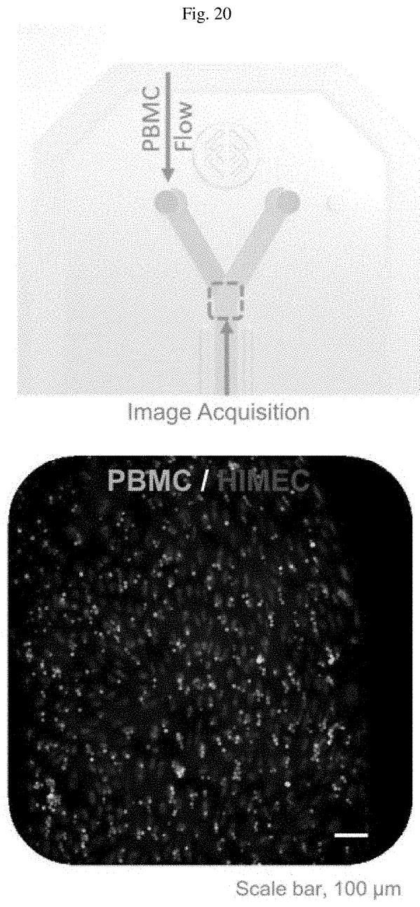

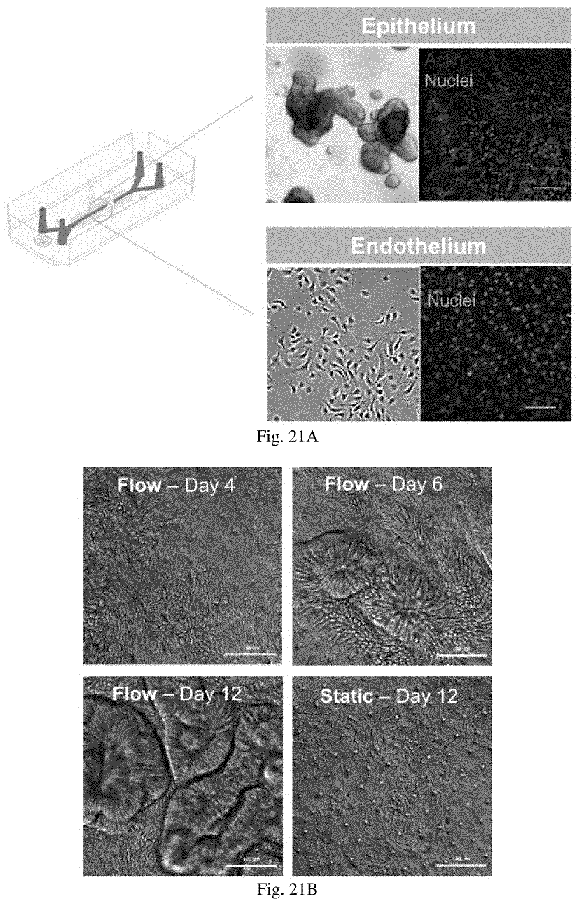

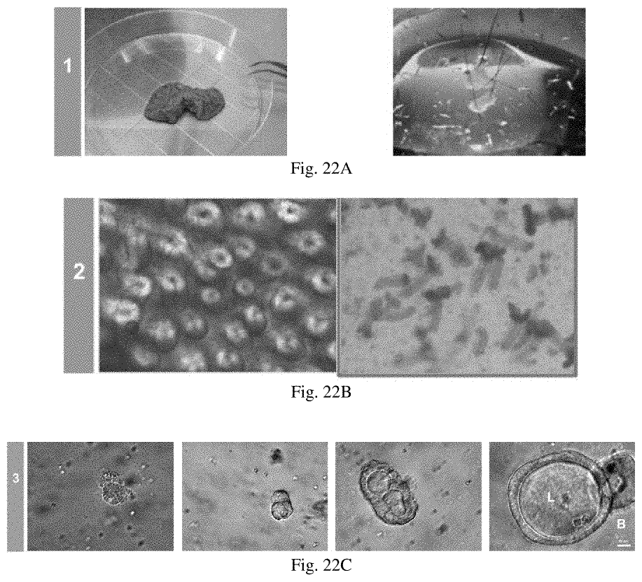

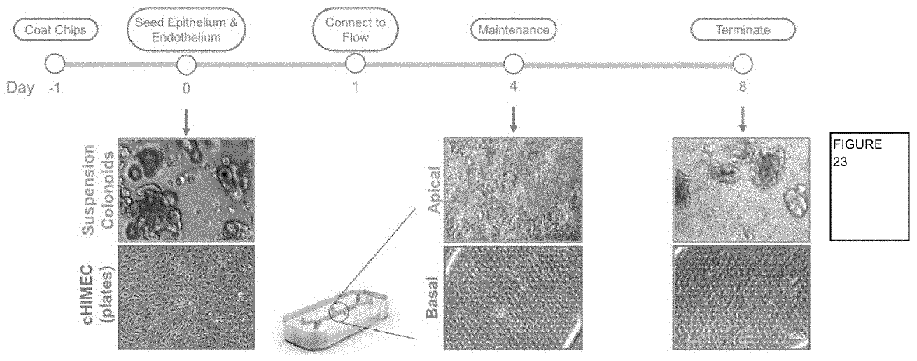

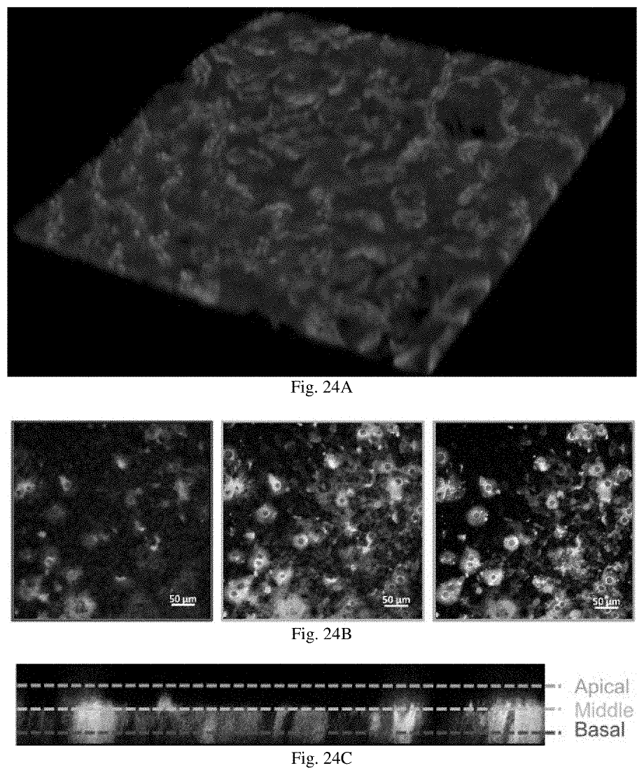

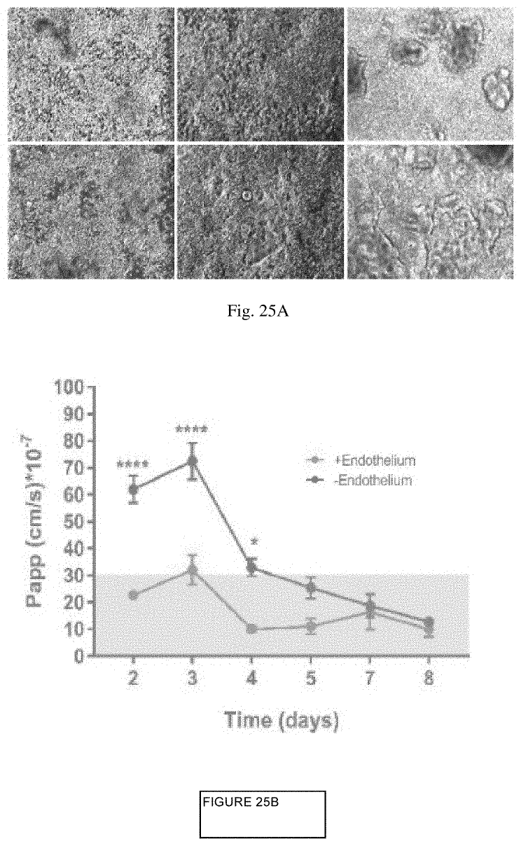

FIGS. 17A-D shows exemplary apoptosis via TUNEL staining at 24 h post infection. Apoptotic, TUNEL+, (pink); nuclei, DAPI+, (blue). FIG. 17A uninfected; FIG. 17B Pa infected; FIG. 57C staurosporin treatment. Staurosporin refers to an ATP-competitive kinase inhibitor. FIG. 57D DNAse I treatment. DNAse I refers to an endonuclease that nonspecifically cleaves DNA to release di-, tri- and oligonucleotide products with 5′-phosphorylated and 3′-hydroxylated ends. DNase I acts on single- and double-stranded DNA, chromatin and RNA:DNA hybrids.

Furthermore, readouts for an infected microfluidic chip, in particular for embodiments of bacterial infection of small airway chips, include but are not limited to: Real time imaging; Mucociliary activity; Immunofluorescence; Bacterial adherence; Drug effects; etc. Mucociliary activity includes but is not limited to automated, high-throughput ciliary beat analysis further including but not limited to beat frequency analysis flow velocity, direction, beat polarity and ciliary coverage, as a tool for chip quality control and diagnostics. Even further, readouts for bacterial infection of small airway chip in the context of disease models (such as CF and COPD), include but are not limited to: 1) Colonization and time-course infection with multiple pathogens (e.g. Moraxella catarrhalis, Pseudomonas aeruginosa, Streptococcus pneumonia, etc.); 2) Bacterial adherence to epithelial surface and quantification of % adherence and how this may differ between different bacterial strains in terms of colony forming units (CFUs); 3) Biofilm formation on airway epithelia; 4) Intracellular localization and intracellular survival of bacteria over time in epithelia (microscopy, CFU counting, etc.); 5) Effect of bacterial infection on ciliary beating frequency; 6) Effects of antimicrobial small compounds on bacterial killing and resolution of infection during airway infection; 7) Induction of antimicrobial peptide secretion in airway epithelia during infection, etc.

Additional readouts include but are not limited to cell function and gene expression. Although many of the readouts are described in association with microbial infected chips, readouts described herein may be used on any embodiment of a microfluidic intestine on-chip, including but not limited to embodiments of chips intended for base-line measurements, etc. In other words, any readout described herein may be used for evaluating any chip described herein.

Recreating the Cellular Microenvironment in Intestine On-Chips.

Microfluidic Intestine On-Chips have advantages over other types of culture devices including but not limited to having in vivo-like: extracellular matrix and cell interactions; cell shape and cytoarchitecture; tissue-tissue interactions; mechanical forces and a dynamic system, e.g. under fluid flow, providing shear forces/motility, stretching/peristalsis; control of luminal and vascular compositions, cellular, ECM, microbes, etc.; resident or circulating (e.g. peripheral) immune cells; microbiome interactions, e.g. co-culture of intestinal cells with microbes, including but not limited to commensal, pathogenic microbes; etc.

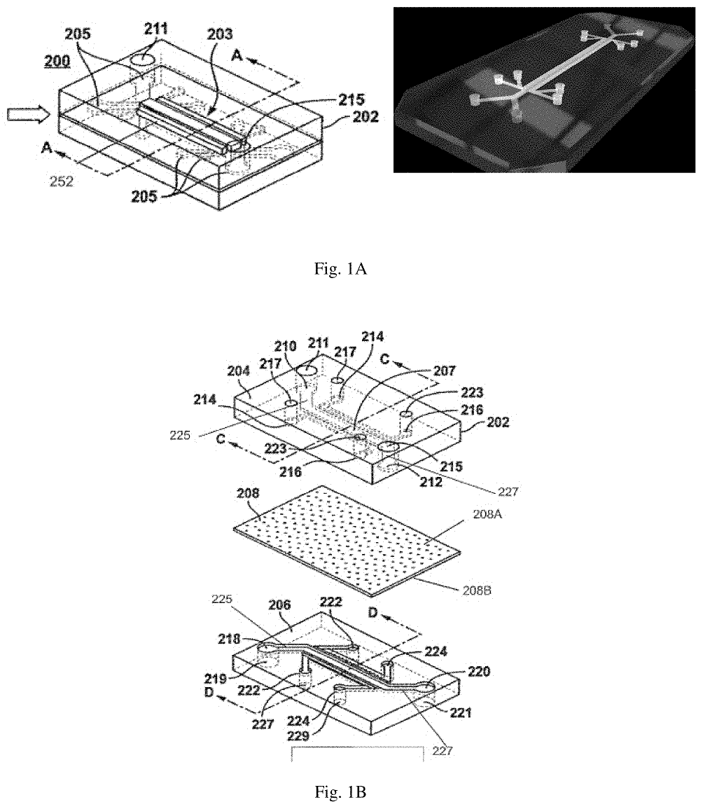

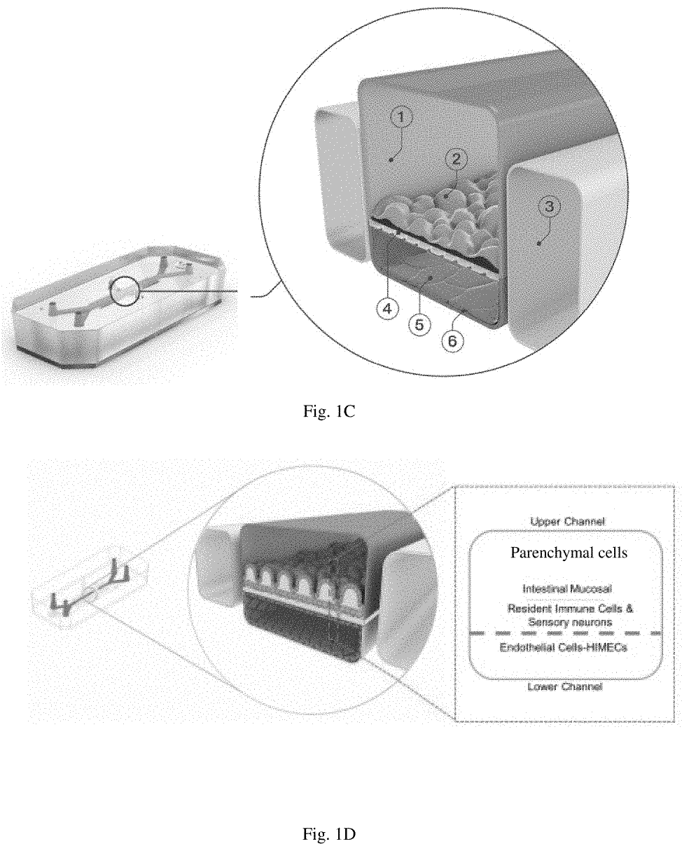

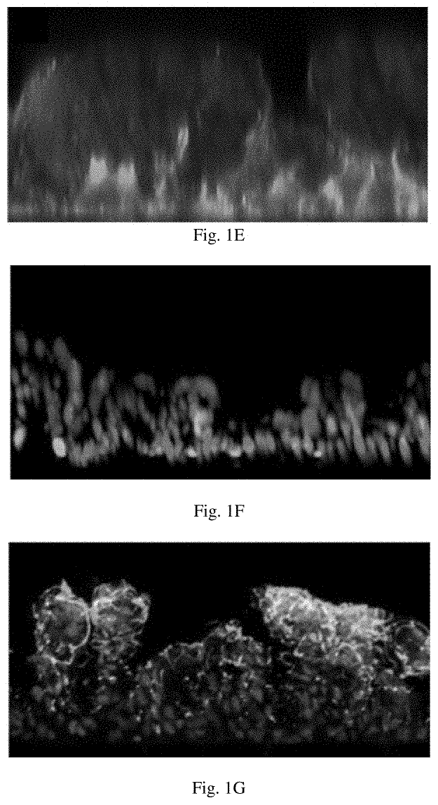

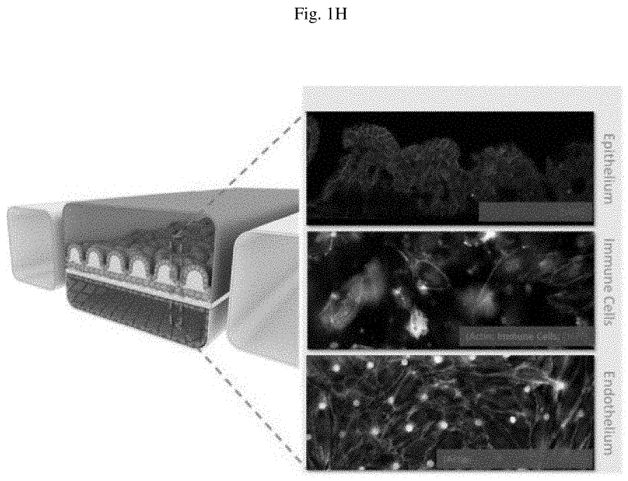

Exemplary embodiments of microfluidic intestine on-chip are shown in FIG. 1A-H.

FIG. 1A illustrates a perspective view of a microfluidic device with microfluidic channels in accordance with an embodiment (left) with a mirror image CAD illustration (right).

FIG. 1B illustrates an exploded view of a device in accordance with an embodiment, showing a microfluidic channel in a top piece and a microfluidic channel in a bottom piece, separated by a membrane.

FIG. 1C illustrates an exemplary schematic representation for one embodiment of a microfluidic intestine-on-chip: 1. Epithelial Channel; 2. Human Intestinal Epithelial Cells, e.g. Caco2, primary intestinal cells, cancer cells, etc.; 3. Vacuum Channel; 4. Membrane; 5. Human Intestinal Endothelial Cells e.g., HIMEC or iHIMEC, etc.; and 6. Vascular Channel. Designing and engineering the microenvironment allows us to recreate a “home away from home” for the cells within our Organ-Chips, including: Extracellular matrix and cell interactions; Cell shape and cytoarchitecture; Tissue-tissue interactions; Mechanical forces Dynamic system, including under flow and stretch, resident or circulating immune cells, etc.

FIG. 1D shows one embodiment of a microfluidic intestine-on-chip. In one embodiment a chip is comprised of at least two micro-channels (blue and pink channels) separated by a porous flexible-membrane. The material is functionalized with extracellular matrix and different types of cells are seeded into the two different channels. In one embodiment, of an Intestine On-Chip model, human endothelial cells are seeded in the bottom compartment (pink) and human epithelial cells in the top compartment (blue) to emulate the basic functioning unit of at least a portion of the intestine. Vacuum pressure can be applied to the side channels (gray) to mechanically stretch the membrane. Fluids can be continuously pumped through the channels to mimic shear forces, bring in nutrients, bring in bacteria, bring in test compounds and antibiotics, flush away wastes and provide effluent fluids for sampling. In another embodiment, of an Intestine On-Chip, resident immune cells are incorporated. In some embodiments, additional cells such as immune cells may be added. Environmental and Host Factors Implicated in GI Pathogenesis. Embodiments include additional cells such as intestinal mucosally derived cells or cultured cells such as found in the intestinal mucosa; resident immune cells, and sensory neurons in the epithelial channel.

FIGS. 1E-G shows immunofluorescent micrographs of immunostained embodiments of intestine on-chip derived from exemplary cell sources: Incorporating Patient-Derived Lamina Propria Immune Cells on-Chip. FIG. 1E Caco-2 BBE stained with Phalloidin—Actin (green), MUC2—Mucin 2 (purple), and DAPI—stained Nuclei colored blue.

FIG. 1F shows exemplary fluorescent micrographs of Primary Enteroids Muc2—Goblet Cells (pink), Lysozyme—Paneth Cells (green), and DAPI stained Nuclei colored blue.

FIG. 1G shows exemplary fluorescent micrographs of iPSC Organoids ZO-1—Tight Junction (green), E-cadherin (blue), Cdx2 stained Nuclei colored red.

FIG. 1H shows exemplary fluorescent micrographs (right) of embodiments of an intestine-on-chip (left) incorporating Lamina Propria Derived Immune Cells on the Intestine-Chip. Upper channel showing red tight junctions and blue colored DAPI staining of nuclei in the epithelial channel. Immune cells CD45+ colored purple, actin colored green and blue colored DAPI staining of nuclei in cells in the upper channel located below the epithelial cell layer. Endotheialial cells showing actin in green, cell-cell junctions in red and blue colored DAPI staining of nuclei in the lower endothelial channel.

In some embodiments, a microfluidic intestine On-Chip is a model for identifying microbe strain specific affects on morphology and physiology of intestinal cells, e.g. injury or growth; for identifying biomarkers for specific types of host-pathogen interactions; investigating potential probiotic functions of bacteria under normal and disease conditions for use in advising or treating patients, e.g. clinical groups, personalized medicine; etc.

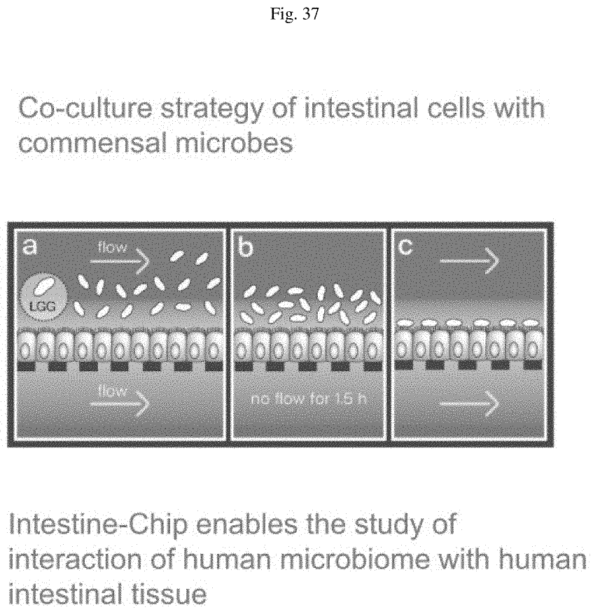

FIG. 37 illustrates embodiments comprising an exemplary Intestine On-Chip enabling microbiome studies. Co-culture strategy of intestinal cells with commensal microbes. Intestine-Chip enables the study of interaction of human microbiome with human intestinal tissue

Intestine On-Chip epithelial tissue sources include but are not limited to Caco-2 BBE cells; primary enteroids; iPSC Organoids, primary cells obtained from patient biopsies; etc. In one embodiment, an Intestine On-Chip is a small intestine on-chip. Cross-sectional representations of the different epithelial tissue sources available for the Intestine-Chip include but are not limited to: Caco-2 for rapid prototyping of new capabilities and patient-derived epithelium from both intestinal biopsy and iPSC.

As nonlimiting examples, FIG. 22A-C shows examples of intestinal cell sources for seeding onto microfluidic intestine on-chip devices as described herein.

FIG. 22A shows photographic images of one example of whole tissue obtained from a bowel (colon) resection, left, and an example of whole tissue obtained from a biopsy obtained by endoscopy, right.

FIG. 22B shows micrograph images of one example of dissociated cells at the beginning of culture, right, obtained (derived) from a biopsy of crypt tissue, left.

FIG. 22C shows an example of cells differentiated from stem cells as ex-vivo cultures in matrigel, starting from media comprising +Wnt3A, EGF, Noggin, R-spondin (WENR), which may be in cell culture dishes or on chip. Left to right, images show 3 day, 5 day, 7 day, and 10 day cultures. L-luminal, B-basal.

A. Establishing Intestine-Chips Generated From Patient Biopsies: Colon-Chip.

In one embodiment, an Intestine On-Chip is a colon intestine on-chip. The following are some exemplary embodiments describing and showing characteristics and features of a Colon (On)-Chip.

Biopsy derived enteroids offer tremendous advantages in part because of the differentiation of the major intestinal cell-types and the potential to study disease and drug responses in a patient-specific fashion. The capability to dissociate these monolayers on-Chip enables interrogation of the luminal and basolateral sides of the epithelium while controlling aspects of the microenvironment. As shown herein, a surface representation of the colonoid-derived epithelium with characteristic rugged morphology reminiscent of the colonic cytoarchitectures. F-actin staining reveals the in vivo-like rugged morphology of the colonoid derived epithelial monolayer on-Chip.

1. Colon-Chip Epithelial Layer Differentiation On-Chip: Polarization and Differentiation of the Colonic Epithelium.

In preferred embodiments, differentiation of colon tissue on-chip results in polarization and differentiation of the colonic epithelium on chip. Immunofluorescent staining of the epithelium reveals the presence of major colonic cell-types and contiguous network of tight junctional reflecting establishment of a tight epithelial barrier.

Characterizing healthy donor-donor variation of the differentiated cell-types is one of several readouts establishing a base-line for perturbations, such as one nonlimiting example, inclusion of other cellular components such as microvascular endothelial cells. Immunofluorescent staining of the epithelium from a donor reveals the presence of major colonic cell-types and contiguous network of tight junctional reflecting establishment of a tight epithelial barrier. Patient-specific, epithelial differentiation and formation of tight junctional networks are similar with and without the endothelial tissue interface.

Orthogonal views of the Colon-Chip epithelium show apical expression of both villin on absorptive enterocytes and MUC2 with luminal secretion of mucin into the fluidic channel. This indicates that on-Chip the epithelial monolayer has physiological cellular polarization and provides independent fluidic access to both the luminal and basolateral surfaces of the epithelium—an advantage over 3D, primary culture models.

FIG. 24A-C shows examples of immunostained images demonstrating polarization and differentiation of the colonic epithelium. Absorptive enterocytes and Goblet cells in the Colon-Chip show in vivo-like apical polarization. Thus, recreating Tissue-tissue interfaces dramatically accelerates epithelial barrier formation on the Colon-Chip.

FIG. 24A shows exemplary 3D reconstruction of one embodiment of an immunofluorescent image of a colon epithelium on-Chip. F-actin immunostaining shown in pink and nuclear DAPI staining shown in blue. Right panel is an enlarged image of one area of the right panel.

FIG. 24B shows exemplary images from left to right, basal plane, middle plane and apical plane matching the dotted line areas shown in a side-view in FIG. 24C. Villin (Absorptive)—green. Muc2 (Goblet)—pink. DAPI (Nuclei)—blue.

Epithelial Barrier Formation on the Colon-Chip. FIG. 25A-B shows examples of epithelial barrier formation on the Colon On-Chip. The presence of colon-specific microvascular cells improves the attachment of the epithelial tissue to the chip resulting in more robust monolayer with increased undulations characteristic of the rugged colonic morphology in-vivo. The endothelium also significantly improves the formation of epithelial barrier function on-Chip at early time-points indicating a key role in the maturation of absorptive enterocytes. FIG. 25A shows exemplary effects of endothelium upon barrier formation as bright field microscopic images. Upper row of bright filed images +(plus) endothelium in basal channel; lower row of images−(minus) endothelium, on Day 1, Day 4, and Day 8.

FIG. 25B shows measurements of exemplary apparent permeability (Papp(cm/s*10-7) of one embodiment of a colon-chip, with and without endothelium.

The intestinal barrier forms faster and appears tighter with endothelial cells cultured in the lower channel.

2. Differentiation of Absorptive Enterocytes and Goblet Cells and on a Colon-Chip: Presence of Endothelial Cells.

The presence of colon-specific microvascular cells improves the attachment of the epithelial tissue to the chip resulting in more robust monolayer with increased undulations characteristic of the rugged colonic morphology in-vivo. The endothelium also significantly improves the formation of epithelial barrier function on-Chip at early time-points indicating a role in the maturation of absorptive enterocytes.

Patient-specific epithelial differentiation is not significantly effected by the endothelial interface. One exemplary result in one embodiment of a microfluidic Intestine on-chip, goblet cell differentiation was not affected by the presence of endothelial cells. However, the presence of vasculature increases the expression of integrins, i.e. for one nonlimiting use, in contemplated heightened (increased) attachment to the extracellular matrix.

FIG. 27A-D shows exemplary colored immunofluorescent micrograph images of Colon-Chip Epithelial Differentiation of cells from one exemplary donor, immunostained for specified cell characteristics. Patient-specific sources of cells undergoing epithelial differentiation and formation of tight junctional networks are similar with and without the presence of endothelial cells, i.e. an endothelial tissue interface. FIG. 27A-D. FIG. 27A Villin (Absorptive) green and DAPI (Nuclei) blue; FIG. 27B ZO-1 (Tight Junctions) yellow and DAPI (Nuclei) blue; FIG. 27C ChrA (Enteroendocrine) turquoise and DAPI (Nuclei) blue; FIG. 27D MUC2 (Goblet) pink and DAPI (Nuclei).

3. Enteroendocrine Cell Localization on the Colon-Chip.

Immunofluorescence micrographs indicating the localization of enteroendocrine cells (EEC) within the differentiating Colon-Chip epithelium. In vivo-like tissue localization of chromogranin (ChrA) positive enteroendocrine cells. FIG. 28A-B Preliminary data suggests EEC cell differentiation may be depend on epithelial tissue height—reflecting in vivo-like crypt organization.

FIG. 28A-B shows exemplary immunofluorescent micrograph images of Enteroendocrine Cell Localization on the Colon-Chip. In vivo-like tissue localization of chromogranin positive, enteroendocrine cells. Preliminary data suggests EEC cell differentiation may be depend on epithelial tissue height—reflecting in vivo-like crypt organization.

FIG. 28A shows exemplary, left to right panels, DAPI Nuclei—blue; ZO-1 Tight Junctions—yellow; and ChrA (enteroenteroendocrine cells (EECs)-turquoise.

FIG. 28B shows exemplary merged images (FIG. 28A) with an area outlined by dotted lines (left panel) enlarged in the right panel.

4. Goblet Cell Differentiation on the Colon-Chip.

Goblet cells are responsible for the production of mucin and the formation of two thick mucus layers, the inner and the outer. The inner mucus layer consists of a firm layer of stratified mucin whereas the outer, which is approximately two times thicker than the inner, is made up of a loose network of mucins. The outer mucus layer is the microhabitat of the commensal bacteria. Under physiological conditions, commensal bacteria do not cross the sharp border line of the two layers and do not contact the epithelial barrier. In case of a homeostatic imbalance though, where the mucus composition and secretion are affected, the thickness of the mucus layer decreases, bringing the bacteria closer to the epithelium and facilitating the initiation of an immune reaction'.

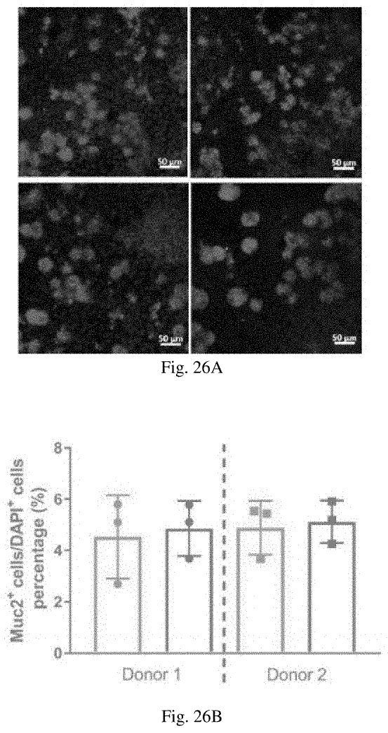

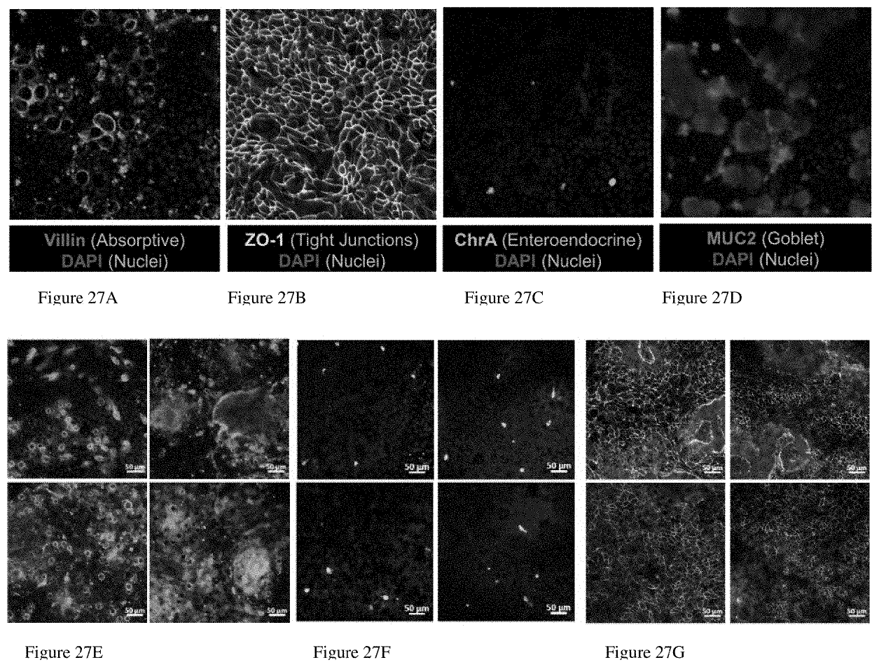

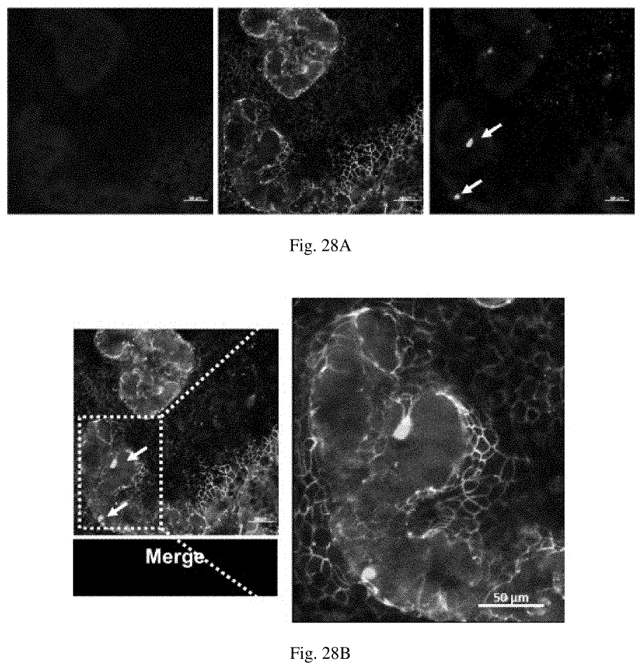

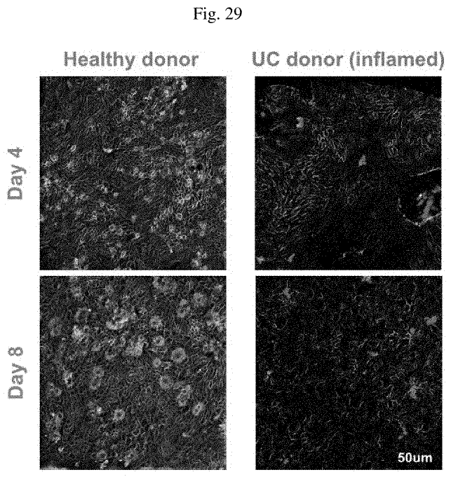

Exemplary immunostained goblet cells within a colon-chip epithelial layer differentiation of cells are shown from additional exemplary donors, see FIG. 26 A-B.

FIG. 26A-B shows exemplary microenvironment characteristics of one embodiment of a microfluidic Colon On-Chip: Goblet Cells grow in abundance as shown in intestinal cells derived (obtained) from at least 2 different donors.

FIG. 26A comparison of two different exemplary donors: Immunostained Donor 1, left panels; Donor 2, right panels. Muc2 (Goblet cells) colored pink, DAPI (Nuclei) colored blue.−(without) HIMEC upper row; +(with) HIMEC lower row.

FIG. 26B Comparison of goblet cell abundance in intestinal cells cultured from two different exemplary donors as goblet cell populations: Muc2+ cells/DAPI+ cells as a percentage (%) showing plus HIMEC (left blue bars) and without (−) HIMEC (right-grey bars).

Additionally, results using cells obtained (derived from) patient biopsies from UC inflamed donor also show the presence of goblet cells.

FIG. 29 shows exemplary goblet cells immunostained then colored pink on Day 4 vs. Day 8. Muc2+ pink (Goblet cells) Healthy donor (left) and UC donor (inflamed), right. E-cadherin+ (Tight Junctions)−yellow and Hoechst stain (Nuclei)−blue. Intestine On-Chip seeded with intestinal cells obtained from a patient with ulcerative colitis (UC) as diseased (right panel) compared to goblet cells in normal (left panel). A clear difference in goblet cell population is observed between the epithelium derived from healthy and UC diseased patients.

However, in one embodiment of a Colon-chip as described herein having colonic epithelium and tissue specific endothelial cells on opposing sides of a membrane, constantly exposed to laminar flow in both apical and basal sides, the abundance of Goblet cells is not relevant to those observed in vivo within healthy human colonic tissue. Goblet cells are responsible for the production of mucin so lower amounts of such cells would provide a human colonic epithelium in vitro with a lower amount of mucin than found in vivo. During the timeframe of 10 days during which the colonic epithelial cells are being differentiated into the three major colonic epithelial subtypes, absorptive enterocytes, goblet cells and enteroendocrine cells, an air-liquid interface was tested because in vivo there is periodical exposure to air and food chyme.

Surprisingly, by alternating exposure of the colonic epithelium to culture medium and air, which simulates their periodical exposure to the food chyme, resulted in duplication of the percentage of the Goblet cells found in vivo.

An Air-Liquid Interface (ALI) was extensively used in the differentiation of primary airway epithelial cells cultures^{1,2}, allowing and enhancing the formation of a pseudostratified monolayer, consisted of basal, ciliated and secretory/goblet cells. See also, exemplary microfluidic airway-on-chips, described herein.

In regards to the intestinal experimental models ALI was applied in organotypic³⁻⁷ and ex vivo tissue cultures⁸, aiming for constant oxygenation and enhanced viability of the epithelial monolayer. Specifically, in vitro experiments executed on porcine cells, ALI resulted in a stable oxygenation, increased oxidative phosphorylation and differentiation of the intestinal epithelium^{3,7}.

Oxygen depletion, also referred as hypoxia, was linked with the activation of Notch pathway in cancer models⁹. Notch pathway was also linked to the multineage differentiation of the Intestinal Stem Cells (ISCs), whereas its inhibition commits epithelial progenitor cells in the secretory phenotype¹⁰. Based on the aforementioned literature and taking into account that Goblet cells are the most abundant secretory cells in the distal GI tract, we chose to test induction of periodical ALI then assess its effect on primary colonic epithelial cell differentiation.

Therefore, we modified one embodiment of a method for providing a Colon-Chip, described below, by introducing air in the apical channel following the completion of 24 hrs culture in the presence of cycling stretching, then maintained a static ALI for 6 hrs with the apical channel immersed in culture medium for 18 hrs. This cyclic culture, therefore called “Periodical ALI” or “Cyclic ALI”, was repeated until the last day of the culture. Characterization of the epithelial cells, using immunofluorescence, revealed duplication of the Goblet cells population in vitro, as observed in vivo, in the presence of “Periodical ALI” in comparison to the constant LLI (Liquid-Liquid Interface) culture (3.55±0.39-LLI VS 6.22±0.71-Periodical ALI, Mean±SD, n=1 experiment). The same increasing trend was detected in the Muc2 mRNA expression on a repetition of this experiment. (FIG. 82A-C).

Thus, we used the method described above for optimizing the colon chip model described herein for enhancing Goblet cells differentiation for achieving the formation of a physiological relevant mucus layer. Therefore, in some embodiments, Colon-Chip co-cultures with living microbes further use a Periodical ALI for a more physiologically relevant model of microbial interactions.

Additional benefits of using the Periodical ALI during expansion and differentiation of primary derived human colonic epithelial cells into a monolayer include, but are not limited to: using media without removing the maintenance of sternness, factors (i.e. Wnt3a-EGF-Rspondinl-Noggin, WENR); recapitulating the periodicity of the food chyme transition of intestinal cell contact within the gastrointestinal tract; etc.

Exemplary preparation of a Colon-chip includes but is not limited to the following method.

Activated S1 chips (or tall channel chips) using 0.5 mg/ml Sulfo-SANPAH under a UV light lamp. Channels are then washed out with 10 Mm HEPES solution and PBS1X. Each channel is subsequently submerged into a different coating solution and incubated overnight at 37° C. Apical channel is coated with 200 ug/ml Collagen IV (human placenta derived) and 100 ug/ml Matrigel, whereas basal channel with 200 ug/ml Collagen IV (human placenta derived) and 30 ug/ml Fibronectin (human).

After the ECM coating solution is removed via PBS1X washes, endothelial cells are introduced in the basal channel and cultured in the presence of a commercially available medium EGM2-MV (Vendor: Promocell). Endothelial cells seeding lasts 30 min-1 hr and at 37oC with the chips inverted so that the endothelial cells will attach to the membrane. Following, primary derived colonoids, being recovered from the Matrigel based suspension culture, are briefly digested (e.g. tyrpsin, sonication) to break up clumps then inserted as fragments of clumps in the apical channel and cultured in the presence of the commercially available medium IntestiCult (Vendor: IntestiCult). For the first two days of culture (Day 0-2) epithelial cells are maintained in the presence of the stemness supporting factors, Y27632 (ROCK inhibitor) and CH1R99021 (Wnt agonist).

Day 1: The seeded chips are connected to the PODs and laminar flow of 60 ul/hr is applied on both channels.; Day 2: CHIR99021 and Y-27632 are removed from the apical channel medium.; Day 3: Cells are being acclimated to cycling stretching by 2%, 0.15 Hz stretching application.; Day 4: Stretching is increased to 10%, 0.15 Hz and maintained at this level until the end of the fluidic culture (Day 10).

This protocol was used for the expansion of human colonoids in a monolayer in a microfluidic culture for at least three different healthy tissue donors. Moreover, we discovered that the colonic stem and progenitor cells (present and abundant in the conventional suspension culture in Matrigel) are able to differentiate into the three basic epithelial cell subtypes, Goblet cells (FIG. 81A-C), absorptive enterocytes and enteroendocrine cells without the removal of the necessary stemness factors (Wnt3a-EGF-Rspondin1-Noggin, WENR) as described in previous protocols^{11,12,13}.

The achieved percentage of the Goblet cells though (2.54±0.95, Mean±SD calculated across three donors) was well below the physiologically found in normal adult colon (6-15% across the proximodistal axis of the GI tract¹⁴).

The following references related to Section 3 above, are herein incorporated in their entirety:
1. Nichols, J. E. et al. Modeling the lung: Design and development of tissue engineered macro- and micro-physiologic lung models for research use. Exp. Biol. Med. (2014). doi:10.1177/1535370214536679
2. Schilders, K. A. A. et al. Regeneration of the lung: Lung stem cells and the development of lung mimicking devices. Respiratory Research (2016). doi:10.1186/S12931-016-0358-Z
3. Nossol, C. et al. Air-liquid interface cultures enhance the oxygen supply and trigger the structural and functional differentiation of intestinal porcine epithelial cells (IPEC). Histochem. Cell Biol. (2011). doi:10.1007/s00418-011-0826-y
4. Viney, M. E., Bullock, A. J., Day, M. J. & MacNeil, S. Co-culture of intestinal epithelial and stromal cells in 3D collagen-based environments. Regen. Med (2009). doi:10.2217/rme.09.4
5. Dimarco, R. L. et al. Engineering of three-dimensional microenvironments to promote contractile behavior in primary intestinal organoids. Integr. Biol. (United Kingdom) (2014). doi:10.1039/c3ib40188j
6. Ootani, A. et al. Sustained in vitro intestinal epithelial culture within a Wnt-dependent stem cell niche. Nat. Med. (2009). doi:10.1038/nm.1951
7. Klasvogt, S. et al. Air-liquid interface enhances oxidative phosphorylation in intestinal epithelial cell line IPEC-J2. Cell Death Discov. (2017). doi:10.1038/cddiscovery.2017.1
8. Grivel, J. C. & Margolis, L. Use of human tissue explants to study human infectious agents. Nat. Protoc. (2009). doi:10.1038/nprot.2008.245
9. Pear, W. S. & Simon, M. C. Lasting longer without oxygen: The influence of hypoxia on Notch signaling. Cancer Cell (2005). doi:10.1016/j.ccr.2005.11.016
10. Pellegrinet, L. et al. DII1—and DII4-mediated notch signaling are required for homeostasis of intestinal stem cells. Gastroenterology (2011). doi:10.1053/j.gastro.2011.01.005
11. Kasendra, M. et al. Development of a primary human Small Intestine-on-a-Chip using biopsy-derived organoids. Sci. Rep. (2018). doi:10.1038/s41598-018-21201-7
12. Kozuka, K. et al. Development and Characterization of a Human and Mouse Intestinal Epithelial Cell Monolayer Platform. Stein Cell Reports (2017). doi:10.1016/j.stemcr.2017.10.013
13. Yin, J. et al. Molecular Basis and Differentiation-Associated Alterations of Anion Secretion in Human Duodenal Enteroid Monolayers. CMGH (2018). doi:10.1016/j.jcmgh.2018.02.002
14. Karam, S. M. LINEAGE COMMITMENT AND MATURATION OF EPITHELIAL CELLS IN THE GUT. Frontiers in Bioscience (1999). doi:10.2741/Karam
15. Johansson, M. E. V., Larsson, J. M. H. & Hansson, G. C. The two mucus layers of colon are organized by the MUC2 mucin, whereas the outer layer is a legislator of host-microbial interactions. Proc. Natl. Acad. Sci. (2011). doi:10.1073/pnas.1006451107

B. Modeling Oxygen Microenvironments in the Intestine-Chip.

Organ-Chips were developed to have controlled oxygen microgradients supporting epithelial cell differentiation and function, in addition to supporting physiological compositions of luminal microbes in a tissue-specific fashion. Therefore, at least one tissue-matched physoxia condition was established enabling the support of complex microbial communities on-chip.

A regulatory effect of hypoxia on tissue homoestasis is evident because of increased proliferation a well as elevated apoptosis at the crest of the epithelial undulations.

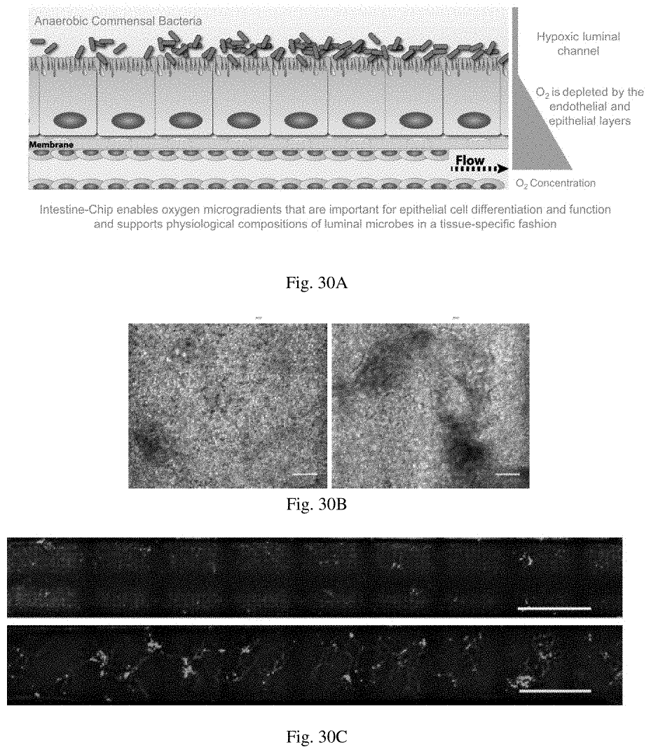

FIG. 30B-C demonstrates an exemplary effect of physoxia on Colon On-Chip cellular morphology.

FIG. 30B shows exemplary bright-field micrographs of cells on chips under 21% O₂ (left) vs. 5% O₂ (right).

FIG. 30C shows exemplary apoptosis of cells compared between 2 chips, were apoptotic cells are colored green (TUNEL+)/DAPI (blue) under 21% O₂ (upper) vs. 5% O₂(lower). Physiological oxygen concentrations (physoxia), around 5% O₂, improves Colon-Chip tissue morphology and increases the rate of cellular apoptosis, a feature of healthy epithelial homeostasis.

Providing phyoxia conditions in one embodiment of a microfluidic Intestine on-Chip enables co-culture with microbial species representing the facultative and fastidious anaerobes found in colonic communities that are regulators of intestinal health and disease.

Effects of Physoxia on Colon-Chip Morphology: Modeling Oxygen Microenvironments in the Intestine-Chip.

Organ-Chips enables oxygen microgradients supporting epithelial cell differentiation and function, in addition to supporting physiological compositions of luminal microbes in a tissue-specific fashion.

In one embodiment, anaerobic commensal bacteria are co-cultured with intestinal epithelial cells in an upper channel, with endothelial cells lining a lower channel under fluid flow. The O₂ concentration is higher in the lower channel as media provides O₂, then as the endothelial cells consume O₂ and O₂ diffuses into the epithelial layers where it is consumed by cells in the epithelium, O₂ is depleted in the apical regions providing a hypoxic luminal channel where microbes, e.g. bacteria, are added to fluid bathing apical regions.

FIG. 30A illustrates physiological oxygen concentrations improves Colon-Chip tissue morphology and increases the rate of apoptosis, a feature of epithelial homeostasis.

FIG. 30A illustrates an exemplary hypoxic luminal channel where O₂ from the lower channel is depleted by the endothelial cells and basal end of the epithelial cells so that the upper apical region of the epithelial layer is hypoxic where anaerobic bacteria may grow. O₂ Concentration is represented on the right side in blue, where the wider blue area represents a more oxygenated area than the upper thinner blue area.

FIG. 30B-C demonstrates an exemplary effect of physoxia on Colon On-Chip cellular morphology.

FIG. 30B shows exemplary bright-field micrographs of cells on chips under 21% O₂ (left) vs. 5% O₂ (right).

FIG. 30C shows exemplary apoptosis of cells compared between 2 chips, were apoptotic cells are colored green (TUNEL+)/DAPI (blue) under 21% O₂ (upper) vs. 5% O₂ (lower). Physiological oxygen concentrations (physoxia), around 5% O₂, improves Colon-Chip tissue morphology and increases the rate of cellular apoptosis, a feature of healthy epithelial homeostasis.

Co-Culture with Commensal Bacterial Strains on Colon-Chip.

Microbial species representing the facultative and fastidious anaerobes found in colonic communities that are regulators of intestinal health and disease.

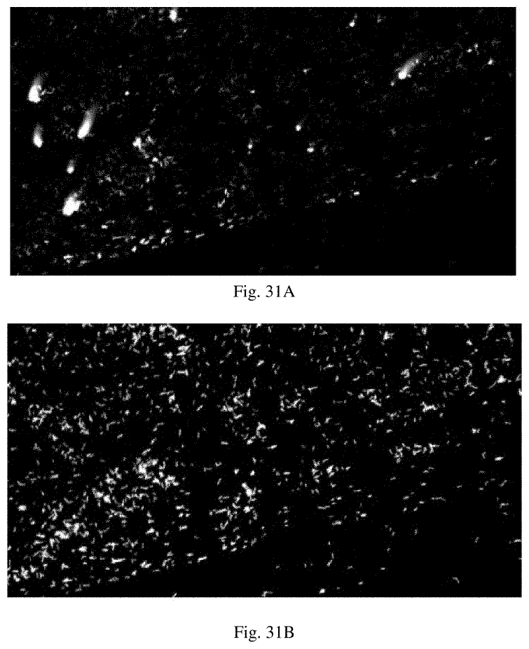

FIG. 31A-B shows exemplary commensal bacterial strains; Lactobacillus rhamnosus GG (LGG) and Clostridia (C.) symbiosum on a microscope slide. Samples were obtained from inoculum before mixing the populations 1:1 and introducing them into one embodiment of a Colon-Chip. Physoxia microenvironment supports Colon On-Chip epithelium.

FIG. 31A shows exemplary LGG bacteria (white).

FIG. 31B shows exemplary C. symbiosum bacteria (white).

Morphology of Colon-Chip in Normal and Physoxia Microenvironment. The Colon-Chip epithelium in physoxia conditions maintains a healthy morphology with no observable differences in the presence of co-culture with C. symbiosum. Thus, physoxia microenvironments support a healthy Colon-Chip epithelium and co-culture with anaerobic microbes.

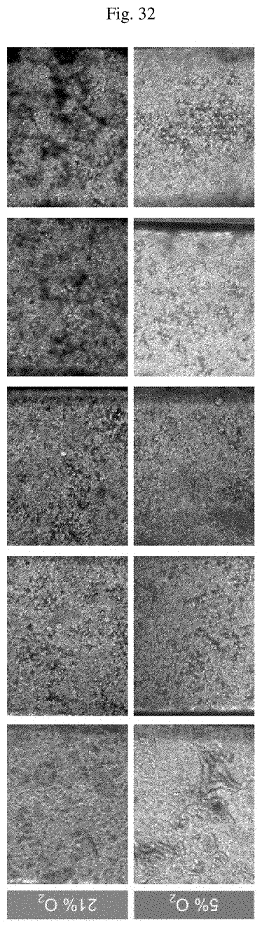

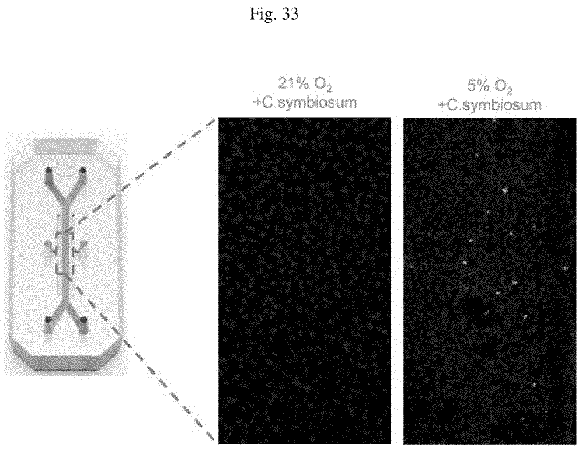

FIG. 32 shows exemplary morphology of Colon-Chip in normal (21% O₂) vs. physoxia microenvironment (5% O₂). 21% O₂ (upper row showing micrographs of cells) vs. 5% O₂ (lower row showing micrographs of cells) over time, left to right, Day 0, Day 3, Day 4, Day 7 and Day 7 plus C. symbiosum. FIG. 33 shows an exemplary embodiment of a Colon On-Chip co-culture with anaerobic commensal bacteria. A physoxia microenvironment (5% Oxygen) on the Colon-Chip supports anaerobic commensal bacteria. Left image shows an illustration of a micofluidic device, florescent micrographs show immunostained C. symbiosum (green) cells obtained from the central upper channel (dotted lines) comparing chips having 21% O₂+C. symbiosum (middle panel) vs. 5% O₂ C. symbiosum (right). DAPI stained nuclei of epithelial cells shown in blue.

Colon-Chip Co-Culture with Anaerobic Commensal Bacteria.

Physoxia microenvironment on the Colon-Chip supports anaerobic commensal bacteria. Co-culture with pre-labeled C. symbiosum (green) and LGG for 48 hours on the Colon-chip shows the presence of viable anaerobic bacteria in the physoxia condition relative to the normoxia conditions where no C. symbiosum were evident.

FIG. 33 shows an exemplary embodiment of a Colon On-Chip co-culture with anaerobic commensal bacteria. A physoxia microenvironment (5% Oxygen) on the Colon-Chip supports anaerobic commensal bacteria. Left image shows an illustration of a micofluidic device, florescent micrographs show immunostained C. symbiosum (green) cells obtained from the central upper channel (dotted lines) comparing chips having 21% O₂+C. symbiosum (middle panel) vs. 5% O₂ C. symbiosum (right). DAPI stained nuclei of epithelial cells shown in blue.

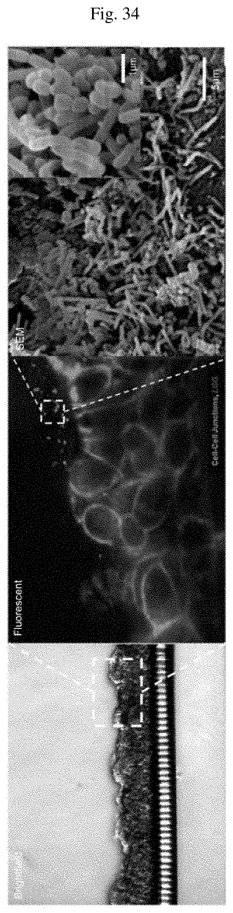

FIG. 34 shows an exemplary epithelial interaction with LGG. Left to right, a Brightfield image of a cross-section of one embodiment of a microfluidic intestine on-chip showing cells growing on-chip; middle shows a fluorescent micrograph of immunostained cell-cell junctions (green) and immunostained bacteria (purple) representing the area outlined in white dotted line (left); right paned shows a Scanning electron micrograph (SEM) of bacteria attached to epithelial cells (upper right enlarged micrograph showing individual bacterium). Thus, one embodiment of an Intestine On-Chip has epithelial microbiome interactions.

In summary, some embodiments of a Colonoid-derived Intestine-Chip provide a recapitulation of physiological microenvironments of colon tissue, including but not limited to tissue-tissue interfaces; physiological mechanical forces; oxygen microgradients; provides a differentiated monolayer of colonic epithelium with major cell-subtypes; enables mechanistic studies of host-microbe interactions; etc.

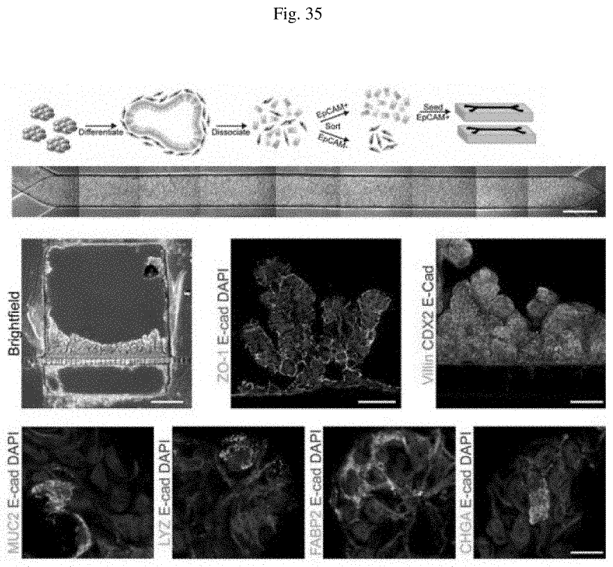

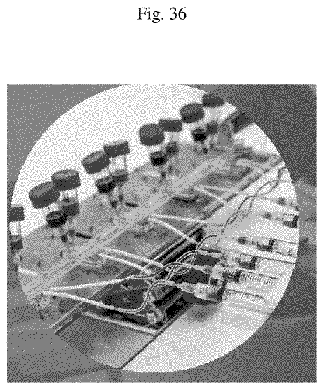

FIG. 35 shows an exemplary embodiment of an iPSC derived Intestine On-Chip. Epithelium derived from 3D intestinal organoids (illustrated in upper panel), as differentiating cells cultured in dishes then seeded onto chips or differentiated on chip then disassociated and used to seed a second chip. Differentiation of major intestinal cell-types in and maturation of a 3D villus-like morphology on-Chip. Applications include but are not limited to: Potential for differentiation of other patient-matched tissue cell-types particularly of immune lineage; and Applications in precision medicine and disease modeling.

FIG. 37 illustrates embodiments comprising an exemplary Intestine On-Chip enabling microbiome studies. Co-culture strategy of intestinal cells with commensal microbes. Intestine-Chip enables the study of interaction of human microbiome with human intestinal tissue.

### DETAILED DESCRIPTION OF THE INVENTION

### I. Microfluidic Chips, Devices and Systems.

Microfluidic chips, devices, and systems contemplated for use include but are not limited to chips described in Bhatia and Ingber, “Microfluidic organs-on-chips.” Nature Biotechnology, 32(8):760-722, 2014; U.S. Pat. No. 8,647,861, Organ mimic device with microchannels and methods of use and manufacturing thereof, herein incorporated in its entirety by reference, for some examples. Further, systems are used for loading (introducing) fluids, including but not limited to fluids containing nutrients, media, cytokines, bacteria, etc. In some embodiments, such systems are used for collecting effluent, in part for use in measuring cytokines.

The following section is merely for providing nonlimiting examples of embodiments that may find use as microfluidic devices.

### II. Closed Top Chips.

The present disclosure relates to gut-on-chips, such as fluidic devices comprising one or more cells types for the simulation one or more of the function of gastrointestinal tract components. Accordingly, the present disclosure additionally describes closed-top intestine-on-chips, see, e.g. schematic in FIG. 1A-B.

A. Closed Top Microfluidic Chips without Gels.

In one embodiment, closed top gut-on-chips do not contain gels, either as a bulk gel or a gel layer. Thus, in one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer.

Additional embodiments are described herein that may be incorporated into closed top chips without gels.

B. Closed Top Microfluidic Chips With Gels.

In one embodiment, closed top gut-on-chips do contain gels, such as a gel layer, or bulk gel, including but not limited to a gel matrix, hydrogel, etc. Thus, in one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. In some embodiments, the device further comprises a gel. In some embodiments, the gel is a continuous layer. In some embodiments, the gel is a layer of approximately the same thickness across the layer. In some embodiments, the gel is a discontinuous layer. In some embodiments, the gel has different thicknesses across the layer. In some embodiments, the first side of the membrane may have a gel layer. In some embodiments, a gel is added to the first side of the membrane without an ECM layer. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer. In some embodiments, the gel layer is above the ECM coating layer. In some embodiments, the ECM coating layer may have a gel layer on the bottom, i.e. the side facing the membrane. In some embodiments, the gel overlays the ECM gel layer.

Additional embodiments are described herein that may be incorporated into closed top chips with gels.

C. Closed Top Microfluidic Chips with Simulated Lumens.

A closed top gut-on-chip comprising a gel-lined simulated lumen may be used for generating a more physiological relevant model of gastrointestinal tissue. In some embodiments, closed top gut-on-chips further comprise a gel simulated three-dimensional (3-D) lumen. In other words, a 3-D lumen may be formed using gels by providing simulated intestinal villi (e.g. viscous fingers) and/or mimicking intestinal folds. In a preferred embodiment, the gel forms a lumen, i.e. by viscous fingering patterning.

Using viscous fingering techniques, e.g. viscous fingering patterning, a simulated intestinal lumen may be formed by numerous simulated intestinal villi structures. Intestinal villi (singular: villus) refer to small, finger-like projections that extend into the lumen of the small intestine. For example, healthy small intestine mucosa contains these small finger-like projections of tissue that are present along the lumen as folds of circular plica finger-like structures. A villus is lined on the luminal side by an epithelia cell layer, where the microvillus of the epithelial cells (enterocytes) faces the lumen (i.e. apical side). Viscous fingers may be long and broad, for mimicking villi in the duodenum of the small intestine, while thinner or shorter viscous fingers may be used for mimicking villi in other parts of the gastrointestinal tract. As one example, viscous fingers may be formed and used to mimic epithelial projections in the colon.

Methods to create three-dimensional (3-D) lumen structures in permeable matrices are known in the art. One example of a 3-D structure forming at least one lumen is referred to as “viscous fingering”. One example of viscous fingering methods that may be used to for form lumens, e.g. patterning lumens, is described by Bischel, et al. “A Practical Method for Patterning Lumens through ECM Hydrogels via Viscous Finger Patterning.” J Lab Autom. 2012 April; 17( 2): 96-103. Author manuscript; available in PMC 2012 Jul. 16, herein incorporated by reference in its entirety. In one example of a viscous finger patterning method for use with microfluidic gut-on-chips, lumen structures are patterned with an ECM hydrogel.

“Viscous” generally refers to a substance in between a liquid and a solid, i.e. having a thick consistency. A “viscosity” of a fluid refers to a measure of its resistance to gradual deformation by shear stress or tensile stress. For liquids, it corresponds to an informal concept of “thickness”; for example, honey has a much higher viscosity than water.

“Viscous fingering” refers in general to the formation of patterns in “a morphologically unstable interface between two fluids in a porous medium.

A “viscous finger” generally refers to the extension of one fluid into another fluid. Merely as an example, a flowable gel or partially solidified gel may be forced, by viscous fingering techniques, into another fluid, into another viscous fluid in order to form a viscous finger, i.e. simulated intestinal villus.

In some embodiments, the lumen can be formed by a process comprising (i) providing the first chamber filled with a viscous solution of the first matrix molecules; (ii) flowing at least one or more pressure-driven fluid(s) with low viscosity through the viscous solution to create one or more lumens each extending through the viscous solution; and (iii) gelling, polymerizing, and/or cross linking the viscous solution. Thus, one or a plurality of lumens each extending through the first permeable matrix can be created.

In another embodiment, gel is added to a channel for making a lumen.

In some embodiments as described herein, the first and second permeable matrices can each independently comprise a hydrogel, an extracellular matrix gel, a polymer matrix, a monomer gel that can polymerize, a peptide gel, or a combination of two or more thereof. In one embodiment, the first permeable matrix can comprise an extracellular matrix gel, (e.g. collagen). In one embodiment, the second permeable matrix can comprise an extracellular matrix gel and/or protein mixture gel representing an extracellular miroenvironment, (e.g. MATRIGEL®. In some embodiments, the first and second permeable matrixes can each independently comprise a polymer matrix. Methods to create a permeable polymer matrix are known in the art, including, e.g. but not limited to, particle leaching from suspensions in a polymer solution, solvent evaporation from a polymer solution, sold-liquid phase separation, liquid—liquid phase separation, etching of specific “block domains” in block co-polymers, phase separation to block-co-polymers, chemically cross-linked polymer networks with defined permabilities, and a combination of two or more thereof.

Another example for making branched structures using fluids with differing viscosities is described in “Method And System For Integrating Branched Structures In Materials” to Katrycz, Publication number US20160243738, herein incorporated by reference in its entirety.

Regardless of the type of lumen formed by a gel and/or structure, cells can be attached to theses structures either to lumen side of the gel and/or within the gel and/or on the side of the gel opposite the lumen. Thus, three-dimensional (3-D) lumen gel structures may be used in several types of embodiments for closed top microfluidic chips, e.g. epithelial cells can be attached to outside of the gel, or within the gel. In some embodiments, LPDCs may be added within the gel, or below the gel, on the opposite side of the lumen. In some embodiments, stoma cells are added within the gel. In some embodiments, stomal cells are attached to the side of the gel opposite from the lumen. In some embodiments, endothelial cells are located below the gel on the side opposite the lumen. In some embodiments, endothelial cells may be present within the gel.

Additional embodiments are described herein that may be incorporated into closed top chips with simulated 3D lumens containing a gel.

III. Open Top Microfluidic Chips.

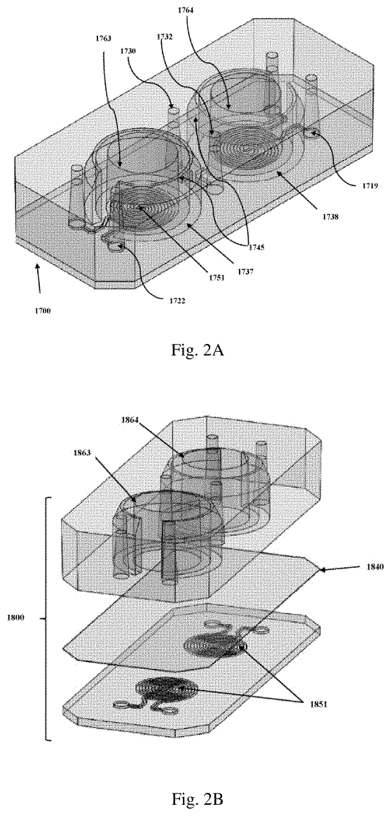

The present disclosure relates to gut-on-chips, such as fluidic devices comprising one or more cells types for the simulation one or more of the function of gastrointestinal tract components. Accordingly, the present disclosure additionally describes open-top gut-on-chips, see, e.g. schematic in FIG. 2A-B. U.S. Pat. No. 8,647,861, Organ mimic device with microchannels and methods of use and manufacturing thereof B shows an exemplary exploded view of one embodiment of an open-top chip device 1800, wherein a membrane 1840 resides between the bottom surface of the first chamber 1863 and the second chamber 1864 and the at least two spiral microchannels 1851. Open top microfluidic chips include but are not limited to chips having removable covers, such as removable plastic covers, paraffin covers, tape covers, etc.

Many of the problems associated with earlier systems can be solved by providing an open-top style microfluidic device that allows topical access to one or more parts of the device or cells that it comprises. For example, the microfluidic device can include a removable cover, that when removed, provides access to the cells of interest in the microfluidic device. In some aspects, the microfluidic devices include systems that constrain fluids, cells, or biological components to desired area(s). The improved systems provide for more versatile experimentation when using microfluidic devices, including improved application of treatments being tested, improved seeding of additional cells, and/or improved aerosol delivery for select tissue types.

It is also desirable in some aspects to provide access to regions of a cell-culture device. For example, it can be desirable to provide topical access to cells to (i) apply topical treatments with liquid, gaseous, solid, semi-solid, or aerosolized reagents, (ii) obtain samples and biopsies, or (iii) add additional cells or biological/chemical components

Therefore, the present disclosure relates to fluidic systems that include a fluidic device, such as a microfluidic device with an opening that provides direct access to device regions or components (e.g. access to the gel region, access to one or more cellular components, etc.). Although the present disclosure provides an embodiment wherein the opening is at the top of the device (referred to herein with the term “open top”), the present invention contemplates other embodiments where the opening is in another position on the device. For example, in one embodiment, the opening is on the bottom of the device. In another embodiment, the opening is on one or more of the sides of the device. In another embodiment, there is a combination of openings (e.g. top and sides, top and bottom, bottom and side, etc.).

While detailed discussion of the “open top” embodiment is provided herein, those of ordinary skill in the art will appreciate that many aspects of the “open top” embodiment apply similarly to open bottom embodiments, as well as open side embodiments or embodiments with openings in any other regions or directions, or combinations thereof. Similarly, the device need not remain “open” throughout its use; rather, as several embodiments described herein illustrate, the device may further comprise a cover or seal, which may be affixed reversibly or irreversibly. For example, removal of a removable cover creates an opening, while placement of the cover back on the device closes the device. The opening, and in particular the opening at the top, provides a number of advantages, for example, allowing (i) the creation of one or more gel layers for simulating the application of topical treatments on the cells, tissues, or organs, or (ii) the addition of chemical or biological components such as the seeding of additional cell types for simulated tissue and organ systems. The present disclosure further relates to improvement in fluidic system(s) that improve the delivery of aerosols to simulated tissue and organ systems, such as simulated gastrointestinal tissues.

The present invention contemplates a variety of uses for these open top microfluidic devices and methods described herein. In one embodiment, the present invention contemplates a method of topically testing an agent (whether a drug, food, gas, or other substance) comprising 1) providing a) an agent and b) microfluidic device comprising i) a chamber, said chamber comprising a lumen and projections into the lumen, said lumen comprising ii) a gel matrix anchored by said projections and comprising cell in, on or under said gel matrix, said gel matrix positioned above iii) a porous membrane and under iv) a removable cover, said membrane in contact with v) fluidic channels; 2) removing said removable cover; and 3) topically contacting said cells in, on or under said gel matrix with said agent. In one embodiment, said agent is in an aerosol. In one embodiment, agent is in a liquid, gas, gel, semi-solid, solid, or particulate form. These uses may apply to the open top microfluidic chips described below and herein.

A. Open Top Microfluidic Chips Without Gels.

In one embodiment, open top gut-on-chips do not contain gels, either as a bulk gel or a gel layer. Thus, the present invention also contemplates, in one embodiment, a layered structure comprising i) fluidic channels covered by ii) a porous membrane, said membrane comprising iii) a layer of cells and said membrane positioned below said cells. In one embodiment, there is a removable cover over the cells.

Additional embodiments are described herein that may be incorporated into open top chips without gels.

B. Open Top Microfluidic Chips with Gels.

Furthermore, the present disclosure contemplates improvements to fluidic systems that include a fluidic device, such as a microfluidic device with an open-top region that reduces the impact of stress that can cause the delamination of tissue or related component(s) (e.g., such as a gel layer). Thus, in a preferred embodiment, the open-top microfluidic device comprises a gel matrix. In one embodiment, the open-top microfluidic device does not contain a bulk gel.

The present invention also contemplates, in one embodiment, a layered structure comprising i) fluidic channels covered by ii) a porous membrane, said membrane comprising iii) a layer of cells and said membrane positioned below iv) a gel matrix. In one embodiment, there is a removable cover over the gel matrix (and/or cells). It is not intended that the present invention be limited to embodiments with only one gel or gel layer. In one embodiment, the layered structure further comprises a second gel matrix (e.g. positioned under said membrane). The gel(s) or coatings can be patterned or not patterned. Moreover, when patterned, the pattern need not extend to the entire surface. For example, in one embodiment, at least a portion of said gel matrix is patterned. It is not intended that the present invention be limited by the nature or components of the gel matrix or gel coating. In one embodiment, gel matrix comprises collagen. A variety of thickness is contemplated. In one embodiment of the layered structure, said gel matrix is between 0.2 and 6 mm in thickness.

Also described is a simulated lumen further comprising gel projections into the simulated lumen. Thus, in yet another embodiment, the present invention contemplates a microfluidic device comprising i) a chamber, said chamber comprising a lumen and projections in the lumen, said lumen comprising ii) a gel matrix anchored by said projections, said gel matrix positioned above iii) a porous membrane, said membrane in contact with iv) fluidic channels. In one embodiment, said membrane comprises cells. The projections serve as anchors for the gel. The projections, in one embodiment, project outward from the sidewalls. The projections, in another embodiment, project upward. The projects, in another embodiment, project downward. The projections can take a number of forms (e.g. a T structure, a Y structure, a structure with straight or curving edges, etc.). In some embodiments, there are two or more projections; in other embodiments, there are four or more projections to anchor the gel matrix. In one embodiment, the membrane is above said fluidic channels.

In other embodiments, open top microfluidic chips comprise partial lumens as described herein for closed top chips. Thus, in some embodiments, open top microfluidic chips comprise lumens formed by viscous fingering described herein for closed top chips.

Lumen gel structures may be used in several types of embodiments for open top microfluidic chips, e.g. epithelial cells or parenchymal cells can be attached to outside of the gel, or within the gel. In some embodiments, LPDCs may be added within the gel, below the gel, or above the gel. In some embodiments, stomal cells are added within the gel. In some embodiments, stomal cells are attached to the side of the gel opposite from the lumen. In some embodiments, endothelial cells are located below the gel on the side opposite the lumen. In some embodiments, endothelial cells may be present within the gel.

Additional embodiments are described herein that may be incorporated into open top chips with gels, with or without gels.

All references cited herein are incorporated by reference in their entirety as though fully set forth. Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described.

### III. Chip Activation.

A. Chip Activation Compounds.

In one embodiment, bifunctional crosslinkers are used to attach one or more extracellular matrix (ECM) proteins. A variety of such crosslinkers are available commercially, including (but not limited to) the following compounds:

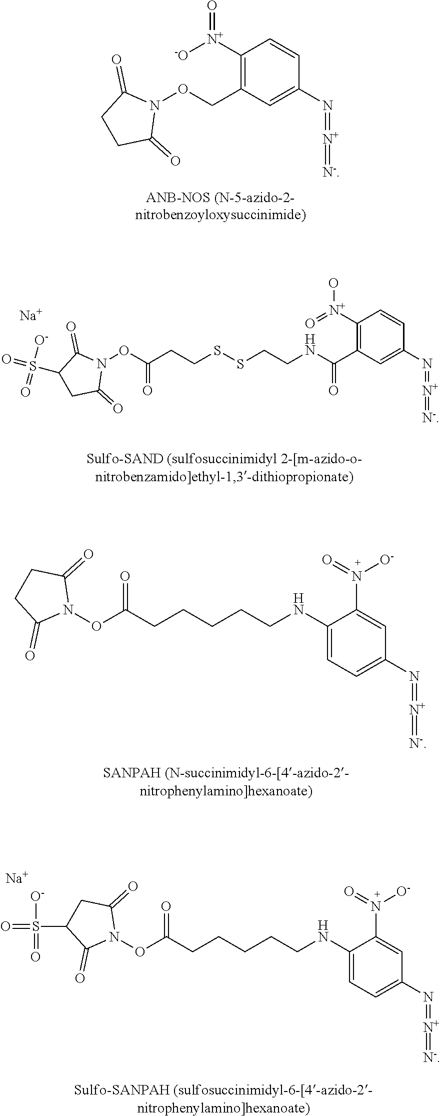

By way of example, sulfosuccinimidyl 6-(4′-azido-2′-nitrophenyl-amino) hexanoate or “Sulfo-SANPAH” (commercially available from Pierce) is a long-arm (18.2 angstrom) crosslinker that contains an amine-reactive N-hydroxysuccinimide (NHS) ester and a photoactivatable nitrophenyl azide. NHS esters react efficiently with primary amino groups (—NH₂) in pH 7-9 buffers to form stable amide bonds. The reaction results in the release of N-hydroxy-succinimide. When exposed to UV light, nitrophenyl azides form a nitrene group that can initiate addition reactions with double bonds, insertion into C—H and N—H sites, or subsequent ring expansion to react with a nucleophile (e.g., primary amines). The latter reaction path dominates when primary amines are present.

Sulfo-SANPAH should be used with non-amine-containing buffers at pH 7-9 such as 20 mM sodium phosphate, 0.15M NaCl; 20 mM HEPES; 100 mM carbonate/bicarbonate; or 50 mM borate. Tris, glycine or sulfhydryl-containing buffers should not be used. Tris and glycine will compete with the intended reaction and thiols can reduce the azido group.

For photolysis, one should use a UV lamp that irradiates at 300-460 nm. High wattage lamps are more effective and require shorter exposure times than low wattage lamps. UV lamps that emit light at 254 nm should be avoided; this wavelength causes proteins to photodestruct. Filters that remove light at wavelengths below 300 nm are ideal. Using a second filter that removes wavelengths above 370 nm could be beneficial but is not essential.

B. Exemplary Methods of Chip Activation.

Prepare and sanitize hood working space.

1. S-1 Chip (alternatively a tall channel chip): Handling—Use aseptic technique, hold Chip using a Carrier.

a. Use 70% ethanol spray and wipe the exterior of Chip package prior to bringing into hood.

b. Open package inside hood.

c. Remove Chip and place in sterile petri dish (6 Chips/Dish).

d. Label Chips and Dish with respective condition and Lot #

2. Surface Activation with Chip Activation Compound (light and time sensitive).
a. Turn off light in biosafety hood
b. Allow vial of Chip Activation Compound powder to fully equilibrate to ambient temperature (to prevent condensation inside the storage container, as reagent is moisture sensitive).
c. Reconstitute the Chip Activation Compound powder with ER-2 (buffer) solution.
   i. Add 10 ml Buffer, such as HEPES, into a 15 ml conical covered with foil.
   ii. Take 1 ml Buffer from above conical and add to chip Activation Compound (5 mg) bottle, pipette up and down to mix thoroughly and transfer to same conical.
   iii. Repeat 3-5 times until chip Activation Compound is fully mixed. NOTE: Chip Activation Compound is single use only, discard immediately after finishing Chip activation, solution cannot be reused.
d. Wash channels:
   i. Inject 200 μl of 70% ethanol into each channel and aspirate to remove all fluid from both channels.
   ii. Inject 200 μl of Cell Culture Grade Water into each channel and aspirate to remove all fluid from both channels.
   iii. Inject 200 μl of Buffer into each channel and aspirate to remove fluid from both channels
e. Inject Chip Activation Compound Solution (in buffer) in both channels.
   i. Use a P200 and pipette 200 ul to inject Chip Activation Compound/Buffer into each channel of each chip (200 ul should fill about 3 Chips (Both Channels)).
   ii. Inspect channels by eye to be sure no bubbles are present. If bubbles are present, flush channel with Chip Activation Compound/Buffer until bubbles have been removed.
f. UV light activation of Chip Activation Compound
   i. Place Chips into UV light box:
   ii. UV light treat Chips for 20 min
g. While the Chips are being treated, prepare ECM Solution.
   i. After UV treatment, gently aspirate Chip Activation Compound/Buffer from channels via same ports until channels are free of solution.
   ii. Carefully wash with 200 ul of Buffer solution through both channels and aspirate to remove all fluid from both channels
   iii. Carefully wash with 200 ul of sterile DPBS through both channels
   iv. Carefully aspirate PBS from channels and move on to: ECM-to-Chip.

References incorporated by reference in their entirety include: Corpening et al., Dev Dyn. 2008 April; 237(4): 1119-1132 and Lee et al., Nat Prot, 2010 March; Vol 5: 688-701.

### Experimental

Intestine-Chip Epithelial Tissue Sources: includes but are not limited to: Caco-2 BBE; Primary Enteroids; iPSC Organoids, see FIGS. 1E-G.
Cell Sources: Primary cells; Organoids; iPSC-Derived; etc.
Intestine-Chips can be generated from patient biopsies enabling the study of disease mechanisms and drug efficacy testing. Including but not limited to personalized medicine.
Incorporating Lamina Propria Derived Immune Cells in some embodiments of Intestine-On-Chip. Incorporated Lamina Propria Immune Cells (CD45+) from at least Two Donors: Healthy Individual (hLP); Patient with Ulcerative Colitis (UC LP).
Incorporating Lamina Propria Derived Immune Cells in some embodiments of Intestine-On-Chip. Immune cells isolated from UC donor taken from non-inflamed and inflamed regions: Non-Inflamed (niLP) Inflamed (iLP).
Human Primary Resident Immune Cells Retain In Vivo Phenotype. Ulcerative colitis (UC) patient immune cells derived from regions of non-inflamed (niLP) and inflamed lamina propria (iLP) retain their inflammatory phenotype in the Intestine-Chip; Increased pro-inflammatory cytokines secretion and weaker barrier function.

### EXAMPLE 1

Adhesion Assay and Bacterial Counting Protocol (Method).

Exemplary on-chip infection and analysis of surface-adherent bacteria. Bacteria from log phase cultures are collected and washed with PBS. Three (3)×10⁶ CFU/chip per strain are added to airway chips at an MOI of approximately 10. Where the multiplicity of infection or MOI refers to the ratio of microbial agents (e.g. virus, bacteria) to infection targets, in this case chips containing airway cells. Infect for 1 hour (1 hpi), wash 3× in PBS to remove nonadherent bacteria. Cells are trypsinized gently so as to avoid lysing them, therefore intracellular bacteria do not contribute to the CFU counts. Cell samples are collected, e.g. washed, from chips then vortexed to disassociate cell clumps. Samples are serially diluted for CFU quantification. At least N=3 per treatment.

In order to quantify both extracellular and intracellular bacteria concurrently from a single chip, after infection unattached cells in the inoculum is removed, wash 3× with PBS and lyse cells (but not bacteria) with 1% triton. This allows counting surface-associated and intracellular bacteria cell numbers.

In order to quantify mainly intracellular bacteria, cells are treated with antibiotics to kill extracellular bacteria, antibiotics are removed, cells are washed and lysed with 1% triton. The CFU counts obtained in this assay represent intracellular bacteria counts as extracellular bacteria are killed.

FIGS. 6A-B shows exemplary real time imaging after infection of one embodiment of an Airway Chip with bacteria P. aeruginosa infection on chip. Both pseudomonas strains, wild-type (WT) and mutant, form micro-colonies/aggregates on airway chip. Bacterial inoculum is plated and CFU are counted to ensure target MOI. Images are acquired 24 hpi. FIG. 6A PA 5919-WT. FIG. 6B PA 5890-Mutant. Left: Alexa fluor 488—anti-P. aeruginosa (Pa) antibody. Central: Bright-field. Right: Alexa fluor 488—anti-P. aeruginosa (Pa) antibody-Bright-field.

### EXAMPLE 2

Exemplary On-Chip Infection Protocol.

Bacteria from log phase cultures are collected and washed with PBS.

Three (3)×10⁶ CFU/chip per strain are infected at an MOI of approximately 10. Infect for 2 h, wash 3× PBS to remove nonadherent bacteria.

Mature small airway chips at air-liquid interface (ALI) are gently washed with PBS to remove excess mucus. Cells are inoculated with 2-3×10⁶ colony forming units (CFUs) in Hank's balanced salt solution (HBSS) for 2 hours. At the end of the incubation, the inoculum is removed and the cells are washed 3× with phosphate buffer saline (PBS) to remove any nonadherent bacteria. PBS is removed and cells are incubated at ALI up to 24 hours (h) under flow in the bottom channel but not in the top channel.

FIGS. 7A-C shows exemplary immunofluorescence, with Z-stacks or a side view, after infection of one embodiment of an Airway Chip with bacteria P. aeruginosa infection on chip. Pseudomonas establishes an intracellular niche as well as forming extracellular micro-colonies on the epithelial cell surface. Z-stacks are shown as a bar across the top (to the right of the 24 h label, and the down the right side of the micrographs. The upper (or right side on the side bar) part of the Z-stacks represent apical regions then down through the cells to the basil regions at the bottom of bar (or left side of the side bar). These Z-stacks indicate that bacterium are intracellularly located. FIG. 7A PA 5919-WT. Actin (red); Pa (green); DAPI (blue). FIG. 7B PA 5890-Mutant. Actin (pink); Pa (green); DAPI (blue). Images are acquired at 24 hpi. FIG. 7C shows a confocal immunofluorescent micrograph side view of a cell layer infected with P. aeruginosa in a microfluidic airway chip, 24 hours post infection. Actin (pink); Pa (green); DAPI (blue). Bacterial aggregates on apical surface as well as intracellular bacteria are observed.

### EXAMPLE 3

Microfluidic Chip Emulates Damage Induced by Infectious Microbes: i.e. Changes in Mucociliary Activity.

Observing Changes in Mucociliary Activity, i.e. Readouts.

FIGS. 8A-C shows exemplary mucociliary activity photographed in bright field on one embodiment of a Pseudomonas infection on chip. Micrographs represent one image from a video of cilia beating on-chip. FIG. 8A Non-infected control microfluidic chip image representing beating cilia. FIG. 8B PA 5890-Mutant infected microfluidic chip image representing loss of beating cilia. FIG. 8C PA 5919-WT microfluidic chip image also representing a loss of beating cilia.

FIGS. 9 shows an exemplary comparison of cilia beating frequency (CBF) between Pseudomonas strains in one embodiment of a Pseudomonas infection on chip. Images from a video of epidermal cells' cilia beating on-chip are quantitatively evaluated showing that both wild type and mutant strains has altered cilia beating frequency compared to controls without added bacteria.

FIGS. 10 an exemplary comparison of cellular cilia coverage after infection with Pseudomonas strains in one embodiment of a Pseudomonas infection on chip. Mutant (increases) and WT (decreases) show significant differences in density compared to controls.

FIG. 11 shows an exemplary Bacterial adherence on chip in one embodiment of a microfluidic airway epithelia. P. aeruginosa WT (MB5980) and mutant (MB5919) strains adhere to airway epithelium at similar rates. Unpaired t-tests p=0.0641. N=3.

### EXAMPLE 4

Treatment of Microfluidic Chip with an Anti-Microbial Compound.

After an exemplary on-chip infection protocol, including removing nonadherent bacteria, a test compound is immediately added to the top (apical) and bottom (basal) channels then cells are cultured under flow using media containing the anti-microbial compound, for 24 hours. Exemplary test compound amounts are at least 0.1 μg/ml, up to 50 μg/ml, up to 100 μg/ml, up to 500 μg/ml. In one embodiment, an exemplary test compound is a drug Imipenem.

More specifically, in one embodiment of an Airway on-chip, duplicate chips are infected with PA 5919 WT or PA 5890 Mutant as described herein. Then, Imipenem is added to apical and basolateral fluids (cell media) for treatment with Imipenem at 50, 100 and 500 μg/ml for 24 hours with at least one chip without Imipenem. At 24 h post treatment, wash cells 3× to remove the test drug, i.e. antibiotic Imipenem. Cells lysed with 1% Triton for 10 minutes on chip, then the lyste is collected from the chip's channels. Lysates are serially diluted for CFU quantification. N=2

Assay readouts: Viability of extracellular bacteria (apical sampling). Viability of intracellular bacteria (cell lysis and sampling). Two-way ANOVA with Dunnett's post-test **<0.05, **<0.001 (compared to untreated).

Imipenem treatment has significant bactericidal effect on MB5890 (mutant) strain and MB5919 (WT) growth. Imipenem kills most of the extracellular bacteria (this is determined by plating out apical supernatants), however does not rule out that there are still surface-associated extracellular bacteria on the epithelium. There is reduction in total bacterial counts (intra- and/or extracellular bacteria) but at this stage we cannot determine if bacteria remaining post Imipenem treatment are both intra and extracellular. However, the results clearly show P. aeruginosa killing post antibiotic treatment as indicated in CFU counts and that the bacteria can persist in small airway cells over time.

Airway cells are impermeable to Imipenem at lower concentrations. P. aeruginosa can persist in small airway cells over time when treated with 50 μg/ml Imipenem.

FIGS. 12A-B shows an exemplary Imipenem (Merck compound) effects on P. aeruginosa infection. FIG. 12A shows exemplary Imipenem (Merck compound) effects on P. aeruginosa infection in a Transwell culture. FIG. 12B Imipenem treatment reduces total bacterial counts via bacterial killing in one embodiment of a P. aeruginosa infection on chip. Two-way ANOVA with Dunnett's post-test **<0.05, **<0.001 (compared to untreated).

FIGS. 13A-C shows exemplary Imipenem (Merck compound) effects on P. aeruginosa infection, WT vs. mutant, on airway cells in Transwells. FIG. 13A shows exemplary Imipenem treatment. FIG. 13B shows exemplary Carbenicillin treatment. FIG. 513C shows exemplary Tetracycline treatment. Two-way ANOVA with Dunnett's post-test **<0.05, **<0.001, ***<0.0001 (compared to untreated).

FIGS. 14A-C shows exemplary Real time imaging of Imipenem effects on P. aeruginosa infection on one embodiment of a PA 5919 WT Pseudomonas infection on chip. FIG. 14A untreated (noninfected) control. FIG. 14B 50 ug/ml. FIG. 14C 500 ug/m. PA 5919 WT 24 hpi. Left: Alexa fluor 488—anti-P. aeruginosa (Pa) antibody. Central: Bright-field. Right: Alexa fluor 488—anti-P. aeruginosa (Pa) antibody-Bright-field.

FIGS. 15A-C shows exemplary Real time imaging of Imipenem effects on P. aeruginosa infection on one embodiment of a PA 5890 Mutant 24 hpi Pseudomonas infection on chip. FIG. 15A untreated (noninfected) control. FIG. 15B 50 μg/ml. FIG. 15C 500 μg/m. PA 5890 Mutant 24 hpi. Left: Alexa fluor 488—anti-P. aeruginosa (Pa) antibody. Central: Bright-field. Right: Alexa fluor 488—anti-P. aeruginosa (Pa) antibody-Bright-field.

### EXAMPLE 5

### Measuring Human β-Defensin 2 Secretion Post-Infection.

Prolonged P. aeruginosa Infection Results in Enhancement of HBD-2 Protein Secretion.

Human beta-defensin 2 is measured in epithelial apical secretions using ELISA. Significance is **<0.05 according to an unpaired t-test.

FIG. 16 shows an exemplary secretion of Human β-Defensin 2 post P. aeruginosa infection on one embodiment of a microfluidic Airway Chip. For comparison, HBD-2 protein (pg/ml) are measured in control chips; after WT P. aeruginosa; and a mutant strain of P. aeruginosa are tested for Human β-Defensin 2 secretion in apical wash, 24 hpi. Unpaired t-test, **<0.05. N=2.

### EXAMPLE 6

### Induction of Apoptosis in Host Cells Post P. Aeruginosa Infection.

One embodiment of an Airway On-Chip is infected as described above for 2 hours and then treated 50 μg/ml tetracycline for 24 h (top and bottom channels under flow) for 24 h in order to limit bacterial replication (to avoid over-replication and cell lifting). Using this methodology, we performed longer infections on an airway chip and avoided cell death at 24 h. At 24 h, chips are fixed with 4% paraformaldehyde for 15 minutes and subsequently permeabilized with 0.25% triton in PBS. Apoptosis staining is performed following the Click-iT™ Plus TUNEL Assay for In Situ Apoptosis Detection, Alexa Fluor 647 assay: Click-iT™ Plus TUNEL Assay, Thermo Fisher Scientific Inc., 6 November 2017.

The TUNEL assay is based on the incorporation of modified dUTPs by the enzyme terminal deoxynucleotidyl transferase (TdT) at the 3′-OH ends of fragmented DNA, a hallmark as well as the ultimate determinate of apoptosis. Click-iT™ Plus TUNEL assay with the Alexa Fluor™ 647 dye is utilized to detect the fragment DNA (purple), nuclei are labelled with DAPI. Uninfected cells show very limited apoptosis, whereas P. aeruginosa infected chips exhibited a significant increase in the number of apoptotic cells.

Staurosporin, a prototypical ATP-competitive kinase inhibitor, is used as a positive control in this assay. Chips incubated with 3 μM staurosporin for 3 h at 37° C. had increased apoptotic cell numbers. As an additional positive control, chips are treated with 1 unit of DNAse I for 30 minutes at room temperature to induce TUNEL positive DNA strand breaks. As shown in the figure, almost all DNAse I treated cells underwent apoptosis post treatment.

FIGS. 17A-D shows exemplary apoptosis via TUNEL staining at 24 h post infection. Apoptotic, TUNEL+, (pink); nuclei, DAPI+, (blue). FIG. 17A uninfected; FIG. 17B Pa infected; FIG. 17C staurosporin treatment. Staurosporin refers to an ATP-competitive kinase inhibitor. FIG. 17D DNAse I treatment. DNAse I refers to an endonuclease that nonspecifically cleaves DNA to release di-, tri- and oligonucleotide products with 5′-phosphorylated and 3′-hydroxylated ends. DNase I acts on single- and double-stranded DNA, chromatin and RNA:DNA hybrids.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in medicine, molecular biology, cell biology, genetics, statistics or related fields are intended to be within the scope of the following claims.

1-40. (canceled)

41. A method of culture, comprising:
a) providing colonic epithelium and tissue specific endothelial cells on opposing sides of a membrane; and
b) alternating exposure of the colonic epithelium to culture medium and air.

42. The method of claim 41, wherein exposing said colonic epithelium to culture medium comprising immersing cells in culture medium.

43. The method of claim 41, wherein exposing said colonic epithelium to air comprises exposing cells to an air-liquid interface.

44. The method of claim 41, wherein said alternating exposure results in a greater number of cells differentiating into Goblet cells as compared to colonic epithelium exposed constantly to culture media and without exposure to air.

45. A method of culturing, comprising: a) providing i) a microfluidic device comprising a first microchannel and a second microchannel separated by a membrane, said membrane comprising first and second sides, wherein said first side serves as a surface for said first microchannel and said second side serves as a surface for said second microchannel and ii) first living cells attached to said first side of said membrane; b) introducing a fluid at a flow rate into said first microchannel so as to create a flow period; c) stopping said fluid flow so as to create a static period; d) introducing air into said first microchannel during said static period so as to provide an air-liquid interface for said living cells; and e) alternating culture between said flow period and said static period.

46. The method of claim 45, wherein said living cells are intestinal cells.

47. The method of claim 46, wherein said intestinal cells are colonic epithelial cells that differentiate during said culture.

48. The method of claim 47, wherein said alternating of step e) results in a greater number of cells differentiating into Goblet cells as compared to colonic epithelial cells exposed constantly to culture media and without exposure to air.

49. The method of claim 45, wherein said introducing a fluid of step b) results in the immersing of said living cells in said fluid.

50. The method of claim 45, further comprising after step b), causing cyclic stretching of said membrane at a frequency level.

51. The method of claim 50, wherein said cyclic stretching is stopped at step c).

52. The method of claim 45, wherein said static period extends for a twenty-four hour time period.

53. The method of claim 52, wherein said air-liquid interface of said static period extends for the first six hours of said 24 hour time period.

54. The method of claim 45, wherein said alternating of step e) is done up to three times.

55. The method of claim 45, wherein said alternating of step e) is done up to four times.

56. The method of claim 45, further comprising inoculated said microfluidic device with one or more microorganisms capable of developing biofilms.

57. The method of claim 56, wherein said microorganisms are bacterial cells.

58. The method of claim 57, wherein said bacterial cells adhere to said first living cells.

59. The method of claim 58, wherein said bacterial cells are selected from the group consisting of nonpathogenic bacterial strains and pathogenic bacterial strains.

60. A method of culturing, comprising:
a) providing i) a microfluidic device comprising a first microchannel and a second microchannel separated by a membrane, said membrane comprising first and second sides, wherein said first side serves as a surface for said first microchannel and said second side serves as a surface for said second microchannel and ii) first living cells attached to said first side of said membrane;
b) introducing a fluid at a flow rate into said first microchannel so as to create a flow period;
c) causing cyclic stretching of said membrane at a frequency level;
d) stopping said fluid flow and said cyclic stretching so as to create a static period wherein air is introduced into said first microchannel so as to provide an air-liquid interface; and e) alternating culture between said static period and said flow period with combined steps b) and c).

61. The method of claim 60, wherein said living cells are intestinal cells.

62. The method of claim 61, wherein said intestinal cells are colonic epithelial cells that differentiate during said culture.

63. The method of claim 62, wherein said alternating of step e) results in a greater number of cells differentiating into Goblet cells as compared to colonic epithelial cells exposed constantly to culture media and without exposure to air.

64. The method of claim 60, wherein said introducing a fluid of step b) results in the immersing of said living cells in said fluid.

65. The method of claim 60, wherein said cyclic stretching comprises mechanical stretching at a frequency of 0.15 Hz.

66. The method of claim 60, wherein said static period extends for a twenty-four hour time period.

67. The method of claim 66, wherein said air-liquid interface of said static period extends for the first six hours of said 24 hour time period.

68. The method of claim 60, wherein said alternating of step e) is done up to three times.

69. The method of claim 60, wherein said alternating of step e) is done up to four times.

70. The method of claim 60, further comprising inoculating said microfluidic device with one or more microorganisms capable of developing biofilms.

71. The method of claim 70, wherein said microorganisms are bacterial cells.

72. The method of claim 71, wherein said bacterial cells adhere to said first living cells.

73. The method of claim 72, wherein said bacterial cells are selected from the group consisting of nonpathogenic bacterial strains and pathogenic bacterial strains.

74. A method of identifying a pathogenic bacteria strain, comprising:
a) providing:
i) a microfluidic device;
ii) a plurality of living mammalian cells; and
iii) a plurality of bacterial cells from a pathogenic bacteria strain;
b) seeding said living mammalian cells in said microfluidic device so as to provide a cell layer;
c) contacting said cell layer with said plurality of bacterial cells so as to allow at least some of said bacterial cells to adhere to said cell layer, so as to create contacted cells; and
d) detecting a change in said contacted cells, thereby identifying said bacterial cells as pathogenic.

75. The method of claim 74, wherein said living mammalian cells comprise intestinal cells.

76. The method of claim 74, wherein said living mammalian cells comprise airway epithelial cells.

77. The method of claim 76, wherein said airway epithelial cells display cilia beating at a frequency after step b).

78. The method of claim 77, wherein said detecting a change comprises detecting a change in cilia beating frequency.

79. The method of claim 74, wherein said pathogenic bacterial strain is a Pseudomonas strain.

80. The method of claim 74, wherein said microfluidic device comprises first and second microchannels separated by a membrane, said membrane comprising first and second sides, wherein said first side serves as a surface for said first microchannel and said second side serves as a surface for said second microchannel.

81. The method of claim 74, wherein the method further comprises introducing fluid at a flow rate prior to, during or after step c).

82. The method of claim 75, wherein said change detected in step d) comprises a decrease in said barrier function of said contacted intestinal cells.

83. The method of claim 82, wherein said decrease in barrier function is measured by a decrease in the expression of one or more tight junction proteins.

84. The method of claim 75, wherein said intestinal cells are primary epithelial cells.

85. The method of claim 75, wherein said intestinal cells are patient derived cells.
